(19) United States
(12) Patent Application Publication
(10) Pub. No.: US 2013/0090349 A1
(43) Pub. Date: Apr. 11, 2013

(54) Tamper-resistant oral pharmaceutical dosage form comprising opioid agonist and opioid antagonist

(71) Applicant: Grünenthal GmbH, Aachen (DE)

(72) Inventors: Anja GEIßLER, Stolberg (DE); Lutz BARNSCHEID, Monchengladbach (DE)

(73) Assignee: Grünenthal GmbH, Aachen (DE)

(21) Appl. No.: 13/645,595

(22) Filed: Oct. 5, 2012

Related U.S. Application Data
(60) Provisional application No. 61/543,869, filed on Oct. 6, 2011.

(30) Foreign Application Priority Data
Oct. 6, 2011 (EP) 11 008 131.2
Nov. 16, 2011 (EP) 11 009 090.9
Feb. 28, 2012 (EP) 12 001 297.6

(51) Int. Cl. A61K 47/10

(52) U.S. Cl. CPC A61K 47/10
USPC 514/282

(57) ABSTRACT

A pharmaceutical dosage form for oral administration having a breaking strength of at least 300 N and comprising an opioid agonist, an opioid antagonist, and a polyalkylene oxide having an average molecular weight of at least 200,000 g/mol, wherein in accordance with Ph. Eur. the in vitro release profile of the opioid agonist essentially corresponds to the in vitro release profile of the opioid antagonist, and wherein the opioid agonist and the opioid antagonist are intimately mixed with one another and homogeneously dispersed in the polyalkylene oxide. The pharmaceutical dosage form is useful, for example, to treat pain in a patient in need of such treatment.

This application claims priority of U.S. Provisional Application Ser. No. 61/543,869, filed on Oct. 6, 2011, European Patent Application No. 11 008 131.2, filed on Oct. 6, 2011, European Patent Application No. 11 009 090.9, filed on Nov. 16, 2011, and European Patent Application No. 12 001 297.6, filed on Feb. 28, 2012, the entire contents of which patent applications are incorporated herein by reference.

The invention relates to a pharmaceutical dosage form for oral administration having a breaking strength of at least 300 N and comprising an opioid agonist, an opioid antagonist, and a polyalkylene oxide having an average molecular weight of at least 200,000 g/mol, wherein in accordance with Ph. Eur. the in vitro release profile of the opioid agonist essentially corresponds to the in vitro release profile of the opioid antagonist, and wherein the opioid agonist and the opioid antagonist are intimately mixed with one another and homogeneously dispersed in the polyalkylene oxide.

Tamper-resistant pharmaceutical dosage forms containing opioid agonists have been known for many years. Some concepts of rendering pharmaceutical dosage forms tamper resistant rely on the presence of opioid antagonists.

In some embodiments, the opioid agonist is provided in releasable form and the opioid antagonist is sequestered and not released when the pharmaceutical dosage form is administered in the prescribed manner, i.e. intact and orally. Only when the pharmaceutical dosage form is tampered with, e.g. by mechanical disruption such as pulverization, the opioid antagonist is released from the pharmaceutical dosage form thereby evolving its antagonizing effect and avoiding misuse of the opioid agonist.

In other embodiments, the opioid antagonist is released from the pharmaceutical dosage form upon prescribed administration, e.g. oral administration, but due to its chemical nature, pharmacokinetic properties, and pharmacodynamic properties, the antagonizing effect of the opioid antagonist does not evolve. This can be achieved by employing opioid antagonists that have no or only a very poor bioavailability when being administered by the prescribed route, e.g. orally. Only when the pharmaceutical dosage form is tampered with, e.g. by liquid extraction of the constituents and administration of the liquid extract by another route, typically parenterally such as intravenously, the opioid antagonist has a sufficient bioavailability so that it evolves its antagonizing effects and can avoid misuse of the opioid agonist.

Other concepts of rendering pharmaceutical dosage forms tamper resistant rely on the mechanical properties of the pharmaceutical dosage forms, particularly a substantially increased breaking strength (resistance to crushing). The major advantage of such pharmaceutical dosage forms is that comminuting, particularly pulverization, by conventional means, such as grinding in a mortar or fracturing by means of a hammer, is impossible or at least substantially impeded. Thus, by conventional means that are available to an abuser, such pharmaceutical dosage forms cannot be converted into a form suitable for abuse, e.g. a powder for nasal administration.

Such pharmaceutical dosage forms may additionally contain aversive agents such as opioid antagonists, which are locally separated from the opioid agonist in the pharmaceutical dosage form, i.e. the pharmaceutical dosage forms comprise subunits containing opioid agonist but no opioid antagonist, and other subunits containing opioid antagonist but no opioid agonist. When these pharmaceutical dosage forms are administered in a prescribed manner, the opioid antagonist is not released from the pharmaceutical dosage form and thus, does not exhibit any effect. In this regard it can be referred to e.g., WO 2005/016313, WO 2005/016314, WO 2005/063214, WO 2005/102286, WO 2006/002883, WO 2006/002884, WO 2006/002886, WO 2006/082097, WO 2006/082099, and WO 2008/107149.

These known tamper resistant pharmaceutical dosage forms are not satisfactory in every respect. Manufacture is complicated and laborious, as different subunits need to be prepared separately and are mixed with one another subsequently, before the final pharmaceutical dosage form is formed. Under these circumstances, content uniformity and other requirements are difficult to satisfy. Furthermore, the release profile of the opioid agonist typically differs from that of the opioid antagonist. This is because due to their different chemical nature, the dispersibility of the opioid agonist in the other excipients of the pharmaceutical dosage form typically differs from the dispersibility of the opioid antagonist. The same applies to their solubility in the release medium.

WO 2010/140007 A2 discloses tamper-resistant dosage forms comprising a matrix and melt-extruded particulates comprising a drug that are present as a discontinuous phase in said matrix.

US 2005/0245556 A1 relates to storage stable pharmaceutical preparations comprising oxycodone and naloxone for use in pain therapy from which the active compounds are released in a sustained, invariant and independent manner.

Dosage forms comprising oxycodone hydrochloride and naloxone hydrochloride and providing sustained release of at least the oxycodone hydrochloride are known from US 2003/0069263 A1.

There is a demand for tamper resistant pharmaceutical dosage forms containing opioid agonists and having advantages compared to the pharmaceutical dosage forms of the prior art.

This object has been achieved by the subject-matter described hereinbelow.

A first aspect of the invention relates to a pharmaceutical dosage form for oral administration having a breaking strength of at least 300 N and comprising an opioid agonist, an opioid antagonist, and a polyalkylene oxide having an average molecular weight of at least 200,000 g/mol, wherein, when the pharmaceutical dosage form is not tampered with, in accordance with Ph. Eur. the in vitro release profile of the opioid agonist essentially corresponds to the in vitro release profile of the opioid antagonist, and wherein the opioid agonist and the opioid antagonist are intimately mixed with one another and homogeneously dispersed in the polyalkylene oxide.

It has been surprisingly found that the following objects concerning tamper resistance can be achieved simultaneously by means of the pharmaceutical dosage form according to the invention:
(i) when the pharmaceutical dosage form is not tampered with and is administered by the prescribed oral route, the opioid agonist develops its desired pharmacological effect and the opioid antagonist, which is simultaneously released, does not counter this effect of the opioid agonist, especially as the opioid antagonist is preferably very poorly or not bioavailable when being administered orally. Nevertheless, in the intestine the orally administered opioid antagonist can locally block the opioid receptors thereby preventing obstipation, an undesired adverse event otherwise occurring due to induction by the opioid agonist;
(ii) when the pharmaceutical dosage form is tampered with by liquid extraction of the active ingredients and is then administered by the non-prescribed, parenteral route, the opioid antagonist is fully bioavailable and thus, fully develops its antagonizing effect thereby avoiding misuse of the opioid agonist;
(iii) when attempts are made to mechanically disrupt the pharmaceutical dosage form by conventional means typically available to an abuser, particularly in order to prepare a powder that is suitable for e.g. nasal administration, such attempts fail due to the increased breaking strength of the pharmaceutical dosage form.

### BRIEF DESCRIPTION OF THE DRAWINGS

The invention will now be described in greater detail with reference to the drawings, wherein:

FIG. 1 is the in vitro release profile of the pharmaceutical dosage form according to Example 1-1 (▪ drug release Oxycodone; ♦ drug release Naloxone);

FIG. 2 is the in vitro release profile of the pharmaceutical dosage form according to Example 1-2 (▪ drug release Oxycodone; ♦ drug release Naloxone);

FIG. 3 is the in vitro release profile of the pharmaceutical dosage form according to Example 1-3 (▪ drug release Oxycodone; ♦ drug release Naloxone);

FIG. 4 is the in vitro release profile of the pharmaceutical dosage form according to Example 1-4 (▪ drug release Oxycodone; ♦ drug release Naloxone);

FIG. 5 is the in vitro release profile of the pharmaceutical dosage form according to Example 2-1 (▪ drug release Oxycodone; ♦ drug release Naloxone);

FIG. 6 is the in vitro release profile of the pharmaceutical dosage form according to Example 2-2 (▪ drug release Oxycodone; ♦ drug release Naloxone);

FIG. 7 is the in vitro release profile of the pharmaceutical dosage form according to Example 2-3 (▪ drug release Oxycodone; ♦ drug release Naloxone);

FIG. 8 is the in vitro release profile of the pharmaceutical dosage form according to Example 2-4 (▪ drug release Oxycodone; ♦ drug release Naloxone);

FIG. 9 is the in vitro release profile of the pharmaceutical dosage form according to Example 2-5 (▪ drug release Oxycodone; ♦ drug release Naloxone);

FIG. 10 is the in vitro release profile of the pharmaceutical dosage form according to Example 2-6 (▪ drug release Oxycodone; ♦ drug release Naloxone);

FIG. 11 is the in vitro release profile of the pharmaceutical dosage form according to Example 3-1 (▪ drug release Oxycodone; ♦ drug release Naloxone);

FIG. 12 is the in vitro release profile of the pharmaceutical dosage form according to Example 3-2 (▪ drug release Oxycodone; ♦ drug release Naloxone);

FIG. 13 is the in vitro release profile of the pharmaceutical dosage form according to Example 3-3 (▪ drug release Oxycodone; ♦ drug release Naloxone);

FIG. 14 is the in vitro release profile of the pharmaceutical dosage form according to Example 4-1 (▪ drug release Oxycodone; ♦ drug release Naloxone);

FIG. 15 is the in vitro release profile of the pharmaceutical dosage form according to Example 4-2 (▪ drug release Oxycodone; ♦ drug release Naloxone);

FIG. 16 is the in vitro release profile of the pharmaceutical dosage form according to Example 4-3 (▪ drug release Oxycodone; ♦ drug release Naloxone);

FIGS. 17A and 17B show the in vitro release profiles of commercially available Targin tablets;

FIGS. 18A and 18B show corresponding in vitro release profiles using inventive formulation 3-3;

FIGS. 19A and 19B show corresponding in vitro release profiles using inventive formulation 4-3;

FIG. 20 is the in vitro release profile of the pharmaceutical dosage form according to Example 6-1 (▪ drug release Oxycodone; ♦ drug release Naloxone);

FIG. 21 is the in vitro release profile of the pharmaceutical dosage form according to Example 6-2 (▪ drug release Oxycodone; ♦ drug release Naloxone);

FIG. 22 is the in vitro release profile of the pharmaceutical dosage form according to Example 6-3 (▪ drug release Oxycodone; ♦ drug release Naloxone);

FIG. 23 is the in vitro release profile of the pharmaceutical dosage form according to Example 6-4 (▪ drug release Oxycodone; ♦ drug release Naloxone);

FIG. 24 is the in vitro release profile of the pharmaceutical dosage form according to Example 6-5 (▪ drug release Oxycodone; ♦ drug release Naloxone);

FIG. 25 is the in vitro release profile of the pharmaceutical dosage form according to Example 6-6 (▪ drug release Oxycodone; ♦ drug release Naloxone);

FIG. 26 is the in vitro release profile of the drug release of hydromorphone HCl and naloxone HCl of intact tablets in simulated gastric fluids;

FIG. 27 is the in vitro release profile of the drug release of Hydromorphone and Naloxone of intact tablets in 40% ethanol;

FIG. 28 is the in vitro release profile of the drug release of Hydromorphone and Naloxone of manipulated tablets in simulated gastric fluids;

FIG. 29 is the in vitro release profile of the drug release of Hydromorphone and Naloxone of manipulated tablets in 40% ethanol;

FIG. 30 is the in vitro release profile of the drug release of Oxycodone and Naloxone of intact tablets in simulated gastric fluids (HCl);

FIG. 31 is the in vitro release profile of the drug release of Oxycodone and Naloxone of intact tablets in 40% ethanol;

FIG. 32 is the in vitro release profile of the drug release of Oxycodone and Naloxone of manipulated tablets in simulated gastric fluids; and

FIG. 33 is the in vitro release profile of the drug release of Oxycodone and Naloxone of manipulated tablets in 40% ethanol.

Preferably, the opioid agonist and the opioid antagonist are homogeneously distributed over the pharmaceutical dosage form or, when the pharmaceutical dosage form comprises a film coating, over the coated core of the pharmaceutical dosage form.

The opioid agonist and the opioid antagonist are intimately mixed with one another and homogeneously dispersed in the polyalkylene oxide, preferably in molecular disperse form.

Preferably, the opioid agonist is not locally separated from the opioid antagonist. Preferably, the pharmaceutical dosage form contains neither any subunits comprising opioid agonist but no opioid antagonist, nor any subunits comprising opioid antagonist but no opioid agonist.

Preferably, the opioid agonist and the opioid antagonist are embedded in a prolonged release matrix comprising the polyalkylene oxide. Thus, the prolonged release matrix is preferably a hydrophilic matrix. It has been surprisingly found that the release of the opioid agonist and the opioid antagonist from the prolonged release matrix relies on a combined mechanism that is regulated by erosion and diffusion of the release medium into the matrix.

Preferably, the release profile of the opioid agonist is matrix-retarded. Preferably, the opioid agonist is embedded in a matrix comprising the polyalkylene oxide, said matrix controlling the release of the opioid agonist from the pharmaceutical dosage form.

Physiologically acceptable materials which are known to the person skilled in the art may be used as supplementary matrix materials. Polymers, particularly preferably cellulose ethers and/or cellulose esters are preferably used as hydrophilic matrix materials. Ethylcellulose, hydroxypropylmethylcellulose, hydroxypropylcellulose, hydroxymethylcellulose, hydroxyethyl-cellulose, and/or the derivatives thereof, such as the salts thereof are very particularly preferably used as matrix materials.

Preferably, the prolonged release matrix does not contain substantial amounts of (i.e. more than 5 wt.-%, relative to the total weight of the prolonged release matrix), more preferably does not contain any (meth)acrylic polymers, e.g. neutral copolymers of ethyl acrylate and methyl methacrylate such as Eudragit® NE 40 D.

Preferably, the relative weight ratio of the polyalkylene oxide to the opioid agonist is at least 0.5:1, more preferably at least 1:1, at least 2:1, at least 3:1, at least 4:1, at least 5:1, at least 6:1, at least 7:1, at least 8:1, at least 9:1, at least 10:1, at least 20:1, at least 30:1, at least 40:1, at least 50:1 or at least 60:1. In a preferred embodiment, the relative weight ratio of the polyalkylene oxide to the opioid agonist is within the range of from 5:1 to 1:1, more preferably 4:1 to 2:1.

It has been surprisingly found that the release of both, the opioid agonist and the opioid antagonist, from the prolonged release matrix is substantially independent from the pH value of the release medium.

In a preferred embodiment, the pharmaceutical dosage form according to the invention is adapted for administration once daily. In another preferred embodiment, the pharmaceutical dosage form according to the invention is adapted for administration twice daily. In still another preferred embodiment, the pharmaceutical dosage form according to the invention is adapted for administration thrice daily, four times daily, five times daily, six times daily, or even more frequently.

For the purpose of the specification, “twice daily” means equal or nearly equal time intervals, i.e., about every 12 hours, or different time intervals, e.g., 8 and 16 hours or 10 and 14 hours, between the individual administrations.

For the purpose of the specification, “thrice daily” means equal or nearly equal time intervals, i.e., about every 8 hours, or different time intervals, e.g., 6, 6 and 12 hours; or 7, 7 and 10 hours, between the individual administrations.

According to the invention, in accordance with Ph. Eur., the in vitro release profile of the opioid agonist essentially corresponds to, i.e. is essentially identical to or at least resembling with the in vitro release profile of the opioid antagonist. For the purpose of the specification, “essentially corresponds” preferably means that opioid agonist and opioid antagonist are released according to same order kinetics, preferably both according to a prolonged release profile; preferably, however, “essentially corresponds” does not encompass pharmaceutical dosage forms where one of the opioid agonist and the opioid antagonist is released immediately, and the other one is released in a prolonged fashion.

It has been surprisingly found that an essentially identical or at least resembling in vitro release profile of opioid agonist and opioid antagonist can be achieved, though the pharmaceutical dosage form contains polyalkylene oxide, i.e. a hydrophilic polymer, which is necessary in order to achieve the substantially increased breaking strength of at least 300 N of the pharmaceutical dosage form. It is known that pharmaceutical dosage forms containing tilidin as opioid agonist and naloxon as opioid antagonist embedded in a hydrophilic matrix do not provide such an essentially identical or at least resembling in vitro release profile of the opioid agonist and the opioid antagonist (cf. EP 1 492 506, paragraph [0026]). Rather, these pharmaceutical dosage forms exhibit an in vitro release profile of the opioid agonist that substantially differs from the in vitro release profile of the opioid antagonist. As it is desirable to have an essentially identical or at least resembling in vitro release profile of both, the opioid agonist and the opioid antagonist, attempts have been made in the art to somehow approximate both in vitro release profiles. This could be achieved on the basis of hydrophobic matrix materials which, however, are typically not suitable for manufacturing pharmaceutical dosage forms having an increased breaking strength of at least 300 N. It has now been surprisingly found that the same can be achieved even on the basis of a hydrophilic matrix material, namely polyalkylene oxide, optionally in combination with other matrix polymers.

Preferably, at every point in time the in vitro release profile of the opioid agonist does absolutely not deviate by more than 10%, more preferably not more than 9%, still more preferably not more than 8%, yet more preferably not more than 7%, even more preferably not more than 6%, most preferably not more than 5% and in particular not more than 4% or not more than 3% from the in vitro release profile of the opioid antagonist. For example, if the pharmaceutical dosage form releases under in vitro conditions in accordance with Ph. Eur. 23% of the opioid antagonist 2 h after administration, it preferably releases 23±10% (=from 13% to 33%) of the opioid agonist 2 h after administration.

Preferably, the pharmaceutical dosage form according to the invention causes an at least partially delayed or prolonged release of opioid agonist and opioid antagonist.

Controlled or prolonged release is understood according to the invention preferably to mean a release profile in which the opioid agonist and the opioid antagonist is released over a relatively long period with reduced intake frequency with the purpose of extended therapeutic action of the opioid agonist. Preferably, the meaning of the term “prolonged release” is in accordance with the European guideline on the nomenclature of the release profile of pharmaceutical dosage forms (CHMP). This is achieved in particular with peroral administration. The expression “at least partially delayed or prolonged release” covers according to the invention any pharmaceutical dosage forms which ensure modified release of the opioid agonists and opioid antagonists contained therein. The pharmaceutical dosage forms preferably comprise coated or uncoated pharmaceutical dosage forms, which are produced with specific auxiliary substances, by particular processes or by a combination of the two possible options in order purposefully to change the release rate or location of release.

In the case of the pharmaceutical dosage forms according to the invention, the release profile of a controlled release form may be modified e.g. as follows: extended release, repeat action release, prolonged release and sustained release.

For the purpose of the specification “controlled release” preferably means a product in which the release of active compound over time is controlled by the type and composition of the formulation. For the purpose of the specification “extended release” preferably means a product in which the release of active compound is delayed for a finite lag time, after which release is unhindered. For the purpose of the specification “repeat action release” preferably means a product in which a first portion of active compound is released initially, followed by at least one further portion of active compound being released subsequently. For the purpose of the specification “prolonged release” preferably means a product in which the rate of release of active compound from the formulation after administration has been reduced over time, in order to maintain therapeutic activity, to reduce toxic effects, or for some other therapeutic purpose. For the purpose of the specification “sustained release” preferably means a way of formulating a medicine so that it is released into the body steadily, over a long period of time, thus reducing the dosing frequency. For further details, reference may be made, for example, to K. H. Bauer, Lehrbuch der Pharmazeutischen Technologie, 6th edition, WVG Stuttgart, 1999; and Eur. Ph.

The pharmaceutical dosage form according to the invention may comprise one or more opioid agonists and opioid antagonists at least in part in a further controlled release form, wherein controlled release may be achieved with the assistance of conventional materials and processes known to the person skilled in the art, for example by embedding the substances in a controlled release matrix or by applying one or more controlled release coatings. Substance release must, however, be controlled such that addition of delayed-release materials does not impair the necessary breaking strength. Controlled release from the pharmaceutical dosage form according to the invention is preferably achieved by embedding the opioid agonist and the opioid antagonist in a matrix. Preferably, the polyalkylene oxide serves as matrix material in combination with auxiliary substances also acting as matrix materials. The auxiliary substances acting as matrix materials control release. Matrix materials may, for example, be hydrophilic, gel-forming materials, from which release proceeds mainly by erosion and diffusion.

Preferably, the release profile is substantially matrix controlled, preferably by embedding the opioid agonist and the opioid antagonist in a matrix comprising the polyalkylene oxide and optionally, further matrix materials. Preferably, the release profile is not osmotically driven. Preferably, release kinetics is not zero order.

In preferred embodiments, in accordance with Ph. Eur., the in vitro release profile of the opioid agonist as well as the in vitro release profile of the opioid antagonist in each case complies with any same single one of the following release profiles R¹ to R⁵⁰:

| % | R¹ | R² | R³ | R⁴ | R⁵ | R⁶ | R⁷ | R⁸ | R⁹ | R¹⁰ |
|---|---|---|---|---|---|---|---|---|---|---|
| 1 h | 30 ± 28 | 30 ± 26 | 30 ± 24 | 30 ± 22 | 30 ± 20 | 30 ± 18 | 30 ± 16 | 30 ± 14 | 30 ± 12 | 30 ± 10 |
| 2 h | 45 ± 40 | 45 ± 38 | 45 ± 36 | 45 ± 34 | 45 ± 32 | 45 ± 30 | 45 ± 28 | 45 ± 26 | 45 ± 24 | 45 ± 24 |
| 4 h | 60 ± 35 | 60 ± 33 | 60 ± 31 | 60 ± 29 | 60 ± 27 | 60 ± 25 | 60 ± 23 | 60 ± 21 | 60 ± 19 | 60 ± 17 |
| 6 h | 70 ± 30 | 70 ± 28 | 70 ± 25 | 70 ± 23 | 70 ± 21 | 70 ± 19 | 70 ± 17 | 70 ± 15 | 70 ± 13 | 70 ± 11 |
| 8 h | ≧60 | 85 ± 13 | 85 ± 12 | 85 ± 11 | 85 ± 10 | 85 ± 9 | 85 ± 8 | 85 ± 7 | 85 ± 6 | 85 ± 5 |
| 10 h | ≧70 | ≧72 | ≧74 | ≧76 | ≧78 | ≧80 | ≧82 | ≧84 | ≧86 | ≧88 |
| 12 h | ≧80 | ≧82 | ≧84 | ≧86 | ≧88 | ≧90 | ≧92 | ≧94 | ≧96 | ≧98 |
| % | R¹¹ | R¹² | R¹³ | R¹⁴ | R¹⁵ | R¹⁶ | R¹⁷ | R¹⁸ | R¹⁹ | R²⁰ |
| 1 h | 40 ± 38 | 40 ± 36 | 40 ± 34 | 40 ± 32 | 40 ± 30 | 40 ± 28 | 40 ± 26 | 40 ± 24 | 40 ± 22 | 40 ± 20 |
| 2 h | 55 ± 43 | 55 ± 41 | 55 ± 39 | 55 ± 37 | 55 ± 35 | 55 ± 33 | 55 ± 31 | 55 ± 29 | 55 ± 27 | 55 ± 25 |
| 4 h | 70 ± 28 | 70 ± 26 | 70 ± 24 | 70 ± 22 | 70 ± 20 | 70 ± 18 | 70 ± 16 | 70 ± 14 | 70 ± 12 | 70 ± 10 |
| 6 h | 80 ± 20 | 80 ± 18 | 80 ± 16 | 80 ± 15 | 80 ± 14 | 80 ± 13 | 80 ± 12 | 80 ± 11 | 80 ± 10 | 80 ± 9 |
| 8 h | ≧80 | 90 ± 8 | 90 ± 8 | 90 ± 7 | 90 ± 7 | 90 ± 6 | 90 ± 6 | 90 ± 5 | 90 ± 5 | 90 ± 4 |
| 10 h | ≧85 | ≧87 | ≧89 | ≧90 | ≧90 | ≧91 | ≧91 | ≧92 | ≧92 | ≧92 |
| 12 h | ≧90 | ≧91 | ≧91 | ≧91 | ≧92 | ≧92 | ≧92 | ≧93 | ≧93 | ≧93 |
| % | R²¹ | R²² | R²³ | R²⁴ | R²⁵ | R²⁶ | R²⁷ | R²⁸ | R²⁹ | R³⁰ |
| 1 h | 20 ± 18 | 20 ± 16 | 20 ± 14 | 20 ± 13 | 20 ± 12 | 20 ± 11 | 20 ± 10 | 20 ± 9 | 20 ± 8 | 20 ± 7 |
| 2 h | 35 ± 33 | 35 ± 31 | 35 ± 30 | 35 ± 29 | 35 ± 27 | 35 ± 25 | 35 ± 23 | 35 ± 21 | 35 ± 19 | 35 ± 17 |
| 4 h | 50 ± 48 | 50 ± 46 | 50 ± 44 | 50 ± 42 | 50 ± 40 | 50 ± 38 | 50 ± 36 | 50 ± 34 | 50 ± 32 | 50 ± 31 |
| 6 h | 60 ± 38 | 60 ± 36 | 60 ± 34 | 60 ± 32 | 60 ± 30 | 60 ± 28 | 60 ± 26 | 60 ± 24 | 60 ± 22 | 60 ± 20 |
| 8 h | ≧60 | 70 ± 28 | 70 ± 26 | 70 ± 24 | 70 ± 22 | 70 ± 20 | 70 ± 18 | 70 ± 16 | 70 ± 14 | 70 ± 12 |
| 10 h | ≧70 | ≧72 | ≧74 | ≧76 | ≧78 | ≧80 | ≧82 | ≧84 | ≧86 | ≧88 |
| 12 h | ≧80 | ≧82 | ≧84 | ≧86 | ≧88 | ≧90 | ≧91 | ≧92 | ≧93 | ≧93 |
| % | R³¹ | R³² | R³³ | R³⁴ | R³⁵ | R³⁶ | R³⁷ | R³⁸ | R³⁹ | R⁴⁰ |
| 1 h | 8 ± 7 | 8 ± 6 | 8 ± 5 | 8 ± 4 | 13 ± 12 | 13 ± 10 | 13 ± 8 | 13 ± 6 | 18 ± 17 | 18 ± 14 |
| 2 h | 15 ± 14 | 15 ± 11 | 15 ± 8 | 15 ± 5 | 24 ± 23 | 24 ± 18 | 24 ± 13 | 24 ± 8 | 33 ± 32 | 33 ± 24 |
| 4 h | 30 ± 29 | 30 ± 22 | 30 ± 15 | 30 ± 8 | 38 ± 37 | 38 ± 28 | 38 ± 18 | 38 ± 8 | 55 ± 34 | 55 ± 26 |
| 6 h | 50 ± 49 | 50 ± 37 | 50 ± 25 | 50 ± 13 | 60 ± 39 | 60 ± 29 | 60 ± 19 | 60 ± 9 | 70 ± 29 | 70 ± 22 |
| 8 h | 65 ± 34 | 65 ± 26 | 65 ± 18 | 65 ± 10 | 75 ± 24 | 75 ± 18 | 75 ± 12 | 75 ± 6 | 83 ± 16 | 83 ± 13 |
| 10 h | 85 ± 14 | 85 ± 11 | 85 ± 8 | 85 ± 5 | 87 ± 12 | 87 ± 10 | 87 ± 8 | 87 ± 6 | 90 ± 9 | 90 ± 8 |
| 12 h | >95 | >95 | >95 | >95 | >95 | >95 | >95 | >95 | >95 | >95 |
| % | R⁴¹ | R⁴² | R⁴³ | R⁴⁴ | R⁴⁵ | R⁴⁶ | R⁴⁷ | R⁴⁸ | R⁴⁹ | R⁵⁰ |
| 1 h | 18 ± 11 | 18 ± 8 | 25 ± 24 | 25 ± 18 | 25 ± 12 | 25 ± 6 | 40 ± 39 | 40 ± 29 | 40 ± 19 | 40 ± 9 |
| 2 h | 33 ± 16 | 33 ± 8 | 45 ± 44 | 45 ± 33 | 45 ± 22 | 45 ± 11 | 63 ± 26 | 63 ± 20 | 63 ± 14 | 63 ± 8 |
| 4 h | 55 ± 18 | 55 ± 10 | 70 ± 29 | 70 ± 22 | 70 ± 15 | 70 ± 8 | 85 ± 14 | 85 ± 12 | 85 ± 10 | 85 ± 8 |
| 6 h | 70 ± 15 | 70 ± 8 | 83 ± 16 | 83 ± 13 | 83 ± 10 | 83 ± 7 | 90 ± 9 | 90 ± 8 | 90 ± 7 | 90 ± 6 |
| 8 h | 83 ± 10 | 83 ± 7 | 92 ± 7 | 92 ± 6 | 92 ± 6 | 92 ± 5 | 92 ± 7 | 92 ± 7 | 92 ± 6 | 92 ± 6 |
| 10 h | 90 ± 7 | 90 ± 6 | 94 ± 6 | 94 ± 6 | 94 ± 5 | 94 ± 5 | 94 ± 6 | 94 ± 6 | 94 ± 5 | 94 ± 5 |
| 12 h | >95 | >95 | >95 | >95 | >95 | >95 | >95 | >95 | >95 | >95 |

Suitable in vitro conditions are known to the skilled artisan. In this regard it can be referred to, e.g., the Ph. Eur. Preferably, the in vitro release profile is measured under the following conditions: 600 ml of blank FeSSIF (pH 5.0) at temperature of 37° C. with sinker (type 1 or 2). The rotation speed of the paddle is adjusted to 150/min. The pharmacologically active ingredient is detected by means of a spectrometric measurement with a wavelength of 218 nm.

Preferably, the release profile of the pharmaceutical dosage form according to the present invention is stable upon storage, preferably upon storage at elevated temperature, e.g. 40° C., for 3 months in sealed containers. In this regard “stable” means that when comparing the initial release profile with the release profile after storage, at any given time point the release profiles deviate from one another absolutely by not more than 20%, more preferably not more than 15%, still more preferably not more than 10%, yet more preferably not more than 7.5%, most preferably not more than 5.0% and in particular not more than 2.5%.

Preferably, the pharmaceutical dosage form according to the invention is monolithic. In this regard, the pharmaceutical dosage form does preferably not comprise a matrix and melt-extruded particulates comprising the opioid-agonist, wherein the melt-extruded particulates are present as a discontinuous phase in said matrix. Preferably, the pharmaceutical dosage form is a monolithic mass. The pharmaceutical dosage form is preferably prepared by hot-melt extrusion. The melt extruded strands are preferably cut into monoliths, which are then preferably formed into tablets. In this regard, the term “tablets” is preferably not to be understood as pharmaceutical dosage forms being made by compression of powder or granules (compressi) but rather, as shaped extrudates.

The pharmaceutical dosage form according to the invention comprises a polyalkylene oxide having a weight average molecular weight M_{w} of at least 200,000 g/mol, preferably at least 500,000 g/mol, more preferably at least 750,000 g/mol, still more preferably at least 1,000,000 g/mol, yet more preferably at least 1,500,000 g/mol, most preferably at least 2,000,000 g/mol and in particular within the range of from 500,000 to 15,000,000 g/mol.

Preferably, the polyalkylene oxide is selected from the group consisting of polymethylene oxide, polyethylene oxide and polypropylene oxide, the copolymers and mixtures thereof.

Polyalkylene oxide may comprise a single polyalkylene oxide having a particular average molecular weight, or a mixture (blend) of different polymers, such as two, three, four or five polymers, e.g., polymers of the same chemical nature but different average molecular weight, polymers of different chemical nature but same average molecular weight, or polymers of different chemical nature as well as different molecular weight.

For the purpose of the specification, a polyalkylene glycol has a molecular weight of up to 20,000 g/mol whereas a polyalkylene oxide has a molecular weight of more than 20,000 g/mol. In a preferred embodiment, the weight average over all molecular weights of all polyalkylene oxides that are contained in the pharmaceutical dosage form is at least 200,000 g/mol. Thus, polyalkylene glycols, if any, are preferably not taken into consideration when determining the weight average molecular weight of polyalkylene oxide.

Preferably, the content of the polyalkylene oxide is within the range of from 20 to 99 wt.-%, more preferably 25 to 95 wt.-%, still more preferably 30 to 90 wt.-%, yet more preferably 30 to 85 wt.-%, most preferably 30 to 80 wt.-% and in particular 30 to 75 wt.-%, based on the total weight of the pharmaceutical dosage form. In a preferred embodiment, the content of the polyalkylene oxide is at least 10 wt.-%, more preferably at least 15 wt.-%, still more preferably at least 20 wt.-%, yet more preferably at least 25 wt.-% and in particular at least 30 wt.-%, based on the total weight of the pharmaceutical dosage form.

In a preferred embodiment, the overall content of polyalkylene oxide is within the range of 25±20 wt.-%, more preferably 25±15 wt.-%, most preferably 25±10 wt.-%, and in particular 25±5 wt.-%. In another preferred embodiment, the overall content of polyalkylene oxide is within the range of 35±20 wt.-%, more preferably 35±15 wt.-%, most preferably 35±10 wt.-%, and in particular 35±5 wt.-%. In still another preferred embodiment, the overall content of polyalkylene oxide is within the range of 45±20 wt.-%, more preferably 45±15 wt.-%, most preferably 45±10 wt.-%, and in particular 45±5 wt.-%. In yet another preferred embodiment, the overall content of polyalkylene oxide is within the range of 55±20 wt.-%, more preferably 55±15 wt.-%, most preferably 55±10 wt.-%, and in particular 55±5 wt.-%. In a further preferred embodiment, the overall content of polyalkylene oxide is within the range of 65±20 wt.-%, more preferably 65±15 wt.-%, most preferably 65±10 wt.-%, and in particular 65±5 wt.-%. In still a further preferred embodiment, the overall content of polyalkylene oxide is within the range of 75±20 wt.-%, more preferably 75±15 wt.-%, most preferably 75±10 wt.-%, and in particular 75±5 wt.-%. In a still further a preferred embodiment, the overall content of polyalkylene oxide is within the range of 80±15 wt.-%, more preferably 80±10 wt.-%, and most preferably 80±5 wt.-%. In yet a further preferred embodiment, the overall content of polyalkylene oxide is within the range of 90±9 wt.-%, more preferably 90±5 wt.-%, and most preferably 90±3 wt.-%.

In a preferred embodiment, the polyalkylene oxide is homogeneously distributed in the pharmaceutical dosage form according to the invention. Preferably, the polyalkylene oxide forms a matrix in which the opioid agonist and the opioid antagonist are embedded. In a particularly preferred embodiment, the opioid agonist, the opioid antagonist and the polyalkylene oxide are intimately homogeneously distributed in the pharmaceutical dosage form so that the pharmaceutical dosage form does not contain any segments where either opioid agonist is present in the absence of opioid antagonist and/or polyalkylene oxide, or where opioid antagonist is present in the absence of opioid agonist and/or polyalkylene oxide or where polyalkylene oxide is present in the absence of opioid agonist and/or opioid antagonist.

When the pharmaceutical dosage form is film coated, the polyalkylene oxide is preferably homogeneously distributed in the core of the pharmaceutical dosage form, i.e. the film coating preferably does not contain polyalkylene oxide, but may e.g. contain polyethylene glycol. Nonetheless, the film coating as such may of course contain one or more polymers, which however, preferably differ from the polyalkylene oxide contained in the core.

The polyalkylene oxide may be combined with one or more different polymers selected from the group consisting of polyalkylene oxide, preferably polymethylene oxide, polyethylene oxide, polypropylene oxide; polyethylene, polypropylene, polyvinyl chloride, polycarbonate, polystyrene, polyvinylpyrrolidone, poly(hydroxy fatty acids), such as for example poly(3-hydroxybutyrate-co-3-hydroxyvalerate) (Biopol®), poly(hydroxyvaleric acid); polycaprolactone, polyvinyl alcohol, polyesteramide, polyethylene succinate, polylactone, polyglycolide, polyurethane, polyamide, polylactide, polyacetal (for example polysaccharides optionally with modified side chains), polylactide/glycolide, polylactone, polyglycolide, polyorthoester, polyanhydride, block polymers of polyethylene glycol and polybutylene terephthalate (Polyactive®), polyanhydride (Polifeprosan), copolymers thereof, block-copolymers thereof, and mixtures of at least two of the stated polymers, or other polymers with the above characteristics.

Preferably, the molecular weight dispersity M_{w}/Mₙ of polyalkylene oxide is within the range of 2.5±2.0, more preferably 2.5±1.5, still more preferably 2.5±1.0, yet more preferably 2.5±0.8, most preferably 2.5±0.6, and in particular 2.5±0.4.

The polyalkylene oxide preferably has a viscosity at 25° C. of 30 to 17,600 cP, more preferably 55 to 17,600 cP, still more preferably 600 to 17,600 cP and most preferably 4,500 to 17,600 cP, measured in a 5 wt.-% aqueous solution using a model RVF Brookfield viscosimeter (spindle no. 2/rotational speed 2 rpm); of 400 to 4,000 cP, more preferably 400 to 800 cP or 2,000 to 4,000 cP, measured on a 2 wt.-% aqueous solution using the stated viscosimeter (spindle no. 1 or 3/rotational speed 10 rpm); or of 1,650 to 10,000 cP, more preferably 1,650 to 5,500 cP, 5,500 to 7,500 cP or 7,500 to 10,000 cP, measured on a 1 wt.-% aqueous solution using the stated viscosimeter (spindle no. 2/rotational speed 2 rpm).

In a preferred embodiment, the prolonged release matrix comprises an additional matrix polymer.

In a preferred embodiment according to the invention, the polyalkylene oxide having a weight average molecular weight of at least 200,000 g/mol is combined with at least one further polymer, preferably but not necessarily also having a weight average molecular weight (M_{w}) of at least 200,000 g/mol, selected from the group consisting of polyethylene, polypropylene, polyvinyl chloride, polycarbonate, polystyrene, poly(hydroxy fatty acids), polycaprolactone, polyvinyl alcohol, polyesteramide, polyethylene succinate, polylactone, polyglycolide, polyurethane, polyvinylpyrrolidone, polyamide, polylactide, polylactide/glycolide, polylactone, polyglycolide, polyorthoester, polyanhydride, block polymers of polyethylene glycol and polybutylene terephthalate, polyanhydride, polyacetal, cellulose esters, cellulose ethers and copolymers thereof. Cellulose esters and cellulose ethers are particularly preferred, e.g. methylcellulose, ethylcellulose, hydroxymethylcellulose, hydroxyethylcellulose, hydroxypropylcellulose hydroxypropylmethylcellulose, carboxymethylcellulose, and the like.

In a preferred embodiment, said further polymer is neither a polyalkylene oxide nor a polyalkylene glycol. Nonetheless, the pharmaceutical dosage form may contain polyalkylene glycol, e.g. as plasticizer, but then, the pharmaceutical dosage form preferably is a ternary mixture of polymers: polyalkylene oxide+further polymer+plasticizer.

In a particularly preferred embodiment, said further polymer is a hydrophilic cellulose ester or cellulose ether, preferably hydroxypropylmethylcellulose (HPMC), hydroxypropylcellulose (HPC) or hydroxyethylcellulose (HEC), preferably having an average viscosity (preferably measured by capillary viscosimetry or rotational viscosimetry) of 1,000 to 150,000 mPas, more preferably 3,000 to 150,000. In a preferred embodiment, the average viscosity is within the range of 110,000±50,000 mPas, more preferably 110,000±40,000 mPas, still more preferably 110,000±30,000 mPas, most preferably 110,000±20,000 mPas, and in particular 100,000±10,000 mPas.

In a preferred embodiment the relative weight ratio of said polyalkylene oxide and said further polymer is within the range of from 20:1 to 1:20, more preferably 15:1 to 1:10, still more preferably 10:1 to 1:5, yet more preferably 8:1 to 1:1, most preferably 8:1 to 2:1 and in particular 8:1 to 3:1. In a preferred embodiment, the relative weight ratio of said polyalkylene oxide and said further polymer is within the range of from 10:1 to 5:1, more preferably 8:1 to 5:1, most preferably 7:1 to 5:1. In another preferred embodiment, the relative weight ratio of said polyalkylene oxide and said further polymer is within the range of from 5:1 to 1:1, more preferably 4:1 to 1:1, most preferably 3:1 to 1:1.

Preferably, the content of said further polymer amounts to 0.5 to 25 wt.-%, more preferably 1.0 to 20 wt.-%, still more preferably 2.0 to 22.5 wt.-%, yet more preferably 3.0 to 20 wt.-% and most preferably 4.0 to 17.5 wt.-% and in particular 5.0 to 15 wt.-%, based on the total weight of the pharmaceutical dosage form.

In a preferred embodiment, the further polymer is a cellulose ester or cellulose ether, preferably HPMC, having a content within the range of 10±8 wt.-%, more preferably 10±6 wt.-%, still more preferably 10±5 wt.-%, yet more preferably 10±4 wt.-%, most preferably 10±3 wt.-%, and in particular 10±2 wt.-%, based on the total weight of the pharmaceutical dosage form.

In another preferred embodiment, the further polymer is a cellulose ester or cellulose ether, preferably HPMC, having a content within the range of 15±8 wt.-%, more preferably 15±6 wt.-%, still more preferably 15±5 wt.-%, yet more preferably 15±4 wt.-%, most preferably 15±3 wt.-%, and in particular 15±2 wt.-%, based on the total weight of the pharmaceutical dosage form.

In still another preferred embodiment, the further polymer is a cellulose ester or cellulose ether, preferably HPMC, having a content within the range of 18±8 wt.-%, more preferably 18±6 wt.-%, still more preferably 18±5 wt.-%, yet more preferably 18±4 wt.-%, most preferably 18±3 wt.-%, and in particular 18±2 wt.-%, based on the total weight of the pharmaceutical dosage form.

All polymers are preferably employed as powders. They can be soluble in water.

Preferably, the pharmaceutical dosage form according to the invention is thermoformed, more preferably hot-melt extruded, although also other methods of thermoforming may be used in order to manufacture the pharmaceutical dosage form according to the invention, such as press-molding at elevated temperature or heating of tablets that were manufactured by conventional compression in a first step and then heated above the softening temperature of the polymer in the tablet in a second step to form hard tablets. In this regards, thermoforming means forming or molding of a mass after the application of heat. In a preferred embodiment, the pharmaceutical dosage form is thermoformed by hot-melt extrusion.

In a preferred embodiment, the pharmaceutical dosage form according to the invention has an overall density within the range of 1.19±0.30 g/cm³, more preferably 1.19±0.25 g/cm³, still more preferably 1.19±0.20 g/cm³, yet more preferably 1.19±0.15 g/cm³, most preferably 1.19±0.10 g/cm³, and in particular 1.19±0.05 g/cm³. Preferably, the overall density of the pharmaceutical dosage form according to the invention is 1.17±0.02 g/cm³, 1.19±0.02 g/cm³ or 1.21±0.02 g/cm³. Methods for measuring the density of a pharmaceutical dosage form are known to a person skilled in the art. The overall density of a pharmaceutical dosage form can for example be determined by means of the mercury porosimetry method or the helium pycnometer method as described in Ph. Eur.

In a preferred embodiment, the pharmaceutical dosage form has a total weight within the range of 100±75 mg, more preferably 100±50 mg, most preferably 100±25 mg. In another preferred embodiment, the pharmaceutical dosage form has a total weight within the range of 200±75 mg, more preferably 200±50 mg, most preferably 200±25 mg. In another preferred embodiment, the pharmaceutical dosage form has a total weight within the range of 250±75 mg, more preferably 250±50 mg, most preferably 250±25 mg. In still another preferred embodiment, the pharmaceutical dosage form has a total weight within the range of 300±75 mg, more preferably 300±50 mg, most preferably 300±25 mg. In yet another preferred embodiment, the pharmaceutical dosage form has a total weight within the range of 400±75 mg, more preferably 400±50 mg, most preferably 400±25 mg.

In a preferred embodiment, the pharmaceutical dosage form has a total weight within the range of 500±250 mg, more preferably 500±200 mg, most preferably 500±150 mg. In another preferred embodiment, the pharmaceutical dosage form has a total weight within the range of 750±250 mg, more preferably 750±200 mg, most preferably 750±150 mg. In another preferred embodiment, the pharmaceutical dosage form has a total weight within the range of 1000±250 mg, more preferably 1000±200 mg, most preferably 1000±150 mg. In still another preferred embodiment, the pharmaceutical dosage form has a total weight within the range of 1250±250 mg, more preferably 1250±200 mg, most preferably 1250±150 mg.

The pharmaceutical dosage form according to the invention contains, as opioid agonist, preferably oxymorphone, oxycodone or hydromorphone. For the purpose of the specification, the term opioid agonist also includes the free base and the physiologically acceptable salts thereof.

According to the ATC index, opioid agonists (opioids) are divided into natural opium alkaloids, phenylpiperidine derivatives, diphenylpropylamine derivatives, benzomorphan derivatives, oripavine derivatives, morphinan derivatives and others. Examples of natural opium alkaloids are morphine, opium, hydromorphone, nicomorphine, oxycodone, dihydrocodeine, diamorphine, papavereturn, and codeine. Further opioid agonists are, for example, ethylmorphine, hydrocodone, oxymorphone, and the physiologically acceptable derivatives thereof or compounds, preferably the salts and solvates thereof, preferably the hydrochlorides thereof, physiologically acceptable enantiomers, stereoisomers, diastereomers and racemates and the physiologically acceptable derivatives thereof, preferably ethers, esters or amides.

Further preferred opioid agonists include N-(1-methyl-2-piperidinoethyl)-N-(2-pyridyl)propionamide, (1R,2R)-3-(3-dimethylamino-1-ethyl-2-methyl-propyl)phenol (tapentadol), (1R,2R,4S)-2-(dimethylamino)methyl-4-(p-fluorobenzyloxy)-1-(m-methoxyphenyl)cyclohexanol, (1R,2R)-3-(2-dimethylaminomethyl-cyclohexyl)phenol, (1S,2S)-3-(3-dimethylamino-1-ethyl-2-methyl-propyl)phenol, (2R,3R)-1-dimethylamino-3(3-methoxyphenyl)-2-methyl-pentan-3-ol, (1RS, 3RS,6RS)-6-dimethylaminomethyl-1-(3-methoxyphenyl)-cyclohexane-1,3-diol, preferably as racemate, 3-(2-dimethylaminomethyl-1-hydroxy-cyclohexyl)phenyl 2-(4-isobutyl-phenyl)-propionate, 3-(2-dimethylaminomethyl-1-hydroxy-cyclohexyl)phenyl 2-(6-methoxy-naphthalen-2-yl)propionate, 3-(2-dimethylaminomethyl-cyclohex-1-enyl)-phenyl 2-(4-isobutyl-phenyl)-propionate, 3-(2-dimethylaminomethyl-cyclohex-1-enyl)-phenyl 2-(6-methoxy-naphthalen-2-yl)propionate, (RR-SS)-2-acetoxy-4-trifluoromethyl-benzoic acid 3-(2-dimethylaminomethyl-1-hydroxy-cyclohexyl)-phenyl ester, (RR-SS)-2-hydroxy-4-trifluoromethyl-benzoic acid 3-(2-dimethylaminomethyl-1-hydroxy-cyclohexyl)-phenyl ester, (RR-SS)-4-chloro-2-hydroxy-benzoic acid 3-(2-dimethylaminomethyl-1-hydroxy-cyclohexyl)-phenyl ester, (RR-SS)-2-hydroxy-4-methyl-benzoic acid 3-(2-dimethylaminomethyl-1-hydroxy-cyclohexyl)-phenyl ester, (RR-SS)-2-hydroxy-4-methoxy-benzoic acid 3-(2-dimethylaminomethyl-1-hydroxy-cyclohexyl)-phenyl ester, (RR-SS)-2-hydroxy-5-nitro-benzoic acid 3-(2-dimethylaminomethyl-1-hydroxy-cyclohexyl)-phenyl ester, (RR-SS)-2′,4′-difluoro-3-hydroxy-biphenyl-4-carboxylic acid 3-(2-dimethylaminomethyl-1-hydroxy-cyclohexyl)-phenyl ester, 1,1-(3-dimethylamino-3-phenyl-pentamethylen)-6-fluor-1,3,4,9-tetrahydropyrano[3,4-b]indole, in particular its hemicitrate; 1,1-[3-dimethylamino-3-(2-thienyl)pentamethylen]-1,3,4,9-tetrahydropyrano[3,4-b]indole, in particular its citrate; and 1,1-[3-dimethylamino-3-(2-thienyl)pentamethylen]-1,3,4,9-tetrahydropyrano[3,4-b]-6-fluoro-indole, in particular its hemicitrate, and corresponding stereoisomeric compounds, in each case the corresponding derivatives thereof, physiologically acceptable enantiomers, stereoisomers, diastereomers and racemates and the physiologically acceptable derivatives thereof, e.g. ethers, esters or amides, and in each case the physiologically acceptable compounds thereof, in particular the salts thereof and solvates, e.g. hydrochlorides.

Particularly preferred opioid agonists include oxymorphone, oxycodone, hydromorphone, and the physiologically acceptable salts thereof. In a particularly preferred embodiment, the opioid agonist is oxycodone or a physiologically acceptable salt thereof.

The content of the opioid agonist in the pharmaceutical dosage form is not limited.

Preferably, the content of the opioid agonist is within the range of from 0.01 to 80 wt.-%, more preferably 0.1 to 50 wt.-%, still more preferably 1 to 25 wt.-%, based on the total weight of the pharmaceutical dosage form. In a preferred embodiment, the content of opioid agonist is within the range of from 1.0±0.9 wt.-%, more preferably 1.0±0.7 wt.-%, most preferably 1.0±0.5 wt.-%, and in particular 1.0±0.3 wt.-%, based on the total weight of the pharmaceutical dosage form. In another preferred embodiment, the content of opioid agonist is within the range of from 2.0±1.0 wt.-%, more preferably 2.0±0.7 wt.-%, most preferably 2.0±0.5 wt.-%, and in particular 2.0±0.3 wt.-%, based on the total weight of the pharmaceutical dosage form. In still another preferred embodiment, the content of opioid agonist is within the range of from 7±6 wt.-%, more preferably 7±5 wt.-%, still more preferably 5±4 wt.-%, 7±4 wt.-% or 9±4 wt.-%, most preferably 5±3 wt.-%, 7±3 wt.-% or 9±3 wt.-%, and in particular 5±2 wt.-%, 7±2 wt.-% or 9±2 wt.-%, based on the total weight of the pharmaceutical dosage form. In yet another preferred embodiment, the content of opioid agonist is within the range of from 11±10 wt.-%, more preferably 11±9 wt.-%, still more preferably 9±6 wt.-%, 11±6 wt.-%, 13±6 wt.-% or 15±6 wt.-%, most preferably 11±4 wt.-%, 13±4 wt.-% or 15±4 wt.-%, and in particular 11±2 wt.-%, 13±2 wt.-% or 15±2 wt.-%, based on the total weight of the pharmaceutical dosage form. In a further preferred embodiment, the content of opioid agonist is within the range of from 20±6 wt.-%, more preferably 20±5 wt.-%, still more preferably 20±4 wt.-%, most preferably 20±3 wt.-%, and in particular 20±2 wt.-%, based on the total weight of the pharmaceutical dosage form. In still a further preferred embodiment, the content of opioid agonist is within the range of from 25±6 wt.-%, more preferably 25±5 wt.-%, still more preferably 25±4 wt.-%, most preferably 25±3 wt.-%, and in particular 25±2 wt.-%, based on the total weight of the pharmaceutical dosage form. In yet a further preferred embodiment, the content of opioid agonist is within the range of from 30±6 wt.-%, more preferably 30±5 wt.-%, still more preferably 30±4 wt.-%, most preferably 30±3 wt.-%, and in particular 30±2 wt.-%, based on the total weight of the pharmaceutical dosage form.

Preferably, the total amount of the opioid agonist that is contained in the pharmaceutical dosage form is within the range of from 0.01 to 200 mg, more preferably 0.1 to 190 mg, still more preferably 1.0 to 180 mg, yet more preferably 1.5 to 160 mg, most preferably 2.0 to 100 mg and in particular 2.5 to 80 mg.

In a preferred embodiment, the opioid agonist is contained in the pharmaceutical dosage form in an amount of 7.5±5 mg, 10±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5, 140±5 mg, 150±5 mg, 160±5 mg, 170±5 mg or 180±5 mg. In another preferred embodiment, the opioid agonist is contained in the pharmaceutical dosage form in an amount of 5±2.5 mg, 7.5±2.5 mg, 10±2.5 mg, 15±2.5 mg, 20±2.5 mg, 25±2.5 mg, 30±2.5 mg, 35±2.5 mg, 40±2.5 mg, 45±2.5 mg, 50±2.5 mg, 55±2.5 mg, 60±2.5 mg, 65±2.5 mg, 70±2.5 mg, 75±2.5 mg, 80±2.5 mg, 85±2.5 mg, 90±2.5 mg, 95±2.5 mg, 100±2.5 mg, 105±2.5 mg, 110±2.5 mg, 115±2.5 mg, 120±2.5 mg, 125±2.5 mg, 130±2.5 mg, 135±2.5 mg, 140±2.5 mg, 145±2.5 mg, 150±2.5 mg, 155±2.5 mg, 160±2.5 mg, 165±2.5 mg, 170±2.5 mg, 175±2.5 mg or 180±2.5 mg.

In a preferred embodiment, opioid agonist is oxymorphone, preferably its HCl salt, and the pharmaceutical dosage form is adapted for administration twice daily. In this embodiment, opioid agonist is preferably contained in the pharmaceutical dosage form in an amount of from 5 to 60 mg. In another particularly preferred embodiment, the opioid agonist is oxymorphone, preferably its HCl salt, and the pharmaceutical dosage form is adapted for administration once daily. In this embodiment, opioid agonist is preferably contained in the pharmaceutical dosage form in an amount of from 10 to 100 mg.

In another preferred embodiment, opioid agonist is oxycodone, preferably its HCl salt, and the pharmaceutical dosage form is adapted for administration twice daily. In this embodiment, opioid agonist is preferably contained in the pharmaceutical dosage form in an amount of from 3 to 180 mg, preferably 5 to 80 mg, more preferably 150 to 180 mg or 80 to 100 mg or 50 to 70 mg or 45 to 25 mg or 10 to 13 mg and most preferably 5 mg, 7 mg, 10 mg, 20 mg, 35 mg, 40 mg, 60 mg, 90 mg, 160 mg or 177 mg. In another particularly preferred embodiment, the opioid agonist is oxycodone, preferably its HCl salt, and the pharmaceutical dosage form is adapted for administration once daily. In this embodiment, opioid agonist is preferably contained in the pharmaceutical dosage form in an amount of from 3 to 320 mg.

In still another particularly preferred embodiment, opioid agonist is hydromorphone, preferably its HCl, and the pharmaceutical dosage form is adapted for administration twice daily. In this embodiment, opioid agonist is preferably contained in the pharmaceutical dosage form in an amount of from 2 to 52 mg, preferably 3 to 40 mg and more preferably 3 to 30 mg. In another particularly preferred embodiment, opioid agonist is hydromorphone, preferably its HCl salt, and the pharmaceutical dosage form is adapted for administration once daily. In this embodiment, opioid agonist is preferably contained in the pharmaceutical dosage form in an amount of from 3 to 104 mg.

The pharmaceutical dosage form according to the invention is characterized by excellent storage stability. Preferably, after storage for 4 weeks at 40° C. and 75% rel. humidity, the content of opioid agonist and opioid antagonist in each case amounts to at least 90%, more preferably at least 91%, still more preferably at least 92%, yet more preferably at least 93%, most preferably at least 94% and in particular at least 95%, of its original content before storage. Suitable methods for measuring the content of the opioid agonist and opioid antagonist in the pharmaceutical dosage form are known to the skilled artisan. In this regard it is referred to the Eur. Ph. or the USP, especially to reversed phase HPLC analysis. Preferably, the pharmaceutical dosage form is stored in closed, preferably sealed containers, most preferably being equipped with an oxygen scavenger, in particular with an oxygen scavenger that is effective even at low relative humidity.

In a preferred embodiment, after oral administration of the pharmaceutical dosage form according to the invention, in vivo the average peak plasma level (Cₘₐₓ) of the opioid agonist is on average reached after tₘₐₓ 3.0±2.5 h, more preferably after tₘₐₓ 3.0±2.0 h, still more preferably after tₘₐₓ 3.0±1.5 h, most preferably after tₘₐₓ 3.0±1.0 h and in particular after tₘₐₓ 3.0±0.5 h. In a preferred embodiment, after oral administration of the pharmaceutical dosage form according to the invention, in vivo the average peak plasma level (Cₘₐₓ) of the opioid agonist is on average reached after tₘₐₓ 4.0±2.5 h, more preferably after tₘₐₓ 4.0±2.0 h, still more preferably after tₘₐₓ 4.0±1.5 h, most preferably after tₘₐₓ 4.0±1.0 h and in particular after tₘₐₓ 4.0±0.5 h. In another preferred embodiment, after oral administration of the pharmaceutical dosage form according to the invention, in vivo the average peak plasma level (Cₘₐₓ) of the opioid agonist is on average reached after tₘₐₓ 5.0±2.5 h, more preferably after tₘₐₓ 5.0±2.0 h, still more preferably after tₘₐₓ 5.0±1.5 h, most preferably after tₘₐₓ 5.0±1.0 h and in particular after tₘₐₓ 5.0±0.5 h. In still another preferred embodiment, after oral administration of the pharmaceutical dosage form according to the invention, in vivo the average peak plasma level (Cₘₐₓ) of the opioid agonist is on average reached after tₘₐₓ 6.0±2.5 h, more preferably after tₘₐₓ 6.0±2.0 h, still more preferably after tₘₐₓ 6.0±1.5 h, most preferably after tₘₐₓ 6.0±1.0 h and in particular after tₘₐₓ 6.0±0.5 h.

In a preferred embodiment, the average value for t_{1/2} of the opioid agonist after oral administration of the pharmaceutical dosage form according to the invention in vivo is 3.0±2.5 h, more preferably 3.0±2.0 h, still more preferably 3.0±1.5 h, most preferably 3.0±1.0 h, and in particular 3.0±0.5 h. In a preferred embodiment, the average value for t_{1/2} of the opioid agonist after oral administration of the pharmaceutical dosage form according to the invention in vivo is 4.0±2.5 h, more preferably 4.0±2.0 h, still more preferably 4.0±1.5 h, most preferably 4.0±1.0 h, and in particular 4.0±0.5 h. In another preferred embodiment, the average value for t_{1/2} of the opioid agonist after oral administration of the pharmaceutical dosage form according to the invention in vivo is preferably 5.0±2.5 h, more preferably 5.0±2.0 h, still more preferably 5.0±1.5 h, most preferably 5.0±1.0 h, and in particular 5.0±0.5 h. In still another preferred embodiment, the average value for t_{1/2} of the opioid agonist after oral administration of the pharmaceutical dosage form according to the invention in vivo is preferably 6.0±2.5 h, more preferably 6.0±2.0 h, still more preferably 6.0±1.5 h, most preferably 6.0±1.0 h, and in particular 6.0±0.5 h.

Preferably, Cₘₐₓ of the opioid agonist does not exceed 0.01 ng/ml, or 0.05 ng/ml, or 0.1 ng/ml, or 0.5 ng/ml, or 1.0 ng/ml, or 2.5 ng/ml, or 5 ng/ml, or 10 ng/ml, or 20 ng/ml, or 30 ng/ml, or 40 ng/ml, or 50 ng/ml, or 75 ng/ml, or 100 ng/ml, or 150 ng/ml, or 200 ng/ml, or 250 ng/ml, or 300 ng/ml, or 350 ng/ml, or 400 ng/ml, or 450 ng/ml, or 500 ng/ml, or 750 ng/ml, or 1000 ng/ml.

In a preferred embodiment, the opioid antagonist is selected from the group consisting of naltrexone, naloxone and its analogues such as naltrexol, naltrexamine and naloxol derivatives, nalmefene, cyclazacine, levallorphan, nalmefene, nalide, nalmexone, nalorphine, naluphine, pharmaceutically acceptable salts thereof and mixtures thereof.

Opioid antagonists that are not or only poorly bioavailable upon oral administration, but much better bioavailable upon parenteral administration, are particularly preferred.

Opioid antagonists suitable for a given opioid agonist are known to the person skilled in the art and may be present as such or in the form of corresponding derivatives, in particular esters or ethers, or in each case in the form of corresponding physiologically acceptable compounds, in particular in the form of the salts or solvates thereof. The pharmaceutical dosage form according to the invention preferably contains an opioid antagonist selected from the group consisting of naloxone, naltrexone, nalmefene, nalide, nalmexone, nalorphine or naluphine, in each case optionally in the form of a corresponding physiologically acceptable compound, in particular in the form of a base, a salt or solvate.

Naloxone and nalmexone as well as their physiologically acceptable salts are preferred opioid antagonists.

Naloxone is particularly preferred as opioid antagonist, preferably its hydrochloride, more preferably the dihydrate of the hydrochloride.

The content of the opioid antagonist in the pharmaceutical dosage form is not limited.

Preferably, the content of the opioid antagonist in the pharmaceutical dosage form according to the invention is such that it is at least sufficient to locally block the opioid receptors in the intestine thereby suppressing obstipation that would otherwise be induced by the opioid agonist. Preferably, however, the content of the opioid antagonist is increased to an amount sufficient to counter the effect of the opioid agonist when the pharmaceutical dosage form is tampered with, particularly by liquid extraction of the active ingredients and parenteral administration of the liquid extract. There is indication that the quantity needed for this effect is higher than the quantity needed for suppression of obstipation.

Preferably, the content of the opioid antagonist is within the range of from 0.01 to 80 wt.-%, more preferably 0.1 to 50 wt.-%, still more preferably 1 to 25 wt.-%, based on the total weight of the pharmaceutical dosage form. In a preferred embodiment, the content of opioid antagonist is within the range of from 1.0±0.9 wt.-%, more preferably 1.0±0.7 wt.-%, most preferably 1.0±4 wt.-%. In another preferred embodiment, the content of opioid antagonist is within the range of from 3.0±2.0 wt.-%, more preferably 3.0±1.0 wt.-%, most preferably 3.0±0.5 wt.-%. In still another preferred embodiment, the content of opioid antagonist is within the range of from 7±6 wt.-%, more preferably 7±5 wt.-%, still more preferably 5±4 wt.-%, 6±4 wt.-%, 7±4 wt.-% or 9±4 wt.-%, most preferably 5±3 wt.-%, 7±3 wt.-% or 9±3 wt.-%, and in particular 5±2 wt.-%, 7±2 wt.-% or 9±2 wt.-%, based on the total weight of the pharmaceutical dosage form. In yet another preferred embodiment, the content of opioid antagonist is within the range of from 11±10 wt.-%, more preferably 11±9 wt.-%, still more preferably 9±6 wt.-%, 11±6 wt.-%, 13±6 wt.-% or 15±6 wt.-%, most preferably 11±4 wt.-%, 13±4 wt.-% or 15±4 wt.-%, and in particular 11±2 wt.-%, 13±2 wt.-% or 15±2 wt.-%, based on the total weight of the pharmaceutical dosage form. In a further preferred embodiment, the content of opioid antagonist is within the range of from 20±6 wt.-%, more preferably 20±5 wt.-%, still more preferably 20±4 wt.-%, most preferably 20±3 wt.-%, and in particular 20±2 wt.-%, based on the total weight of the pharmaceutical dosage form.

Preferably, the total amount of the opioid antagonist that is contained in the pharmaceutical dosage form is within the range of from 0.01 to 200 mg, more preferably 0.1 to 190 mg, still more preferably 1.0 to 180 mg, yet more preferably 1.5 to 160 mg, most preferably 2.0 to 100 mg and in particular 2.5 to 80 mg.

In a preferred embodiment, the opioid antagonist is contained in the pharmaceutical dosage form in an amount of 1.0±0.5 mg, 2.0±1.0 mg, 3.0±1.0 mg, 4.0±1.0 mg, 5.0±1.0 mg, 7.5±5 mg, 8±5 mg, 10±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5, 140±5 mg, 150±5 mg, or 160±5 mg. In another preferred embodiment, the opioid antagonist is contained in the pharmaceutical dosage form in an amount of 3±2.5 mg, 5±2.5 mg, 7.5±2.5 mg, 10±2.5 mg, 15±2.5 mg, 18±2.5 mg, 20±2.5 mg, 25±2.5 mg, 30±2.5 mg, 35±2.5 mg, 40±2.5 mg, 45±2.5 mg, 50±2.5 mg, 55±2.5 mg, 60±2.5 mg, 65±2.5 mg, 70±2.5 mg, 75±2.5 mg, 80±2.5 mg, 85±2.5 mg, 87±2.5 mg, 90±2.5 mg, 95±2.5 mg, 100±2.5 mg, 105±2.5 mg, 110±2.5 mg, 115±2.5 mg, 120±2.5 mg, 125±2.5 mg, 130±2.5 mg, 135±2.5 mg, 140±2.5 mg, 145±2.5 mg, 150±2.5 mg, 155±2.5 mg, or 160±2.5 mg.

Preferably, the relative weight ratio of the opioid agonist and the opioid antagonist is within the range of from 20:1 to 1:5 or 10:1 to 1:20, more preferably 15:1 to 1:4 or 8:1 to 1:15, still more preferably 10:1 to 1:3 or 5:1 to 1:10, yet more preferably 5:1 to 1:2 or 3:1 to 1:7, even more preferably 3.5:1 to 1:1.5 or 2:1 to 1:5, most preferably 3:1 to 1:1 or 1:1 to 1:3.5, and in particular 2.5:1 to 1.5:1 or 1:1.5 to 1:2.5.

The purpose of the opioid antagonist that is contained in the pharmaceutical dosage form according to the invention is on the one hand associated with the tamper resistance of the pharmaceutical dosage form, especially when the pharmaceutical dosage form is administered by a non-prescribed route of administration, particularly intravenous administration of a liquid extract. Under these circumstances, the opioid antagonist preferably evolves its antagonizing effect thereby avoiding misuse of the opioid agonist. On the other hand, the purpose of the opioid antagonist is preferably to reduce undesired adverse events, particularly to counter obstipation that would be otherwise induced by the opioid agonist. This is achieved by locally blocking the pharmacological effect of the opioid agonist at the opioid receptors in the intestine upon prescribed oral administration of the pharmaceutical dosage form.

In a particularly preferred embodiment, the opioid antagonist is naloxone, preferably its HCl salt, and the pharmaceutical dosage form is adapted for administration twice daily. In this embodiment, the opioid antagonist is preferably contained in the pharmaceutical dosage form in an amount of from 1.0 to 100 mg, preferably 1.0 to 40 mg.

In a particularly preferred embodiment, the opioid agonist is oxycodone, preferably its hydrochloride, and the opioid antagonist is naloxone, preferably its hydrochloride. Preferred contents A¹ to A³⁰ of said opioid agonist and said opioid antagonist for this embodiment are summarized in the table here below:

| mg | A¹ | A² | A³ | A⁴ | A⁵ | A⁶ |
|---|---|---|---|---|---|---|
| opioid agonist | 5.0 ± 2.0 | 10 ± 2.0 | 15 ± 2.0 | 20 ± 2.0 | 25 ± 2.0 | 30 ± 2.0 |
| opioid antagonist | 2.5 ± 2.0 | 5.0 ± 4.5 | 7.5 ± 7.0 | 10 ± 9.5 | 12.5 ± 12.0 | 15 ± 14.5 |
| mg | A⁷ | A⁸ | A⁹ | A¹⁰ | A¹¹ | A¹² |
| opioid agonist | 35 ± 2.0 | 40 ± 2.0 | 50 ± 2.0 | 60 ± 2.0 | 70 ± 2.0 | 80 ± 2.0 |
| opioid antagonist | 17.5 ± 17.0 | 20 ± 19.5 | 25 ± 24.5 | 30 ± 29.5 | 35 ± 34.5 | 40 ± 39.5 |
| mg | A¹³ | A¹⁴ | A¹⁵ | A¹⁶ | A¹⁷ | A¹⁸ |
| opioid agonist | 5.0 ± 2.0 | 10 ± 2.0 | 15 ± 2.0 | 20 ± 2.0 | 25 ± 2.0 | 30 ± 2.0 |
| opioid antagonist | 2.5 ± 2.0 | 5.0 ± 2.0 | 7.5 ± 2.0 | 10 ± 2.0 | 12.5 ± 2.0 | 15 ± 2.0 |
| mg | A¹⁹ | A²⁰ | A²¹ | A²² | A²³ | A²⁴ |
| opioid agonist | 35 ± 2.0 | 40 ± 2.0 | 50 ± 2.0 | 60 ± 2.0 | 70 ± 2.0 | 80 ± 2.0 |
| opioid antagonist | 17.5 ± 2.0 | 20 ± 2.0 | 25 ± 2.0 | 30 ± 2.0 | 35 ± 2.0 | 40 ± 2.0 |
| mg | A²⁵ | A²⁶ | A²⁷ | A²⁸ | A²⁹ | A³⁰ |
| opioid agonist | 90 ± 2.0 | 120 ± 2.0 | 140 ± 2.0 | 160 ± 2.0 | 180 ± 2.0 | 190 ± 2.0 |
| opioid antagonist | 45 ± 2.0 | 60 ± 2.0 | 70 ± 2.0 | 80 ± 2.0 | 90 ± 2.0 | 95 ± 2.0 |

In another particularly preferred embodiment, the opioid agonist is hydromorphone, preferably its hydrochloride, and the opioid antagonist is naloxone, preferably its hydrochloride. According to this embodiment, the content of said opioid agonist is preferably in the range of from 0.5 to 30 mg, more preferably 1 to 20 mg, still more preferably 2 to 15 mg, most preferably 2.5 to 10 mg and in particular 3 to 5 mg. Further, according to this embodiment, the content of said opioid antagonist is preferably in the range of from 0.5 to 50 mg, more preferably 2 to 40 mg, still more preferably 3.5 to 30 mg, most preferably 5 to 20 mg and in particular 6 to 10 mg.

In a preferred embodiment, after oral administration of the pharmaceutical dosage form according to the invention, in vivo the average peak plasma level (Cₘₐₓ) of the opioid antagonist is on average reached after tₘₐₓ 3.0±2.5 h, more preferably after tₘₐₓ 3.0±2.0 h, still more preferably after tₘₐₓ 3.0±1.5 h, most preferably after tₘₐₓ 3.0±1.0 h and in particular after tₘₐₓ 3.0±0.5 h. In another preferred embodiment, after oral administration of the pharmaceutical dosage form according to the invention, in vivo the average peak plasma level (Cₘₐₓ) of the opioid antagonist is on average reached after tₘₐₓ 3.4±2.5 h, more preferably after tₘₐₓ 3.4±2.0 h, still more preferably after tₘₐₓ 3.4±1.5 h, most preferably after tₘₐₓ 3.4±1.0 h and in particular after tₘₐₓ 3.4±0.5 h. In still another preferred embodiment, after oral administration of the pharmaceutical dosage form according to the invention, in vivo the average peak plasma level (Cₘₐₓ) of the opioid antagonist is on average reached after tₘₐₓ 4.0±2.5 h, more preferably after tₘₐₓ 4.0±2.0 h, still more preferably after tₘₐₓ 4.0±1.5 h, most preferably after tₘₐₓ 4.0±1.0 h and in particular after tₘₐₓ 4.0±0.5 h. In yet another preferred embodiment, after oral administration of the pharmaceutical dosage form according to the invention, in vivo the average peak plasma level (Cₘₐₓ) of the opioid antagonist is on average reached after tₘₐₓ 5.0±2.5 h, more preferably after tₘₐₓ 5.0±2.0 h, still more preferably after tₘₐₓ 5.0±1.5 h, most preferably after tₘₐₓ 5.0±1.0 h and in particular after tₘₐₓ 5.0±0.5 h. In still another preferred embodiment, after oral administration of the pharmaceutical dosage form according to the invention, in vivo the average peak plasma level (Cₘₐₓ) of the opioid antagonist is on average reached after tₘₐₓ 6.0±2.5 h, more preferably after tₘₐₓ 6.0±2.0 h, still more preferably after tₘₐₓ 6.0±1.5 h, most preferably after tₘₐₓ 6.0±1.0 h and in particular after tₘₐₓ 6.0±0.5 h.

In a preferred embodiment, the average value for t_{1/2} of the opioid antagonist after oral administration of the pharmaceutical dosage form according to the invention in vivo is 4.0±2.5 h, more preferably 4.0±2.0 h, still more preferably 4.0±1.5 h, most preferably 4.0±1.0 h, and in particular 4.0±0.5 h. In another preferred embodiment, the average value for t_{1/2} of the opioid antagonist after oral administration of the pharmaceutical dosage form according to the invention in vivo is 4.3±2.5 h, more preferably 4.3±2.0 h, still more preferably 4.3±1.5 h, most preferably 4.3±1.0 h, and in particular 4.3±0.5 h. In still another preferred embodiment, the average value for t_{1/2} of the opioid antagonist after oral administration of the pharmaceutical dosage form according to the invention in vivo is preferably 5.0±2.5 h, more preferably 5.0±2.0 h, still more preferably 5.0±1.5 h, most preferably 5.0±1.0 h, and in particular 5.0±0.5 h. In yet another preferred embodiment, the average value for t_{1/2} of the opioid antagonist after oral administration of the pharmaceutical dosage form according to the invention in vivo is preferably 6.0±2.5 h, more preferably 6.0±2.0 h, still more preferably 6.0±1.5 h, most preferably 6.0±1.0 h, and in particular 6.0±0.5 h.

In a preferred embodiment, Cₘₐₓ of the opioid antagonist is below Cₘₐₓ of the opioid agonist. Preferably, Cₘₐₓ of the opioid antagonist is at most 90%, more preferably at most 80%, still more preferably at most 70%, yet more preferably at most 65%, even more preferably at most 60%, most preferably at most 55% and in particular at most 50% of Cₘₐₓ of the opioid agonist.

Preferably, Cₘₐₓ of the opioid antagonist does not exceed 0.01 ng/ml, or 0.05 ng/ml, or 0.1 ng/ml, or 0.5 ng/ml, or 1.0 ng/ml, or 2.5 ng/ml, or 5 ng/ml, or 10 ng/ml, or 20 ng/ml, or 30 ng/ml, or 40 ng/ml, or 50 ng/ml, or 75 ng/ml, or 100 ng/ml, or 150 ng/ml, or 200 ng/ml, or 250 ng/ml, or 300 ng/ml, or 350 ng/ml, or 400 ng/ml, or 450 ng/ml, or 500 ng/ml, or 750 ng/ml, or 1000 ng/ml.

Preferably, at any point in time during 8 h, more preferably 10 h, most preferably 12 h, after oral administration of the pharmaceutical dosage form, the plasma concentration of the opioid antagonist is below the plasma concentration of the opioid agonist. Preferably, at any point in time during 8 h, more preferably 10 h, most preferably 12 h, after oral administration of the pharmaceutical dosage form, the plasma concentration of the opioid antagonist is at most 90%, more preferably at most 80%, still more preferably at most 70%, yet more preferably at most 65%, even more preferably at most 60%, most preferably at most 55% and in particular at most 50% of the plasma concentration of the opioid agonist at the same point in time.

In a preferred embodiment, the pharmaceutical dosage form according to the invention contains no substances which irritate the nasal passages and/or pharynx, i.e. substances which, when administered via the nasal passages and/or pharynx, bring about a physical reaction which is either so unpleasant for the patient that he/she does not wish to or cannot continue administration, for example burning, or physiologically counteracts taking of the corresponding active compound, for example due to increased nasal secretion or sneezing. Further examples of substances which irritate the nasal passages and/or pharynx are those which cause burning, itching, urge to sneeze, increased formation of secretions or a combination of at least two of these stimuli. Corresponding substances and the quantities thereof which are conventionally to be used are known to the person skilled in the art. Some of the substances which irritate the nasal passages and/or pharynx are accordingly based on one or more constituents or one or more plant parts of a hot substance drug. Corresponding hot substance drugs are known per se to the person skilled in the art and are described, for example, in “Pharmazeutische Biologie—Drogen und ihre Inhaltsstoffe” by Prof. Dr. Hildebert Wagner, 2nd., revised edition, Gustav Fischer Verlag, Stuttgart-New York, 1982, pages 82 et seq. The corresponding description is hereby introduced as a reference and is deemed to be part of the disclosure.

The pharmaceutical dosage form according to the invention furthermore preferably contains no emetic. Emetics are known to the person skilled in the art and may be present as such or in the form of corresponding derivatives, in particular esters or ethers, or in each case in the form of corresponding physiologically acceptable compounds, in particular in the form of the salts or solvates thereof. The pharmaceutical dosage form according to the invention preferably contains no emetic based on one or more constituents of ipecacuanha (ipecac) root, for example based on the constituent emetine, as are, for example, described in “Pharmazeutische Biologie—Drogen und ihre Inhaltsstoffe” by Prof. Dr. Hildebert Wagner, 2nd, revised edition, Gustav Fischer Verlag, Stuttgart, New York, 1982. The corresponding literature description is hereby introduced as a reference and is deemed to be part of the disclosure. The pharmaceutical dosage form according to the invention preferably also contains no apomorphine as an emetic.

The pharmaceutical dosage form according to the invention preferably also contains no bitter substance. Bitter substances and the quantities effective for use may be found in US-2003/0064099 A1, the corresponding disclosure of which should be deemed to be the disclosure of the present application and is hereby introduced as a reference. Examples of bitter substances are aromatic oils, such as peppermint oil, eucalyptus oil, bitter almond oil, menthol, fruit aroma substances, aroma substances from lemons, oranges, limes, grapefruit or mixtures thereof, and/or denatonium benzoate.

The pharmaceutical dosage form according to the invention accordingly preferably contains neither substances which irritate the nasal passages and/or pharynx, nor emetics, nor bitter substances.

Preferably, the pharmaceutical dosage form according to the invention contains no neuroleptics, for example a compound selected from the group consisting of haloperidol, promethacine, fluphenazine, perphenazine, levomepromazine, thioridazine, perazine, chlorpromazine, chlorprothixine, zuclopenthixol, flupentixol, prothipendyl, zotepine, benperidol, pipamperone, melperone and bromperidol.

In other preferred embodiments, however, the pharmaceutical dosage form according to the invention does contain at least one of the aforementioned substances. In a preferred embodiment the pharmaceutical dosage form according to the invention may contain further abuse-complicating or abuse-preventing agents as auxiliary substances including aversive agents. Preferred aversive agents include but are not limited to:
(a) substances which irritate the nasal passages and/or pharynx (in the following also referred to as “component (a)”),
(b) viscosity-increasing agents and/or gelling agents (in the following also referred to as “component (b)”),
(c) emetics (in the following also referred to as “component (c)”),
(d) dyes (in the following also referred to as “component (d)”),
(e) bitter substances (in the following also referred to as “component (e)”), and/or
(f) surfactants (in the following also referred to as “component (f)”),
   and combinations of any of the foregoing, including (a)+(b), (a)+(c), (a)+(d), (a)+(e), (a)+(f); (b)+(c), (b)+(d), (b)+(e), (b)+(f); (c)+(d), (c)+(e), (c)+(f); (d)+(e), (d)+(f); and (e)+(f).

In a preferred embodiment, the dosage form according to the invention comprises component (a), i.e. a substance which irritates the nasal passages and/or pharynx.

Preferred components (a), i.e. substances which irritate the nasal passages and/or pharynx according to the invention, are any substances which, when administered abusively via the nasal passages and/or pharynx, bring about a physical reaction which is either so unpleasant for the abuser that he/she does not wish to or cannot continue administration, for example burning, or physiologically counteracts taking of the corresponding opioid, for example due to increased nasal secretion or sneezing. These substances which conventionally irritate the nasal passages and/or pharynx may also bring about a very unpleasant sensation or even unbearable pain when administered parenterally, in particular intravenously, such that the abuser does not wish to or cannot continue taking the substance. Particularly suitable substances which irritate the nasal passages and/or pharynx are those which cause burning, itching, urge to sneeze, increased formation of secretions or a combination of at least two of these stimuli. Appropriate substances and the quantities thereof which are conventionally to be used are known per se to the person skilled in the art or may be identified by simple preliminary testing.

Component (a) is preferably based on one or more constituents or one or more plant parts of at least one hot substance drug. Corresponding hot substance drugs are known per se to the person skilled in the art and are described, for example, in “Pharmazeutische Biologie—Drogen and ihre Inhaltsstoffe” by Prof. Dr. Hildebert Wagner, 2nd. revised edition, Gustav Fischer Verlag, Stuttgart-New York, 1982, pages 82 et seq.

The dosage form obtained by the process according to the invention may preferably contain the plant parts of the corresponding hot substance drugs in a quantity of 0.01 to 30 wt. %, particularly preferably of 0.1 to 0.5 wt. %, in each case relative to the total weight of the dosage form. If one or more constituents of corresponding hot substance drugs are used, the quantity thereof in a dosage unit obtained by the process according to the invention preferably amounts to 0.001 to 0.005 wt. %, relative to the total weight of the dosage form.

One or more constituents of at least one hot substance drug selected from the group comprising Allii sativi bulbus (garlic), Asari rhizoma cum herba (Asarum root and leaves), Calami rhizoma (calamus root), Capsici fructus (capsicum), Capsici fructus acer (cayenne pepper), Curcumae longae rhizoma (turmeric root), Curcumae xanthorrhizae rhizoma (Javanese turmeric root), Galangae rhizoma (galangal root), Myristicae semen (nutmeg), Piperis nigri fructus (pepper), Sinapis albae semen (white mustard seed), Sinapis nigri semen (black mustard seed), Zedoariae rhizoma (zedoary root) and Zingiberis rhizoma (ginger root), particularly preferably from the group comprising Capsici fructus (capsicum), Capsici fructus acer (cayenne pepper) and Piperis nigri fructus (pepper) may preferably be contained as component (a) to the dosage form according to the invention.

The constituents of the hot substance drugs preferably comprise o-methoxy(methyl)phenol compounds, acid amide compounds, mustard oils or sulfide compounds or compounds derived therefrom. Particularly preferably, at least one constituent of the hot substance drugs is selected from the group consisting of myristicin, elemicin, isoeugenol, α-asarone, safrole, gingerols, xanthorrhizol, capsaicinoids, preferably capsaicin, capsaicin derivatives, such as N-vanillyl-9E-octadecenamide, dihydrocapsaicin, nordihydrocapsaicin, homocapsaicin, norcapsaicin and nomorcapsaicin, piperine, preferably trans-piperine, glucosinolates, preferably based on non-volatile mustard oils, particularly preferably based on p-hydroxybenzyl mustard oil, methylmercapto mustard oil or methylsulfonyl mustard oil, and compounds derived from these constituents.

In another preferred embodiment, the dosage form according to the invention comprises component (b), i.e. a viscosity-increasing agent and/or gelling agent, which, with the assistance of a necessary minimum quantity of an aqueous liquid, forms a gel with the extract obtained from the dosage form, which gel is virtually impossible to administer safely, and preferably remains visually distinguishable when introduced into a further quantity of an aqueous liquid.

For the purposes of the specification, visually distinguishable means that the opioid-containing gel formed with the assistance of a necessary minimum quantity of aqueous liquid, when introduced, preferably with the assistance of a hypodermic needle, into a further quantity of aqueous liquid at 37° C., remains substantially insoluble and cohesive and cannot straightforwardly be dispersed in such a manner that it can safely be administered parenterally, in particular intravenously. The material preferably remains visually distinguishable for at least one minute, preferably for at least 10 minutes.

The increased viscosity of the extract makes it more difficult or even impossible for it to be passed through a needle or injected. If the gel remains visually distinguishable, this means that the gel obtained on introduction into a further quantity of aqueous liquid, for example by injection into blood, initially remains in the form of a largely cohesive thread, which, while it may indeed be broken up mechanically into smaller fragments, cannot be dispersed or even dissolved in such a manner that it can safely be administered parenterally, in particular intravenously. Intravenous administration of such a gel would therefore most probably result in serious damage to the health of the abuser. In combination with at least one optionally present component (a) or (c) to (d), this additionally leads to unpleasant burning, vomiting, bad flavor and/or visual deterrence.

In order to verify whether a viscosity-increasing agent and/or gelling agent is suitable as component (b) in the dosage form according to the invention, the opioid is preferably mixed with the viscosity-increasing agent and suspended in 10 ml of water at a temperature of 25° C. If this results in the formation of a gel which fulfils the above-stated conditions, the corresponding viscosity-increasing agent is suitable for preventing or averting abuse of the dosage forms according to the invention.

Preferred viscosity-increasing agents and/or gelling agents include but are not limited to the group consisting of microcrystalline cellulose, e.g. with 11 wt. % carboxymethylcellulose sodium (Avicel® RC 591), carboxymethylcellulose sodium (Blanose®, CMC-Na C300P®, Frimulsion® BLC-5, Tylose® C300 P), locust bean flour (Cesagum® LA-200, Cesagum® LID/150, Cesagum® LN-1), pectins such as citrus pectin (Cesapectin® HM Medium Rapid Set), apple pectin, pectin from lemon peel, waxy maize starch (C*Gel® 04201), sodium alginate (Frimulsion® ALG (E401)), guar flour (Frimulsion® BM, Polygum® 26/1-75), iota carrageenan (Frimulsion® D021), karaya gum, gellan gum (Kelcogel® F, Kelcogel® LT100), galactomannan (Meyprogat® 150), tara stone flour (Polygum® 43/1), propylene glycol alginate (Protanal®-Ester SD-LB), sodium hyaluronate, tragacanth, tara gum (Vidogum® SP 200), fermented polysaccharide welan gum (K1A96), xanthan gum (Xantural® 180). The names stated in brackets are the trade names by which exemplified materials are known commercially. In general, a quantity of 0.1 to 5 wt. % of the viscosity-increasing agent(s) is sufficient to fulfill the above-stated conditions. Component (b), where provided, is preferably present in the dosage form according to the invention in quantities of 5 mg per dosage form.

In a particularly preferred embodiment, the viscosity-increasing agents and/or gelling agents that are present as component (b) are those which, on extraction from the dosage form with the necessary minimum quantity of aqueous liquid, form a gel which encloses air bubbles. The resultant gels are distinguished by a turbid appearance, which provides the potential abuser with an additional optical warning and discourages him/her from administering the gel parenterally.

It is also possible to formulate the viscosity-increasing agent and the other constituents in the dosage form according to the invention in a mutually spatially separated arrangement.

In still another preferred embodiment, the dosage form according to the invention comprises component (c), i.e. an emetic, which is preferably present in a spatially separated arrangement from the other components of the dosage form according to the invention and, when correctly used, is intended not to exert its effect in the body.

Suitable emetics for preventing abuse of an opioid are known to the person skilled in the art and may be present in the dosage form according to the invention as such or in the form of corresponding derivatives, in particular esters or ethers, or in each case in the form of corresponding physiologically acceptable compounds, in particular in the form of the salts or solvates thereof. An emetic based on one or more constituents of ipecacuanha (ipecac) root, preferably based on the constituent emetine may preferably be considered in the dosage form according to the invention, as are, for example, described in “Pharmazeutische Biologie—Drogen and ihre Inhaltsstoffe” by Prof. Dr. Hildebert Wagner, 2nd, revised edition, Gustav Fischer Verlag, Stuttgart, New York, 1982.

The dosage form according to the invention may preferably comprise the emetic emetine as component (c), preferably in a quantity of ≧10 mg, particularly preferably of ≧20 mg and very particularly preferably in a quantity of ≧40 mg per dosage form. Apomorphine may likewise preferably be used as an emetic for additional abuse-proofing, preferably in a quantity of preferably ≧3 mg, particularly preferably of ≧5 mg and very particularly preferably of ≧7 mg per administration unit.

In yet another preferred embodiment, the dosage form according to the invention comprises component (d), i.e. a dye, which brings about an intense coloration of a corresponding aqueous solution, in particular when the attempt is made to extract the opioid for parenteral, preferably intravenous administration, which coloration may act as a deterrent to the potential abuser. Suitable dyes and the quantities required for the necessary deterrence may be found e.g. in WO 03/015531.

In another preferred embodiment, the dosage form according to the invention comprises component (e), i.e. a bittering agent. The consequent impairment of the flavor of the dosage form additionally prevents oral and/or nasal abuse. Suitable bitter substances and the quantities effective for use may be found in US-2003/0064099 A1. Suitable bitter substances are preferably aromatic oils, preferably peppermint oil, eucalyptus oil, bitter almond oil, menthol, fruit aroma substances, preferably aroma substances from lemons, oranges, limes, grapefruit or mixtures thereof, and/or denatonium benzoate.

Preferred components (f), i.e. surfactants according to the invention, are nonionic, anionic or cationic surfactants. Ionic surfactants are particularly preferred. It has been found that surfactants can function as aversive agents when the opioid agonist is abused via a mucosa, e.g. nasally, resulting in an unpleasant burning sensation.

In a preferred embodiment, the surfactant has a HLB value (hydrophilic-lipophilic-balance) within the range of 10±9, more preferably 10±6, most preferably 10±3; or 15±9, more preferably 15±6, most preferably 15±3; or 20±9, more preferably 20±6, most preferably 20±3; or 25±9, more preferably 25±6, most preferably 25±3; or 30±9, more preferably 30±6, most preferably 30±3; or 35±9, more preferably 35±6, most preferably 35±3.

A preferred example of an anionic surfactant is sodium laurylsulfate.

Particularly when components (c) and/or (e) are contained in the dosage form according to the invention, care should taken to ensure that they are formulated in such a manner or are present in such a low dose that, when correctly administered, the dosage form is able to bring about virtually no aversive effect which impairs the patient or the efficacy of the opioid. If the dosage form according to the invention contains component (c) and/or (e), the dosage must be selected such that, when correctly orally administered, no negative effect is caused. If, however, the intended dosage of the dosage form is exceeded inadvertently, in particular by children, or in the event of abuse, nausea or an inclination to vomit or a bad flavor are produced. The particular quantity of component (c) and/or (e) which can still be tolerated by the patient in the event of correct oral administration may be determined by the person skilled in the art by simple preliminary testing.

If, however, irrespective of the fact that the dosage form according to the invention is virtually impossible to pulverize, the dosage form containing the components (c) and/or (e) is provided with protection, these components should preferably be used at a dosage which is sufficiently high that, when abusively administered, they bring about an intense aversive effect on the abuser.

This is preferably achieved by spatial separation of at least the opioid from components (c) and/or (e), wherein the opioid is present in at least one subunit (X) and components (c) and/or (e) is/are present in at least one subunit (Y), and wherein, when the dosage form is correctly administered, components (c) and (e) do not exert their effect on taking and/or in the body and the remaining components of the formulation are identical.

If the dosage form according to the invention comprises at least 2 of components (c) or (e), these may each be present in the same or different subunits (Y). Preferably, when present, all the components (c) and (e) are present in one and the same subunit (Y). For the purposes of the specification, subunits are solid formulations, which in each case, apart from conventional auxiliary substances known to the person skilled in the art, contain the opioid, preferably also at least the polyalkylene oxide and optionally at least one of the optionally present components (a) and/or (b) and/or (c) and/or (d) and/or (e) and/or (f).

One substantial advantage of the separated formulation of opioids from components (c) or (e) in subunits (X) and (Y) of the dosage form according to the invention is that, when correctly administered, components (c) and/or (e) are hardly released in the body or are released in such small quantities that they exert no effect which impairs the patient or therapeutic success or, on passing through the patient's body, they are only liberated in locations where they cannot be sufficiently absorbed to be effective. When the dosage form is correctly administered, preferably hardly any of components (c) and/or (e) is released into the patient's body or they go unnoticed by the patient. The person skilled in the art will understand that the above-stated conditions may vary as a function of the particular components (c) and/or (e) and of the formulation of the subunits or the dosage form. The optimum formulation for the particular dosage form may be determined by simple preliminary testing.

Should, contrary to expectations, the abuser succeed in comminuting such a dosage form according to the invention, which comprises components (c) and/or (d) and/or (e) and/or (f) in subunits (Y), for the purpose of abusing the opioid and obtain a powder which is extracted with a suitable extracting agent, not only the opioid but also the particular component (c) and/or (d) and/or (e) and/or (f) will be obtained in a form in which it cannot readily be separated from the opioid, such that when the dosage form which has been tampered with is administered, in particular by oral and/or parenteral administration, it will exert its effect on taking and/or in the body combined with an additional aversive effect on the abuser corresponding to component (c) and/or (e) or, when the attempt is made to extract the opioid, the coloration caused by component (d) will act as a deterrent and so prevent abuse of the dosage form.

A dosage form in which the opioid is spatially separated from components (c) and/or (d), preferably by formulation in different subunits, may be formulated according to the invention in many different ways, wherein the corresponding subunits of such a dosage form may each be present in any desired spatial arrangement relative to one another, provided that the above-stated conditions for the release of components (c) and/or (d) are fulfilled.

The person skilled in the art will understand that component(s) (a) and/or (b) and/or (f) which are optionally also present may preferably be formulated in the dosage form according to the invention both in the particular subunits (X) and (Y) and in the form of independent subunits corresponding to subunits (X) and (Y), provided that neither the abuse-proofing nor the opioid release in the event of correct administration is impaired by the nature of the formulation.

In a preferred embodiment of the dosage form according to the invention, subunits (X) and (Y) are present in multiparticulate form, wherein granules, spheroids, beads or pellets are preferred and the same form, i.e. shape, is selected for both subunit (X) and subunit (Y), such that it is not possible to separate subunits (X) from (Y) by mechanical selection. The multiparticulate forms are preferably of a size in the range from 0.1 to 3 mm, preferably of 0.5 to 2 mm. The subunits (X) and (Y) in multiparticulate form may also preferably be press-moulded into a tablet, wherein the final formulation in each case proceeds in such a manner that the subunits (X) and (Y) are also retained in the resultant dosage form. The multiparticulate subunits (X) and (Y) of identical shape should also not be visually distinguishable from one another so that the abuser cannot separate them from one another by simple sorting. This may, for example, be achieved by the application of identical coatings which, apart from this disguising function, may also incorporate further functions, such as, for example, delayed release of one or more opioids or provision of a finish resistant to gastric juices on the particular subunits.

In a further preferred embodiment of the present invention, subunits (X) and (Y) are in each case arranged in layers relative to one another. The layered subunits (X) and (Y) are preferably arranged for this purpose vertically or horizontally relative to one another in the dosage form according to the invention, wherein in each case one or more layered subunits (X) and one or more layered subunits (Y) may be present in the dosage form, such that, apart from the preferred layer sequences (X)-(Y) or (X)-(Y)-(X), any desired other layer sequences may be considered, optionally in combination with layers containing components (a) and/or (b).

Another preferred dosage form according to the invention is one in which subunit (Y) forms a core which is completely enclosed by subunit (X), wherein a separation layer (Z) may be present between said layers. Such a structure is preferably also suitable for the above-stated multiparticulate forms, wherein both subunits (X) and (Y) and an optionally present separation layer (Z), which should preferably satisfy the hardness requirement according to the invention, are then formulated in one and the same multiparticulate form using the process according to the invention.

In a further preferred embodiment of the dosage form according to the invention, the subunit (X) forms a core, which is enclosed by subunit (Y), wherein the latter comprises at least one channel which leads from the core to the surface of the dosage form.

The dosage form according to the invention may comprise, between one layer of the subunit (X) and one layer of the subunit (Y), in each case one or more, preferably one, optionally swellable separation layer (Z) which serves to separate subunit (X) spatially from (Y).

If the dosage form according to the invention comprises the layered subunits (X) and (Y) and an optionally present separation layer (Z) in an at least partially vertical or horizontal arrangement, the dosage form preferably takes the form of a tablet, a coextrudate or a laminate, which has been produced using the process according to the invention.

In one particularly preferred embodiment, the entirety of the free surface of subunit (Y) and optionally at least part of the free surface of subunit(s) (X) and optionally at least part of the free surface of the optionally present separation layer(s) (Z) may be coated with at least one barrier layer (Z′) which prevents release of component (c) and/or (d) and/or (c) and/or (e) and/or (f). The barrier layer (Z′) should preferably also fulfill the hardness conditions according to the invention.

Another particularly preferred embodiment of the dosage form according to the invention comprises a vertical or horizontal arrangement of the layers of subunits (X) and (Y) and at least one push layer (p) arranged there between, and optionally a separation layer (Z), in which dosage form the entirety of the free surface of the layer structure consisting of subunits (X) and (Y), the push layer and the optionally present separation layer (Z) is provided with a semipermeable coating (E), which is permeable to a release medium, i.e. conventionally a physiological liquid, but substantially impermeable to the opioid and to component (c) and/or (e), and wherein this coating (E) comprises at least one opening for release of the opioid in the area of subunit (X).

In a further preferred embodiment, the subunit (X) of the dosage form according to the invention is in the form of a tablet, the edge face and optionally one of the two main faces of which is covered with a barrier layer (Z′) containing component (c) and/or (e).

The person skilled in the art will understand that the auxiliary substances of the subunit(s) (X) or (Y) and of the optionally present separation layer(s) (Z) and/or of the barrier layer(s) (Z′) used in the production according to the invention of the respective dosage form will vary as a function of the arrangement thereof in the dosage form, the mode of administration and as a function of the particular opioid of the optionally present components (a) and/or (b) and/or (d) and of component (c) and/or (e). The materials which have the requisite properties are in each case known per se to the person skilled in the art.

If release of component (c) and/or (e) from subunit (Y) of the dosage form according to the invention is prevented with the assistance of a cover, preferably a barrier layer, the subunit may consist of conventional materials known to the person skilled in the art, preferably contain the polyalkylene oxide and preferably be produced according to the invention.

If a corresponding barrier layer (Z′) is not provided to prevent release of component (c) and/or (e), the materials of the subunits should be selected such that release of the particular component (c) from subunit (Y) is virtually ruled out.

The materials which are stated below to be suitable for production of the barrier layer may preferably be used for this purpose and should preferably contain the polyalkylene oxide for fulfilling the hardness conditions.

Preferred materials are those which are selected from the group consisting of alkylcelluloses, hydroxyalkylcelluloses, glucans, scleroglucans, mannans, xanthans, copolymers of poly[bis(p-carboxyphenoxy)propane:sebacic acid], preferably in a molar ratio of 20:80 (marketed under the name Polifeprosan 20®), carboxymethylcelluloses, cellulose ethers, cellulose esters, nitrocelluloses, polymers based on (meth)acrylic acid and the esters thereof, polyamides, polycarbonates, polyalkylenes, polyalkylene glycols, polyalkylene oxides, polyalkylene terephthalates, polyvinyl alcohols, polyvinyl ethers, polyvinyl esters, halogenated polyvinyls, polyglycolides, polysiloxanes and polyurethanes and the copolymers thereof. Particularly suitable materials may be selected from the group consisting of methylcellulose, ethylcellulose, hydroxypropylcellulose, hydroxypropylmethylcellulose, hydroxybutylmethylcellulose, cellulose acetate, cellulose propionate (of low, medium or high molecular weight), cellulose acetate propionate, cellulose acetate butyrate, cellulose acetate phthalate, carboxymethylcellulose, cellulose triacetate, sodium cellulose sulfate, polymethyl methacrylate, polyethyl methacrylate, polybutyl methacrylate, polyisobutyl methacrylate, polyhexyl methacrylate, polyisodecyl methacrylate, polylauryl methacrylate, polyphenyl methacrylate, polymethyl acrylate, polyisopropyl acrylate, polyisobutyl acrylate, polyoctadecyl acrylate, polyethylene, low density polyethylene, high density polyethylene, polypropylene, polyethylene glycol, polyethylene oxide, polyethylene terephthalate, polyvinyl alcohol, polyvinyl isobutyl ether, polyvinyl acetate and polyvinyl chloride.

Particularly suitable copolymers may be selected from the group comprising copolymers of butyl methacrylate and isobutyl methacrylate, copolymers of methyl vinyl ether and maleic acid of high molecular weight, copolymers of methyl vinyl ether and maleic acid monoethyl ester, copolymers of methyl vinyl ether and maleic anhydride and copolymers of vinyl alcohol and vinyl acetate. Further materials which are particularly suitable for formulating the barrier layer are starch-filled polycaprolactone, aliphatic polyesteramides, aliphatic and aromatic polyester urethanes, polyhydroxyalkanoates, in particular polyhydroxybutyrates, polyhydroxyvalerates, casein, polylactides and copolylactides.

The above-stated materials may optionally be blended with further conventional auxiliary substances known to the person skilled in the art, preferably selected from the group consisting of glyceryl monostearate, semi-synthetic triglyceride derivatives, semi-synthetic glycerides, hydrogenated castor oil, glyceryl palmitostearate, glyceryl behenate, polyvinyl-pyrrolidone, gelatine, magnesium stearate, stearic acid, sodium stearate, talcum, sodium benzoate, boric acid and colloidal silica, fatty acids, substituted triglycerides, glycerides, polyoxyalkylene glycols and the derivatives thereof.

If the dosage form according to the invention comprises a separation layer (Z′), said layer, like the uncovered subunit (Y), may preferably consist of the above-stated materials described for the barrier layer. The person skilled in the art will understand that release of the opioid or of the aversive agent from the particular subunit may be controlled by the thickness of the separation layer.

Besides the opioid agonist, the opioid antagonist and the polyalkylene oxide the pharmaceutical dosage form according to the invention may contain further constituents, such as conventional pharmaceutical excipients.

Preferably, the pharmaceutical dosage form according to the invention contains a plasticizer.

The plasticizer improves the processability of the polyalkylene oxide. A preferred plasticizer is polyalkylene glycol, like polyethylene glycol, triacetin, fatty acids, fatty acid esters, waxes and/or microcrystalline waxes. Particularly preferred plasticizers are polyethylene glycols, such as PEG 6000.

Preferably, the content of the plasticizer is within the range of from 0.1 to 30 wt.-% or 0.1 to 25 wt.-% more preferably 0.5 to 22.5 wt.-%, still more preferably 1.0 to 20 wt.-%, yet more preferably 2.5 to 17.5 wt.-%, most preferably 5.0 to 15 wt.-% and in particular 7.5 to 12.5 wt.-%, based on the total weight of the pharmaceutical dosage form.

In a preferred embodiment, the plasticizer is a polyalkylene glycol having a content within the range of 1.0±0.7 wt.-%, more preferably 1.0±0.6 wt.-%, still more preferably 1.0±0.5 wt.-%, yet more preferably 1.0±0.4 wt.-%, most preferably 1.0±0.3 wt.-%, and in particular 1.0±0.2 wt.-%, based on the total weight of the pharmaceutical dosage form.

In another preferred embodiment, the plasticizer is a polyalkylene glycol having a content within the range of 5±4 wt.-%, more preferably 5±3.5 wt.-%, still more preferably 5±3 wt.-%, yet more preferably 5±2.5 wt.-%, most preferably 5±2 wt.-%, and in particular 5±1.5 wt.-%, based on the total weight of the pharmaceutical dosage form.

In still another preferred embodiment, the plasticizer is a polyalkylene glycol having a content within the range of 10±8 wt.-%, more preferably 10±6 wt.-%, still more preferably 10±5 wt.-%, yet more preferably 10±4 wt.-%, most preferably 10±3 wt.-%, and in particular 10±2 wt.-%, based on the total weight of the pharmaceutical dosage form.

In yet another preferred embodiment, the plasticizer is a polyalkylene glycol having a content within the range of 15±8 wt.-%, more preferably 15±6 wt.-%, still more preferably 15±5 wt.-%, yet more preferably 15±4 wt.-%, most preferably 15±3 wt.-%, and in particular 15±2 wt.-%, based on the total weight of the pharmaceutical dosage form.

In a further preferred embodiment, the plasticizer is a polyalkylene glycol having a content within the range of 20±8 wt.-%, more preferably 20±6 wt.-%, still more preferably 20±5 wt.-%, yet more preferably 20±4 wt.-%, most preferably 20±3 wt.-%, and in particular 20±2 wt.-%, based on the total weight of the pharmaceutical dosage form.

In still a further preferred embodiment, the plasticizer is a polyalkylene glycol having a content within the range of 25±8 wt.-%, more preferably 25±6 wt.-%, still more preferably 25±5 wt.-%, yet more preferably 25±4 wt.-%, most preferably 25±3 wt.-%, and in particular 25±2 wt.-%, based on the total weight of the pharmaceutical dosage form.

Preferably, the pharmaceutical dosage form according to the invention contains an antioxidant.

Suitable antioxidants include ascorbic acid, α-tocopherol (vitamin E), butylhydroxyanisol, butylhydroxytoluene, salts of ascorbic acid (vitamin C), ascorbylic palmitate, monothioglycerine, coniferyl benzoate, nordihydroguajaretic acid, gallus acid esters, phosphoric acid, and the derivatives thereof, such as vitamin E-succinate or vitamin E-palmitate and/or sodium bisulphite, more preferably butylhydroxytoluene (BHT) or butylhydroxyanisol (BHA) and/or α-tocopherol.

Preferably, the content of the antioxidant is within the range of from 0.001 to 5.0 wt.-%, more preferably 0.002 to 2.5 wt.-%, more preferably 0.003 to 1.5 wt.-%, still more preferably 0.005 to 1.0 wt.-%, yet more preferably 0.01 to 0.5 wt.-%, most preferably 0.05 to 0.4 wt.-% and in particular 0.05 to 0.15 wt.-% or 0.1 to 0.3 wt.-%, based on the total weight of the pharmaceutical dosage form.

A particularly preferred antioxidant is α-tocopherol.

In a preferred embodiment, the content of α-tocopherol is within the range of 0.1±0.08 wt.-%, more preferably 0.1±0.07 wt.-%, still more preferably 0.1±0.06 wt.-%, yet more preferably 0.1±0.05 wt.-%, most preferably 0.1±0.04 wt.-%, and in particular 0.1±0.03 wt.-%, based on the total weight of the pharmaceutical dosage form.

In another preferred embodiment, the content of α-tocopherol is within the range of 0.2±0.18 wt.-%, more preferably 0.2±0.15 wt.-%, still more preferably 0.2±0.12 wt.-%, yet more preferably 0.2±0.09 wt.-%, most preferably 0.2±0.06 wt.-%, and in particular 0.2±0.03 wt.-%, based on the total weight of the pharmaceutical dosage form.

In a preferred embodiment, when the pharmaceutical dosage form additionally comprises an acid, the relative weight ratio of the acid, preferably citric acid, and the antioxidant, preferably α-tocopherol, is within the range of from 10:1 to 1:10, more preferably 8:1 to 1:8 or 9:1 to 1:5, still more preferably 6:1 to 1:6 or 8:1 to 1:3, yet more preferably 5:1 to 1:4 or 7:1 to 1:1, most preferably 4:1 to 1:3 or 6:1 to 3:1 and in particular 3:1 to 1:2, 2:1 to 1:2 or 6:1 to 4:1.

The pharmaceutical dosage form according to the invention preferably contains a free physiologically acceptable acid in an amount of from 0.001 to 5.0 wt.-%, based on the total weight of the pharmaceutical dosage form. The acid may be organic or inorganic, liquid or solid. Solid acids are preferred, particularly crystalline organic or inorganic acids.

Preferably, the acid is free. This means that the acidic functional groups of the acid are not all together constituents of a salt of the opioid agonist and the opioid antagonist, respectively. If the opioid agonist and/or the opioid antagonist is present as a salt of an acid, e.g. as hydrochloride, the pharmaceutical dosage form according to the invention preferably contains as acid another, chemically different acid which is not present as a constituent of the salt of the opioid agonist and the opioid antagonist, respectively. In other words, monoacids that form a salt with opioid agonist or opioid antagonist are not to be considered as free acids in the meaning of the present invention. When acid has more than a single acidic functional group (e.g. phosphoric acid), the acid may be present as a constituent of a salt of the opioid agonist or the opioid antagonist, provided that at least one of the acidic functional groups of the acid is not involved in the formation of the salt, i.e. is free. Preferably, however, each and every acidic functional group of acid is not involved in the formation of a salt with opioid agonist and opioid antagonist. It is also possible, however, that free acid and the acid forming a salt with opioid agonist or opioid antagonist are identical. Under these circumstances the acid is preferably present in molar excess compared to opioid agonist and opioid antagonist, respectively.

In a preferred embodiment, the acid contains at least one acidic functional group (e.g. —CO₂H, —SO₃H, —PO₃H₂, —OH and the like) having a pK_{A} value within the range of 2.00±1.50, more preferably 2.00±1.25, still more preferably 2.00±1.00, yet more preferably 2.00±0.75, most preferably 2.00±0.50 and in particular 2.00±0.25. In another preferred embodiment, the acid contains at least one acidic functional group having a pK_{A} value within the range of 2.25±1.50, more preferably 2.25±1.25, still more preferably 2.25±1.00, yet more preferably 2.25±0.75, most preferably 2.25±0.50 and in particular 2.25±0.25. In another preferred embodiment, the acid contains at least one acidic functional group having a pK_{A} value within the range of 2.50±1.50, more preferably 2.50±1.25, still more preferably 2.50±1.00, yet more preferably 2.50±0.75, most preferably 2.50±0.50 and in particular 2.50±0.25. In another preferred embodiment, the acid contains at least one acidic functional group having a pK_{A} value within the range of 2.75±1.50, more preferably 2.75±1.25, still more preferably 2.75±1.00, yet more preferably 2.75±0.75, most preferably 2.75±0.50 and in particular 2.75±0.25. In another preferred embodiment, the acid contains at least one acidic functional group having a pK_{A} value within the range of 3.00±1.50, more preferably 3.00±1.25, still more preferably 3.00±1.00, yet more preferably 3.00±0.75, most preferably 3.00±0.50 and in particular 3.00±0.25. In still another preferred embodiment, the acid contains at least one acidic functional group having a pK_{A} value within the range of 3.25±1.50, more preferably 3.25±1.25, still more preferably 3.25±1.00, yet more preferably 3.25±0.75, most preferably 3.25±0.50 and in particular 3.25±0.25.

In yet another preferred embodiment, the acid contains at least one acidic functional group having a pK_{A} value within the range of 4.50±1.50, more preferably 4.50±1.25, still more preferably 4.50±1.00, yet more preferably 4.50±0.75, most preferably 4.50±0.50 and in particular 4.50±0.25. In yet another preferred embodiment, the acid contains at least one acidic functional group having a pK_{A} value within the range of 4.75±1.50, more preferably 4.75±1.25, still more preferably 4.75±1.00, yet more preferably 4.75±0.75, most preferably 4.75±0.50 and in particular 4.75±0.25. In yet another preferred embodiment, the acid contains at least one acidic functional group having a pK_{A} value within the range of 5.00±1.50, more preferably 5.00±1.25, still more preferably 5.00±1.00, yet more preferably 5.00±0.75, most preferably 5.00±0.50 and in particular 5.00±0.25.

Preferably, the acid is an organic carboxylic or sulfonic acid, particularly a carboxylic acid. Multicarboxylic acids and/or hydroxy-carboxylic acids are especially preferred.

In case of multicarboxylic acids, the partial salts thereof are also to be regarded as multi-carboxylic acids, e.g. the partial sodium, potassium or ammonium salts. For example, citric acid is a multicarboxylic acid having three carboxyl groups. As long as there remains at least one carboxyl group protonated (e.g. sodium dihydrogen citrate or disodium hydrogen citrate), the salt is to be regarded as a multicarboxylic acid. Preferably, however, all carboxyl groups of the multicarboxylic acid are protonated.

Preferably, the acid is of low molecular weight, i.e., not polymerized. Typically, the molecular weight of the acid is below 500 g/mol.

Examples of acids include saturated and unsaturated monocarboxylic acids, saturated and unsaturated bicarboxylic acids, tricarboxylic acids, α-hydroxyacids and β-hydroxylacids of monocarboxylic acids, α-hydroxyacids and β-hydroxyacids of bicarboxylic acids, α-hydroxyacids and β-hydroxyacids of tricarboxylic acids, ketoacids, α-ketoacids, β-ketoacids, of the polycarboxylic acids, of the polyhydroxy monocarboxylic acids, of the polyhydroxy bicarboxylic acids, of the polyhydroxy tricarboxylic acids.

Preferably, the acid is selected from the group consisting of benzenesulfonic acid, citric acid, α-glucoheptonic acid, D-gluconic acid, glycolic acid, lactic acid, malic acid, malonic acid, mandelic acid, propanoic acid, succinic acid, tartaric acid (d, l, or dl), tosic acid (toluene-sulfonic acid), valeric acid, palmitic acid, pamoic acid, sebacic acid, stearic acid, lauric acid, acetic acid, adipic acid, glutaric acid, 4-chlorobenzenesulfonic acid, ethanedisulfonic acid, ethylsuccinic acid, fumaric acid, galactaric acid (mucic acid), D-glucuronic acid, 2-oxo-glutaric acid, glycerophosphoric acid, hippuric acid, isethionic acid (ethanolsulfonic acid), lactobionic acid, maleic acid, maleinic acid, 1,5-naphthalene-disulfonic acid, 2-naphthalene-sulfonic acid, pivalic acid, terephthalic acid, thiocyanic acid, cholic acid, n-dodecyl sulfate, 3-hydroxy-2-naphthoic acid, 1-hydroxy-2-naphthoic acid, oleic acid, undecylenic acid, ascorbic acid, (+)-camphoric acid, d-camphorsulfonic acid, dichloroacetic acid, ethanesulfonic acid, formic acid, methanesulfonic acid, nicotinic acid, orotic acid, oxalic acid, picric acid, L-pyroglutamic acid, saccharine, salicylic acid, gentisic acid, and/or 4-acetamidobenzoic acid.

The content of the acid is preferably within the range of from 0.001 to 5.0 wt.-%, preferably 0.005 to 2.5 wt.-%, more preferably 0.01 to 2.0 wt.-%, still more preferably 0.05 to 1.5 wt.-%, most preferably 0.1 to 1.0 wt.-% and in particular 0.2 to 0.9 wt.-%, based on the total weight of the pharmaceutical dosage form.

Preferably, the acid is a multicarboxylic acid. More preferably, the multicarboxylic acid is selected from the group consisting of citric acid, maleic acid and fumaric acid.

Citric acid is particularly preferred.

The multicarboxylic acid, preferably citric acid, may be present in its anhydrous form or as a solvate and hydrate, respectively, e.g., as monohydrate.

In a preferred embodiment, the content of the acid, preferably citric acid, is within the range of 0.1±0.08 wt.-%, more preferably 0.1±0.07 wt.-%, still more preferably 0.1±0.06 wt.-%, yet more preferably 0.1±0.05 wt.-%, most preferably 0.1±0.04 wt.-%, and in particular 0.1±0.03 wt.-%, based on the total weight of the pharmaceutical dosage form.

In another preferred embodiment, the content of the acid, preferably citric acid, is within the range of 0.2±0.18 wt.-%, more preferably 0.2±0.15 wt.-%, still more preferably 0.2±0.12 wt.-%, yet more preferably 0.2±0.09 wt.-%, most preferably 0.2±0.06 wt.-%, and in particular 0.2±0.03 wt.-%, based on the total weight of the pharmaceutical dosage form.

In still another preferred embodiment, the content of the acid, preferably citric acid, is within the range of 0.3±0.18 wt.-%, more preferably 0.3±0.15 wt.-%, still more preferably 0.3±0.12 wt.-%, yet more preferably 0.3±0.09 wt.-%, most preferably 0.3±0.06 wt.-%, and in particular 0.3±0.03 wt.-%, based on the total weight of the pharmaceutical dosage form.

In yet another preferred embodiment, the content of the acid, preferably citric acid, is within the range of 0.4±0.18 wt.-%, more preferably 0.4±0.15 wt.-%, still more preferably 0.4±0.12 wt.-%, yet more preferably 0.4±0.09 wt.-%, most preferably 0.4±0.06 wt.-%, and in particular 0.4±0.03 wt.-%, based on the total weight of the pharmaceutical dosage form.

In a further preferred embodiment, the content of the acid, preferably citric acid, is within the range of 0.5±0.18 wt.-%, more preferably 0.5±0.15 wt.-%, still more preferably 0.5±0.12 wt.-%, yet more preferably 0.5±0.09 wt.-%, most preferably 0.5±0.06 wt.-%, and in particular 0.5±0.03 wt.-%, based on the total weight of the pharmaceutical dosage form.

In still a further preferred embodiment, the content of the acid, preferably citric acid, is within the range of 0.6±0.18 wt.-%, more preferably 0.6±0.15 wt.-%, still more preferably 0.6±0.12 wt.-%, yet more preferably 0.6±0.09 wt.-%, most preferably 0.6±0.06 wt.-%, and in particular 0.6±0.03 wt.-%, based on the total weight of the pharmaceutical dosage form.

In yet a further preferred embodiment, the content of the acid, preferably citric acid, is within the range of 0.7±0.18 wt.-%, more preferably 0.7±0.15 wt.-%, still more preferably 0.7±0.12 wt.-%, yet more preferably 0.7±0.09 wt.-%, most preferably 0.7±0.06 wt.-%, and in particular 0.7±0.03 wt.-%, based on the total weight of the pharmaceutical dosage form.

In still another preferred embodiment, the content of acid, preferably citric acid, is within the range of 0.8±0.18 wt.-%, more preferably 0.8±0.15 wt.-%, still more preferably 0.8±0.12 wt.-%, yet more preferably 0.8±0.09 wt.-%, most preferably 0.8±0.06 wt.-%, and in particular 0.8±0.03 wt.-%, based on the total weight of the pharmaceutical dosage form.

In yet another preferred embodiment, the content of the acid, preferably citric acid, is within the range of 0.85±0.18 wt.-%, more preferably 0.85±0.15 wt.-%, still more preferably 0.85±0.12 wt.-%, yet more preferably 0.85±0.09 wt.-%, most preferably 0.85±0.06 wt.-%, and in particular 0.85±0.03 wt.-%, based on the total weight of the pharmaceutical dosage form.

In a further preferred embodiment, the content of the acid, preferably citric acid, is within the range of 0.9±0.18 wt.-%, more preferably 0.9±0.15 wt.-%, still more preferably 0.9±0.12 wt.-%, yet more preferably 0.9±0.09 wt.-%, most preferably 0.9±0.06 wt.-%, and in particular 0.9±0.03 wt.-%, based on the total weight of the pharmaceutical dosage form.

In still a further preferred embodiment, the content of the acid, preferably citric acid, is within the range of 1.0±0.18 wt.-%, more preferably 1.0±0.15 wt.-%, still more preferably 1.0±0.12 wt.-%, yet more preferably 1.0±0.09 wt.-%, most preferably 1.0±0.06 wt.-%, and in particular 1.0±0.03 wt.-%, based on the total weight of the pharmaceutical dosage form.

The pharmaceutical dosage form according to the invention may also contain a natural, semi-synthetic or synthetic wax. Waxes with a softening point of at least 50° C., more preferably 60° C. are preferred. Carnauba wax and beeswax are particularly preferred, especially carnauba wax.

Preferably, the pharmaceutical dosage form according to the invention contains a coating, preferably a film-coating. Suitable coating materials are known to the skilled person. Suitable coating materials are commercially available, e.g. under the trademarks Opadry® and Eudragit®.

Examples of suitable materials include cellulose esters and cellulose ethers, such as methyl-cellulose (MC), hydroxypropylmethylcellulose (HPMC), hydroxypropylcellulose (HPC), hydroxyethylcellulose (HEC), sodium carboxymethylcellulose (Na-CMC), ethylcellulose (EC), cellulose acetate phthalate (CAP), hydroxypropylmethylcellulose phthalate (HPMCP); poly(meth)acrylates, such as aminoalkylmethacrylate copolymers, ethylacrylate methyl-methacrylate copolymers, methacrylic acid methylmethacrylate copolymers, methacrylic acid methylmethacrylate copolymers; vinyl polymers, such as polyvinylpyrrolidone, polyvinyl-acetatephthalate, polyvinyl alcohol, polyvinylacetate; and natural film formers, such as shellack.

In a particularly preferred embodiment, the coating is water-soluble. In a preferred embodiment, the coating is based on polyvinyl alcohol, such as polyvinyl alcohol-part. hydrolyzed, and may additionally contain polyethylene glycol, such as macrogol 3350, and/or pigments. In another preferred embodiment, the coating is based on hydroxypropylmethyl-cellulose, preferably hypromellose type 2910 having a viscosity of 3 to 15 mPas.

The coating of the pharmaceutical dosage form can increase its storage stability.

The coating can be resistant to gastric juices and dissolve as a function of the pH value of the release environment. By means of this coating, it is possible to ensure that the pharmaceutical dosage form according to the invention passes through the stomach undissolved and the active compound is only released in the intestines. The coating which is resistant to gastric juices preferably dissolves at a pH value of between 5 and 7.5. Corresponding materials and methods for the delayed release of active compounds and for the application of coatings which are resistant to gastric juices are known to the person skilled in the art, for example from “Coated Pharmaceutical dosage forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers.

The pharmaceutical dosage form according to the invention is preferably tamper-resistant. Preferably, tamper-resistance is achieved based on the mechanical properties of the pharmaceutical dosage form so that comminution is avoided or at least substantially impeded. According to the invention, the term comminution means the pulverization of the pharmaceutical dosage form using conventional means usually available to an abuser, for example a pestle and mortar, a hammer, a mallet or other conventional means for pulverizing under the action of force. Thus, tamper-resistance preferably means that pulverization of the pharmaceutical dosage form using conventional means is avoided or at least substantially impeded.

Preferably, the mechanical properties of the pharmaceutical dosage form according to the invention, particularly its breaking strength, substantially rely on the presence and spatial distribution of the polyalkylene oxide, although its mere presence does typically not suffice in order to achieve said properties. The advantageous mechanical properties of the pharmaceutical dosage form according to the invention may not automatically be achieved by simply processing opioid agonist, opioid antagonist, polyalkylene oxide, and optionally further excipients by means of conventional methods for the preparation of pharmaceutical dosage forms. In fact, usually suitable apparatuses must be selected for the preparation and critical processing parameters must be adjusted, particularly pressure/force, temperature and time. Thus, even if conventional apparatuses are used, the process protocols usually must be adapted in order to meet the required criteria.

Furthermore, tamper-resistance is achieved based on the poor solubility properties of the pharmaceutical dosage form in alcohol, especially ethanol, thereby effectively preventing alcohol dose dumping.

The pharmaceutical dosage form according to the invention has a breaking strength of at least 300 N, preferably at least 400 N, more preferably at least 500 N or at least 510 N or at least 520 N or at least 550 N, still more preferably at least 750 N, yet more preferably at least 1000 N, most preferably at least 1250 N and in particular at least 1500 N.

The “breaking strength” (resistance to crushing) of a pharmaceutical dosage form is known to the skilled person. In this regard it can be referred to, e.g., W. A. Ritschel, Die Tablette, 2. Auflage, Editio Cantor Verlag Aulendorf, 2002; H Liebermann et al., Pharmaceutical dosage forms: Tablets, Vol. 2, Informa Healthcare; 2 edition, 1990; and Encyclopedia of Pharmaceutical Technology, Informa Healthcare; 1 edition.

For the purpose of the specification, the breaking strength is preferably defined as the amount of force that is necessary in order to fracture the pharmaceutical dosage form (=breaking force). Therefore, for the purpose of the specification the pharmaceutical dosage form does preferably not exhibit the desired breaking strength when it breaks, i.e., is fractured into at least two independent parts that are separated from one another. In another preferred embodiment, however, the pharmaceutical dosage form is regarded as being broken if the force decreases by 25% (threshold value) of the highest force measured during the measurement (see below).

The pharmaceutical dosage forms according to the invention are distinguished from conventional pharmaceutical dosage forms in that, due to their breaking strength, they cannot be pulverized by the application of force with conventional means, such as for example a pestle and mortar, a hammer, a mallet or other usual means for pulverization, in particular devices developed for this purpose (tablet crushers). In this regard “pulverization” preferably means crumbling into small particles that would immediately release the pharmacologically active compound (A) in a suitable medium. Avoidance of pulverization virtually rules out oral or parenteral, in particular intravenous or nasal abuse.

Conventional tablets typically have a breaking strength well below 200 N in any direction of extension. The breaking strength of conventional round tablets may be estimated according to the following empirical formula: Breaking Strength [in N]=10×Diameter Of The Tablet [in mm]. Thus, according to said empirical formula, a round tablet having a breaking strength of at least 300 N would require a diameter of at least 30 mm). Such a tablet, however, could not be swallowed. The above empirical formula preferably does not apply to the pharmaceutical dosage forms of the invention, which are not conventional but rather special.

Further, the actual mean chewing force is about 220 N (cf., e.g., P. A. Proeschel et al., J Dent Res, 2002, 81(7), 464-468). This means that conventional tablets having a breaking strength well below 200 N may be crushed upon spontaneous chewing, whereas the pharmaceutical dosage forms according to the invention may not.

Still further, when applying a gravitational acceleration of about 9.81 m/s², 300 N correspond to a gravitational force of more than 30 kg, i.e. the pharmaceutical dosage forms according to the invention can preferably withstand a weight of more than 30 kg without being pulverized.

Methods for measuring the breaking strength of a pharmaceutical dosage form are known to the skilled artisan. Suitable devices are commercially available.

For example, the breaking strength (resistance to crushing) can be measured in accordance with the Eur. Ph. 5.0, 2.9.8 or 6.0, 2.09.08 “Resistance to Crushing of Tablets”. The test is intended to determine, under defined conditions, the resistance to crushing of tablets, measured by the force needed to disrupt them by crushing. The apparatus consists of 2 jaws facing each other, one of which moves towards the other. The flat surfaces of the jaws are perpendicular to the direction of movement. The crushing surfaces of the jaws are flat and larger than the zone of contact with the tablet. The apparatus is calibrated using a system with a precision of 1 Newton. The tablet is placed between the jaws, taking into account, where applicable, the shape, the break-mark and the inscription; for each measurement the tablet is oriented in the same way with respect to the direction of application of the force (and the direction of extension in which the breaking strength is to be measured). The measurement is carried out on 10 tablets, taking care that all fragments of tablets have been removed before each determination. The result is expressed as the mean, minimum and maximum values of the forces measured, all expressed in Newton.

A similar description of the breaking strength (breaking force) can be found in the USP. The breaking strength can alternatively be measured in accordance with the method described therein where it is stated that the breaking strength is the force required to cause a tablet to fail (i.e., break) in a specific plane. The tablets are generally placed between two plates, one of which moves to apply sufficient force to the tablet to cause fracture. For conventional, round (circular cross-section) tablets, loading occurs across their diameter (sometimes referred to as diametral loading), and fracture occurs in the plane. The breaking force of tablets is commonly called hardness in the pharmaceutical literature; however, the use of this term is misleading. In material science, the term hardness refers to the resistance of a surface to penetration or indentation by a small probe. The term crushing strength is also frequently used to describe the resistance of tablets to the application of a compressive load. Although this term describes the true nature of the test more accurately than does hardness, it implies that tablets are actually crushed during the test, which is often not the case.

Alternatively, the breaking strength (resistance to crushing) can be measured in accordance with WO 2005/016313, WO 2005/016314, and WO 2006/082099, which can be regarded as a modification of the method described in the Eur. Ph. The apparatus used for the measurement is preferably a “Zwick Z 2.5” materials tester, Fₘₐₓ=2.5 kN with a maximum draw of 1150 mm, which should be set up with one column and one spindle, a clearance behind of 100 mm and a test speed adjustable between 0.1 and 800 mm/min together with testControl software. Measurement is performed using a pressure piston with screw-in inserts and a cylinder (diameter 10 mm), a force transducer, Fₘₐₓ. 1 kN, diameter=8 mm, class 0.5 from 10 N, class 1 from 2 N to ISO 7500-1, with manufacturers test certificate M according to DIN 55350-18 (Zwick gross force Fₘₐₓ=1.45 kN) (all apparatus from Zwick GmbH & Co. KG, Ulm, Germany) with Order No BTC-FR 2.5 TH. D09 for the tester, Order No BTC-LC 0050N. P01 for the force transducer, Order No BO 70000 S06 for the centring device.

In a preferred embodiment of the invention, the breaking strength is measured by means of a breaking strength tester e.g. Sotax®, type HT100 or type HT1 (Allschwil, Switzerland). Both, the Sotax® HT100 and the Sotax® HT1 can measure the breaking strength according to two different measurement principles: constant speed (where the test jaw is moved at a constant speed adjustable from 5-200 mm/min) or constant force (where the test jaw increases force linearly adjustable from 5-100 N/sec). In principle, both measurement principles are suitable for measuring the breaking strength of the pharmaceutical dosage form according to the invention. Preferably, the breaking strength is measured at constant speed, preferably at a constant speed of 120 mm/min.

In a preferred embodiment, the pharmaceutical dosage form is regarded as being broken if it is fractured into at least two separate pieces.

The pharmaceutical dosage form according to the invention preferably exhibits mechanical strength over a wide temperature range, in addition to the breaking strength (resistance to crushing) optionally also sufficient hardness, impact resistance, impact elasticity, tensile strength and/or modulus of elasticity, optionally also at low temperatures (e.g. below −24° C., below −40° C. or in liquid nitrogen), for it to be virtually impossible to pulverize by spontaneous chewing, grinding in a mortar, pounding, etc. Thus, preferably, the comparatively high breaking strength of the pharmaceutical dosage form according to the invention is maintained even at low or very low temperatures, e.g., when the pharmaceutical dosage form is initially chilled to increase its brittleness, for example to temperatures below −25° C., below −40° C. or even in liquid nitrogen.

The pharmaceutical dosage form according to the invention is characterized by a certain degree of breaking strength. This does not mean that the pharmaceutical dosage form must also exhibit a certain degree of hardness. Hardness and breaking strength are different physical properties. Therefore, the tamper resistance of the pharmaceutical dosage form does not necessarily depend on the hardness of the pharmaceutical dosage form. For instance, due to its breaking strength, impact strength, elasticity modulus and tensile strength, respectively, the pharmaceutical dosage form can preferably be deformed, e.g. plastically, when exerting an external force, for example using a hammer, but cannot be pulverized, i.e., crumbled into a high number of fragments. In other words, the pharmaceutical dosage form according to the invention is characterized by a certain degree of breaking strength, but not necessarily also by a certain degree of form stability.

Therefore, in the meaning of the specification, a pharmaceutical dosage form that is deformed when being exposed to a force in a particular direction of extension but that does not break (plastic deformation or plastic flow) is preferably to be regarded as having the desired breaking strength in said direction of extension.

Preferably, the pharmaceutical dosage form for oral administration
has a breaking strength of at least 400 N, more preferably at least 500 N, still more preferably at least 750 N, yet more preferably at least 1000 N, most preferably at least 1500 N; and/or
comprises an opioid agonist selected from oxycodone and the physiologically acceptable salts thereof; and/or
comprises an opioid antagonist selected from naloxone and the physiologically acceptable salts thereof; and/or
is configured for oral administration twice daily; and/or
contains at least 30 wt.-%, more preferably at least 35 wt.-%, still more preferably at least 40 wt.-% of a polyalkylene oxide having an average molecular weight of at least 500,000 g/mol, more preferably at least 1,000,000 g/mol, relative to the total weight of the pharmaceutical dosage form; and/or
contains a plasticizer, preferably polyethylene glycol; and/or
contains an antioxidant, preferably α-tocopherol; and/or
optionally, contains a free acid, preferably citric acid; and/or
optionally, contains an additional matrix polymer, preferably a cellulose ether, more preferably HPMC.

The pharmaceutical dosage form according to the invention may be produced by different processes, the particularly preferred of which are explained in greater detail below. Several suitable processes have already been described in the prior art. In this regard it can be referred to, e.g., WO 2005/016313, WO 2005/016314, WO 2005/063214, WO 2005/102286, WO 2006/002883, WO 2006/002884, WO 2006/002886, WO 2006/082097, and WO 2006/082099.

The present invention also relates to pharmaceutical dosage forms that are obtainable by any of the processes described here below.

In general, the process for the production of the pharmaceutical dosage form according to the invention preferably comprises the following steps:
(a) mixing all ingredients;
(b) optionally pre-forming the mixture obtained from step (a), preferably by applying heat and/or force to the mixture obtained from step (a), the quantity of heat supplied preferably not being sufficient to heat the polyalkylene oxide up to its softening point;
(c) hardening the mixture by applying heat and force, it being possible to supply the heat during and/or before the application of force and the quantity of heat supplied being sufficient to heat the polyalkylene oxide at least up to its softening point;
(d) optionally singulating the hardened mixture;
(e) optionally shaping the pharmaceutical dosage form; and
(f) optionally providing a film coating.

Heat may be supplied directly, e.g. by contact or by means of hot gas such as hot air, or with the assistance of ultrasound. Force may be applied and/or the pharmaceutical dosage form may be shaped for example by direct tabletting or with the assistance of a suitable extruder, particularly by means of a screw extruder equipped with two screws (twin-screw-extruder) or by means of a planetary gear extruder.

The final shape of the pharmaceutical dosage form may either be provided during the hardening of the mixture by applying heat and force (step (c)) or in a subsequent step (step (e)). In both cases, the mixture of all components is preferably in the plastified state, i.e. preferably, shaping is performed at a temperature at least above the softening point of the polyalkylene oxide. However, extrusion at lower temperatures, e.g. ambient temperature, is also possible and may be preferred.

Shaping can be performed, e.g., by means of a tabletting press comprising die and punches of appropriate shape.

A particularly preferred process for the manufacture of the pharmaceutical dosage form of the invention involves hot-melt extrusion. In this process, the pharmaceutical dosage form according to the invention is produced by thermoforming with the assistance of an extruder, preferably without there being any observable consequent discoloration of the extrudate. It has been surprisingly found that acid is capable of suppressing discoloration. In the absence of acid, the extrudate tends to develop beige to yellowish coloring whereas in the presence of acid the extrudates are substantially colorless, i.e. white.

This process is characterized in that
a) all components are mixed,
b) the resultant mixture is heated in the extruder at least up to the softening point of the polyalkylene oxide and extruded through the outlet orifice of the extruder by application of force,
c) the still plastic extrudate is singulated and formed into the pharmaceutical dosage form or
d) the cooled and optionally reheated singulated extrudate is formed into the pharmaceutical dosage form.

Mixing of the components according to process step a) may also proceed in the extruder.

The components may also be mixed in a mixer known to the person skilled in the art. The mixer may, for example, be a roll mixer, shaking mixer, shear mixer or compulsory mixer.

Before blending with the remaining components, polyalkylene oxide is preferably provided according to the invention with an antioxidant, preferably α-tocopherol. This may proceed by mixing the two components, the polyalkylene oxide and the antioxidant, preferably by dissolving or suspending the antioxidant in a highly volatile solvent and homogeneously mixing this solution or suspension with polyalkylene oxide and removing the solvent by drying, preferably under an inert gas atmosphere.

The, preferably molten, mixture which has been heated in the extruder at least up to the softening point of polyalkylene oxide is extruded from the extruder through a die with at least one bore.

The process according to the invention requires the use of suitable extruders, preferably screw extruders. Screw extruders which are equipped with two screws (twin-screw-extruders) are particularly preferred.

The extrusion is preferably performed so that the expansion of the strand due to extrusion is not more than 30%, i.e. that when using a die with a bore having a diameter of e.g. 6 mm, the extruded strand should have a diameter of not more than 8 mm. More preferably, the expansion of the strand is not more than 25%, still more preferably not more than 20%, most preferably not more than 15% and in particular not more than 10%.

Preferably, extrusion is performed in the absence of water, i.e., no water is added. However, traces of water (e.g., caused by atmospheric humidity) may be present.

The extruder preferably comprises at least two temperature zones, with heating of the mixture at least up to the softening point of the polyalkylene oxide proceeding in the first zone, which is downstream from a feed zone and optionally mixing zone. The throughput of the mixture is preferably from 1.0 kg to 15 kg/hour. In a preferred embodiment, the throughput is from 1 to 3.5 kg/hour. In another preferred embodiment, the throughput is from 4 to 15 kg/hour.

In a preferred embodiment, the die head pressure is within the range of from 25 to 100 bar. The die head pressure can be adjusted inter alia by die geometry, temperature profile and extrusion speed.

The die geometry or the geometry of the bores is freely selectable. The die or the bores may accordingly exhibit a round, oblong or oval cross-section, wherein the round cross-section preferably has a diameter of 0.1 mm to 15 mm and the oblong cross-section preferably has a maximum lengthwise extension of 21 mm and a crosswise extension of 10 mm. Preferably, the die or the bores have a round cross-section. The casing of the extruder used according to the invention may be heated or cooled. The corresponding temperature control, i.e. heating or cooling, is arranged in such a way that the mixture to be extruded exhibits at least an average temperature (product temperature) corresponding to the softening temperature of the polyalkylene oxide and does not rise above a temperature at which the opioid agonist to be processed may be damaged. Preferably, the temperature of the mixture to be extruded is adjusted to below 180° C., preferably below 150° C., but at least to the softening temperature of polyalkylene oxide. Typical extrusion temperatures are 120° C., 130° C. and 135° C.

In a preferred embodiment, the extruder torque is within the range of from 30 to 95%. Extruder torque can be adjusted inter alia by die geometry, temperature profile and extrusion speed.

After extrusion of the molten mixture and optional cooling of the extruded strand or extruded strands, the extrudates are preferably singulated. This singulation may preferably be performed by cutting up the extrudates by means of revolving or rotating knives, water jet cutters, wires, blades or with the assistance of laser cutters.

Preferably, intermediate or final storage of the optionally singulated extrudate or the final shape of the pharmaceutical dosage form according to the invention is performed under oxygen-free atmosphere which may be achieved, e.g., by means of oxygen-scavengers.

The singulated extrudate may be press-formed into tablets in order to impart the final shape to the pharmaceutical dosage form.

The application of force in the extruder onto the at least plasticized mixture is adjusted by controlling the rotational speed of the conveying device in the extruder and the geometry thereof and by dimensioning the outlet orifice in such a manner that the pressure necessary for extruding the plasticized mixture is built up in the extruder, preferably immediately prior to extrusion. The extrusion parameters which, for each particular composition, are necessary to give rise to a pharmaceutical dosage form with desired mechanical properties, may be established by simple preliminary testing.

For example but not limiting, extrusion may be performed by means of a twin-screw-extruder type ZSE 18 or ZSE27 (Leistritz, Nurnberg, Germany), screw diameters of 18 or 27 mm. Screws having eccentric ends may be used. A heatable die with a round bore having a diameter of 4, 5, 6, 7, 8, or 9 mm may be used. The extrusion parameters may be adjusted e.g. to the following values: rotational speed of the screws: 120 Upm; delivery rate 2 kg/h for a ZSE 18 or 8 kg/h for a ZSE27; product temperature: in front of die 125° C. and behind die 135° C.; and jacket temperature: 110° C.

Preferably, extrusion is performed by means of twin-screw-extruders or planetary-gear-extruders, twin-screw extruders (co-rotating or contra-rotating) being particularly preferred.

The pharmaceutical dosage form according to the invention is preferably produced by thermoforming with the assistance of an extruder without any observable consequent discoloration of the extrudates.

The process for the preparation of the pharmaceutical dosage form according to the invention is preferably performed continuously. Preferably, the process involves the extrusion of a homogeneous mixture of all components. It is particularly advantageous if the thus obtained intermediate, e.g. the strand obtained by extrusion, exhibits uniform properties. Particularly desirable are uniform density, uniform distribution of the active compound, uniform mechanical properties, uniform porosity, uniform appearance of the surface, etc. Only under these circumstances the uniformity of the pharmacological properties, such as the stability of the release profile, may be ensured and the amount of rejects can be kept low.

A further aspect of the invention relates to the use of an opioid agonist in combination with an opioid antagonist for the manufacture of the pharmaceutical dosage form as described above for the treatment of pain, preferably moderate to severe pain such as moderate to severe low back pain.

A further aspect of the invention relates to the use of a pharmaceutical dosage form as described above for avoiding or hindering the abuse of the opioid agonist contained therein.

A further aspect of the invention relates to the use of a pharmaceutical dosage form as described above for avoiding or hindering the unintentional overdose of the opioid agonist contained therein.

In this regard, the invention also relates to the use of a opioid agonist as described above and/or a opioid antagonist as described above and/or a polyalkylene oxide as described above for the manufacture of the pharmaceutical dosage form according to the invention for the prophylaxis and/or the treatment of a disorder, thereby preventing an overdose of the opioid agonist, particularly due to comminution of the pharmaceutical dosage form by mechanical action.

Further, the invention relates to a method for the prophylaxis and/or the treatment of a disorder comprising the administration of the pharmaceutical dosage form according to the invention, thereby preventing an overdose of the opioid agonist, particularly due to comminution of the pharmaceutical dosage form by mechanical action. Preferably, the mechanical action is selected from the group consisting of chewing, grinding in a mortar, pounding, and using apparatuses for pulverizing conventional pharmaceutical dosage forms.

The following examples further illustrate the invention but are not to be construed as limiting its scope.

### General Procedure:

Polyethylene oxide, α-tocopherol, oxycodone hydrochloride, naloxone hydrochloride and all other excipients were weighted and sieved to each other.

The powder was mixed and dosed gravimetrically to an extruder. Hot-melt extrusion was performed by means of a twin screw extruder of type ZSE18 PH 40D (Leistritz, Nurnberg, Germany) that was equipped with a heatable round die having a diameter of 5, 7, 8 or 9 mm.

The hot extrudate was cooled by ambient air and the cooled extrusion strand was comminuted to cut pieces. The cut pieces were shaped by means of an excenter press which was equipped with punches of various size and shape.

The breaking strength of the pharmaceutical dosage forms was measured by means of a Sotax® HT100. A tablet was regarded as failing the breaking strength test when during the measurement the force dropped below the threshold value of 25% of the maximum force that was observed during the measurement, regardless of whether the dosage form was fractured into separate pieces or not. All values are given as a mean of 10 measurements.

The in vitro release profile of the pharmacologically active ingredient (Oxycodone HCl and Naloxone HCl) was measured in 600 ml or 900 mL of blank FeSSIF (pH 5.0) at temperature of 37° C. with sinker (type 1 or 2). The rotation speed of the paddle was adjusted to 150/min. The pharmacologically active ingredient was detected by means of a spectrometric measurement with a wavelength of 218 nm.

Other in vitro release profiles of the pharmacologically active ingredient (Oxycodone HCl and Naloxone HCl or Hydromorphone HCl and Naloxone HCl) were measured in 500 ml of simulated gastric fluid (SGFsp, sp=sine pancreatine, i.e. without enzyme) at temperature of 37° C. with sinker (type 1 or 2). The rotation speed of the paddle was adjusted to 75/min. The pharmacologically active ingredient was detected by means of a spectrometric measurement with a wavelength of 218 nm.

Further in vitro release profiles of the pharmacologically active ingredient (Oxycodone HCl and Naloxone HCl or Hydromorphone HCl and Naloxone HCl) were measured in 500 ml of ethanol (40%) at temperature of 37° C. with sinker (type 1 or 2). The rotation speed of the paddle was adjusted to 75/min. The pharmacologically active ingredient was detected by means of a spectrometric measurement with a wavelength of 218 nm.

### EXAMPLE I

Tablets having the following composition were prepared:

| | |
|---|---|
| Oxycodone HCl | 14.39% |
| Naloxone HCl | 7.19% |
| Polyethylene Oxide M_{w} 7.000.000 | 56.30% |
| Polyethylene Glycol 6000 | 13.44% |
| HPMC | 7.64% |
| Alpha Tocopherol | 0.20% |
| Citric acid, anhydrous | 0.84% |
| Tablet weight | 278 mg |

Tablets were prepared by using the following punches:

| Example | Form of punch |
|---|---|
| Example 1-1 | biconcave, round, diameter 9 mm, radius of curvature 7.2 mm |
| Example 1-2 | biconvex, round, diameter 9 mm, radius of curvature 9/1 mm |
| Example 1-3 | biconvex, round, diameter 9 mm, radius of curvature 15/1 mm |
| Example 1-4 | biconcave, pentagonal, diameter 9 mm, radius of |
| | curvature 7.2 mm |

All tablets did not break at a force of 1000 N, the upper measuring limit of the testing device.

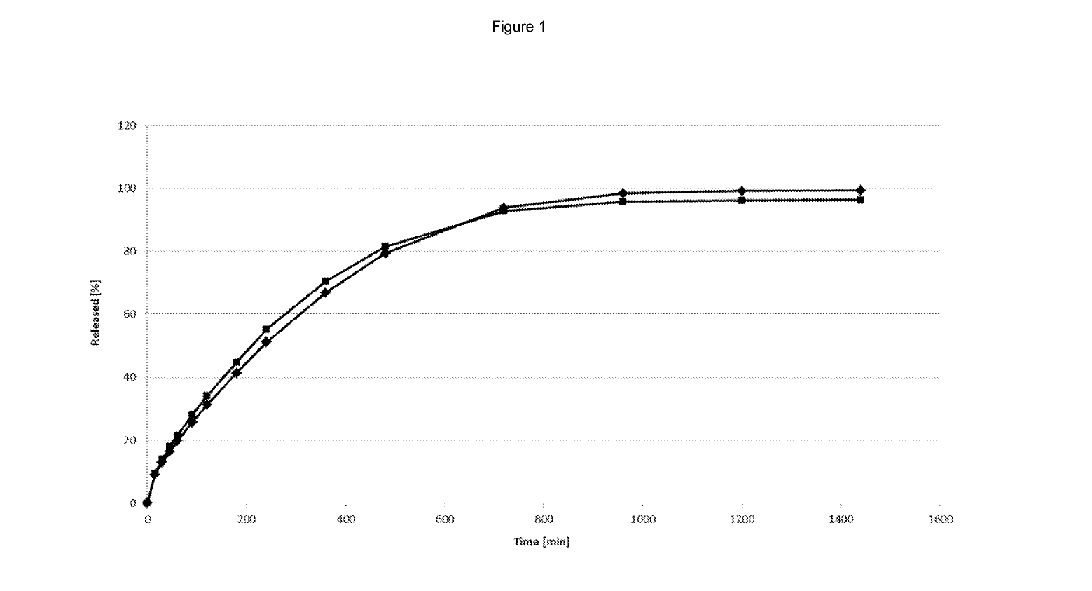

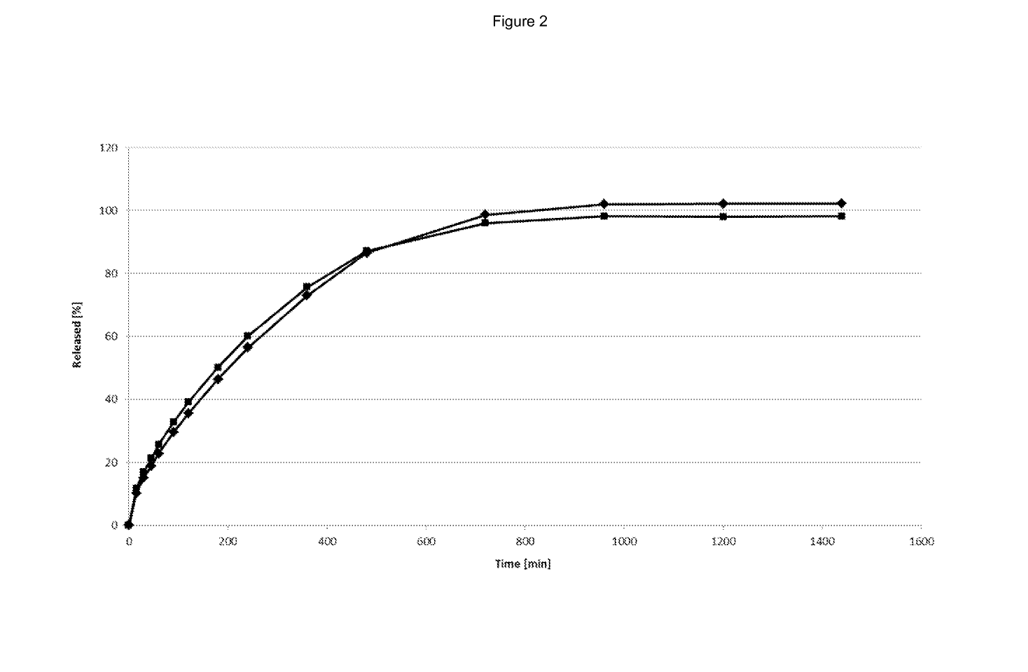

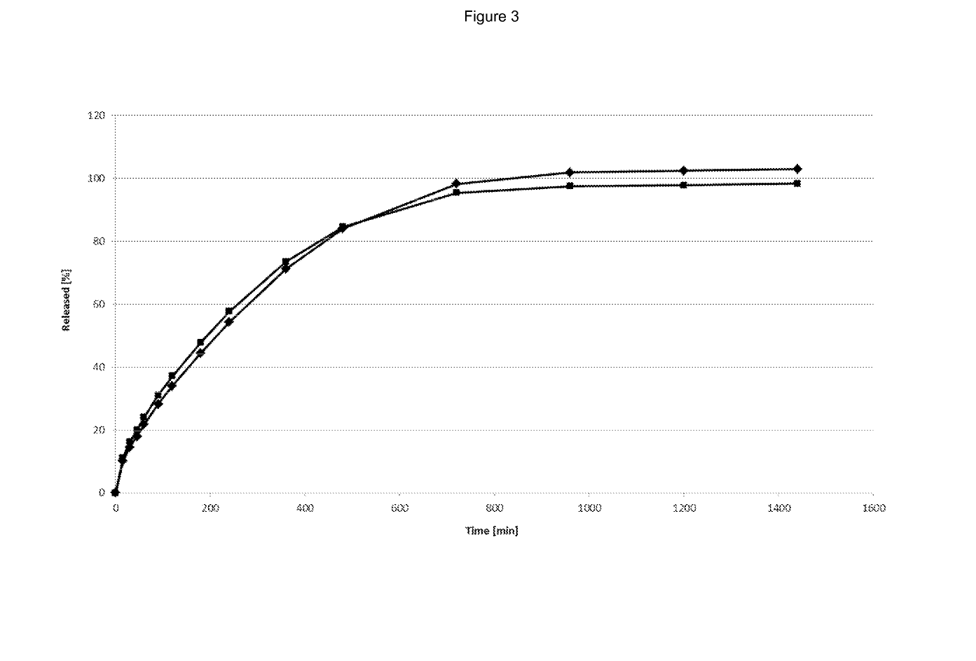

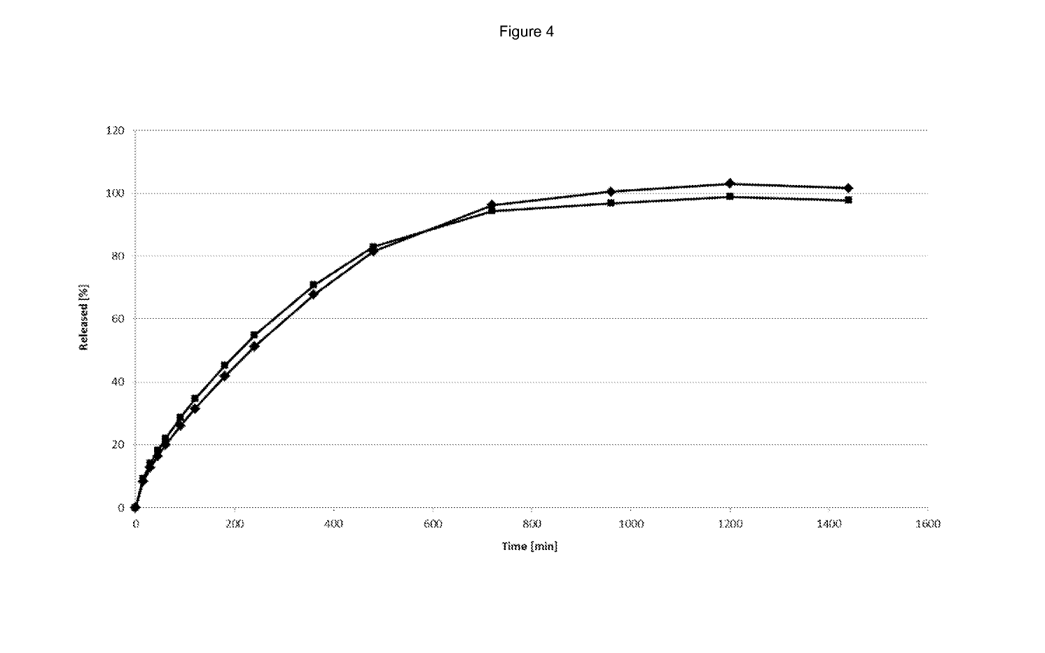

The in vitro release profiles of the pharmaceutical dosage forms according to Examples 1-1 to 1-4 are displayed in FIGS. 1 to 4:
FIG. 1: Example 1-1 ▪ drug release Oxycodone; ♦ drug release Naloxone
FIG. 2: Example 1-2 ▪ drug release Oxycodone; ♦ drug release Naloxone
FIG. 3: Example 1-3: ▪ drug release Oxycodone; ♦ drug release Naloxone
FIG. 4: Example 1-4 ▪ drug release Oxycodone; ♦ drug release Naloxone

As can be seen, the in vitro release profile of the opioid agonist essentially corresponds to the in vitro release profile of the opioid antagonist.

### EXAMPLE II

Tablets having the following composition were prepared:

| | |
|---|---|
| Oxycodone HCl | 14.39% |
| Naloxone HCl | 7.19% |
| Polyethylene Oxide M_{w} 7.000.000 | 53.82% |
| Polyethylene Glycol 6000 | 13.56% |
| HPMC | 10.00% |
| Alpha Tocopherol | 0.20% |
| Citric acid, anhydrous | 0.84% |
| Tablet weight | 278 mg |

Tablets were prepared by using the following punches:

| Example | Form of punch |
|---|---|
| Example 2-1 | biconcave, round, diameter 9 mm, radius of curvature 7.2 mm |
| Example 2-2 | biconvex, round, diameter 9 mm, radius of curvature 9/1 mm |
| Example 2-3 | biconvex, round, diameter 9 mm, radius of curvature 15/1 mm |
| Example 2-4 | biconcave, pentagonal, diameter 9 mm, radius of |
| | curvature 7.2 mm |
| Example 2-5 | Biconvex, oblong, 6 mm × 15 mm |
| Example 2-6 | Biconvex, oblong, 6.4 mm × 13.6 mm |

All tablets did not break at a force of 1000 N, the upper measuring limit of the testing device.

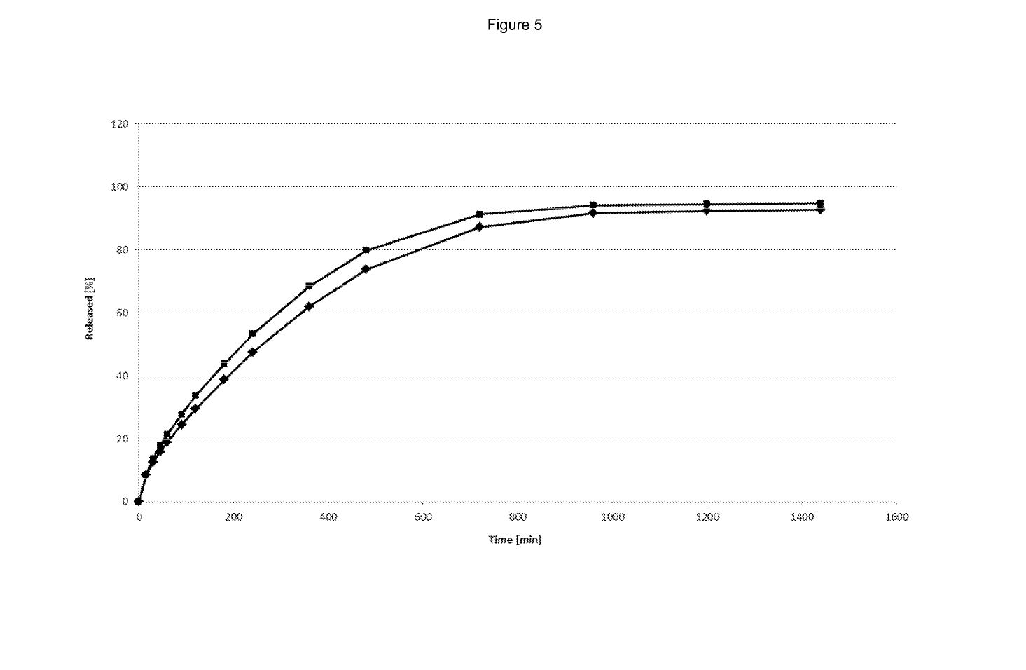

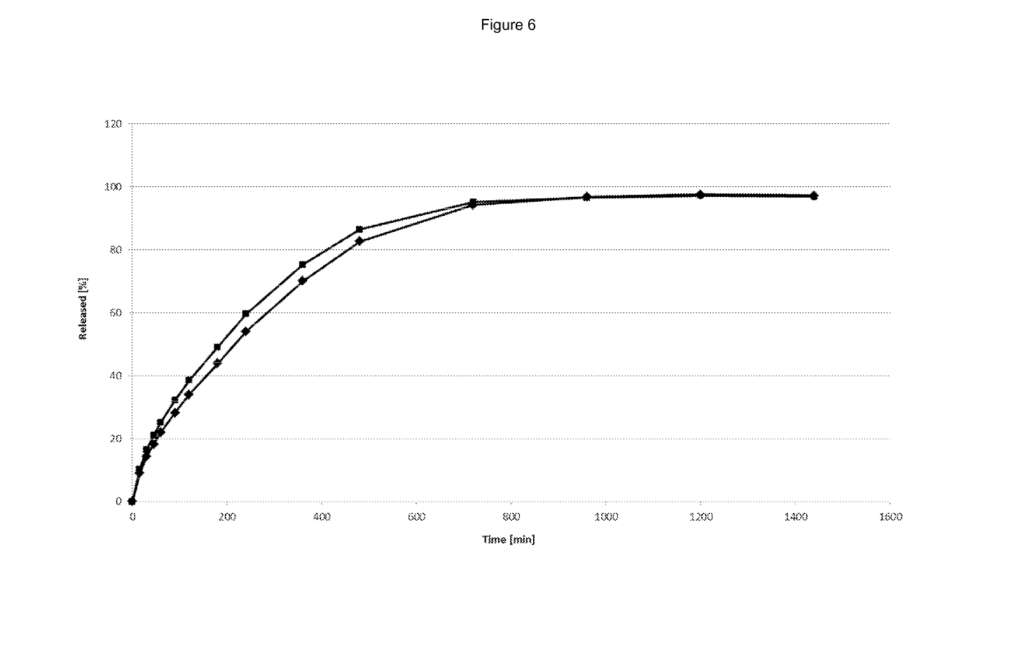

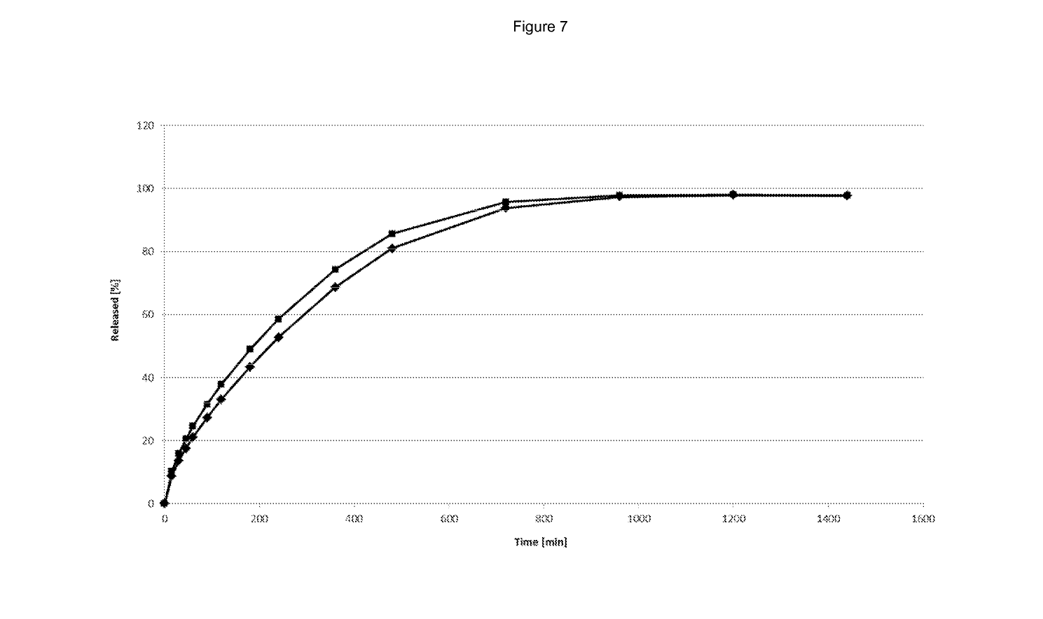

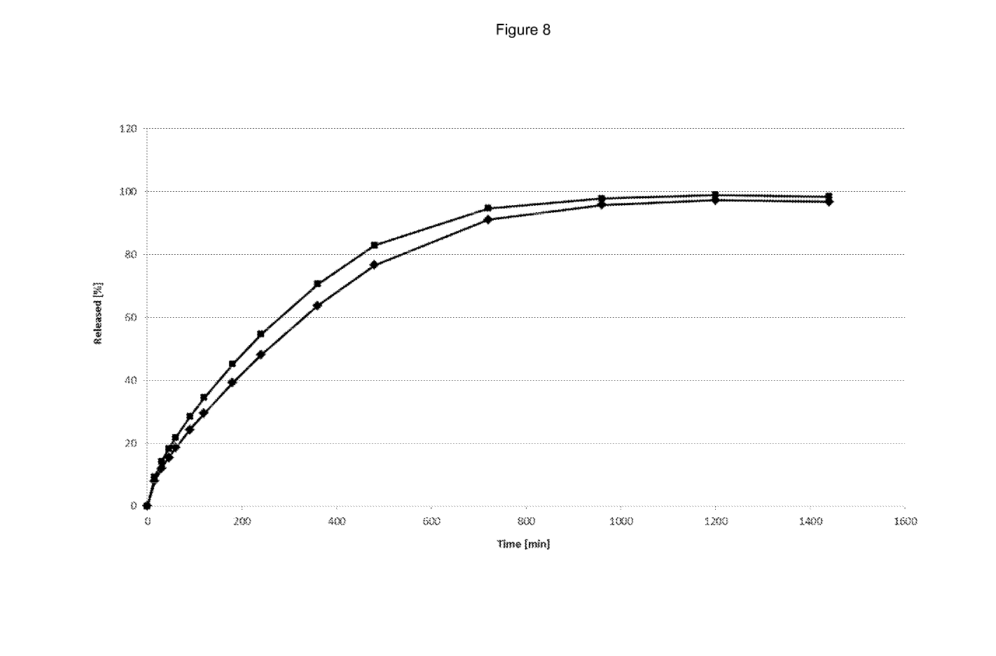

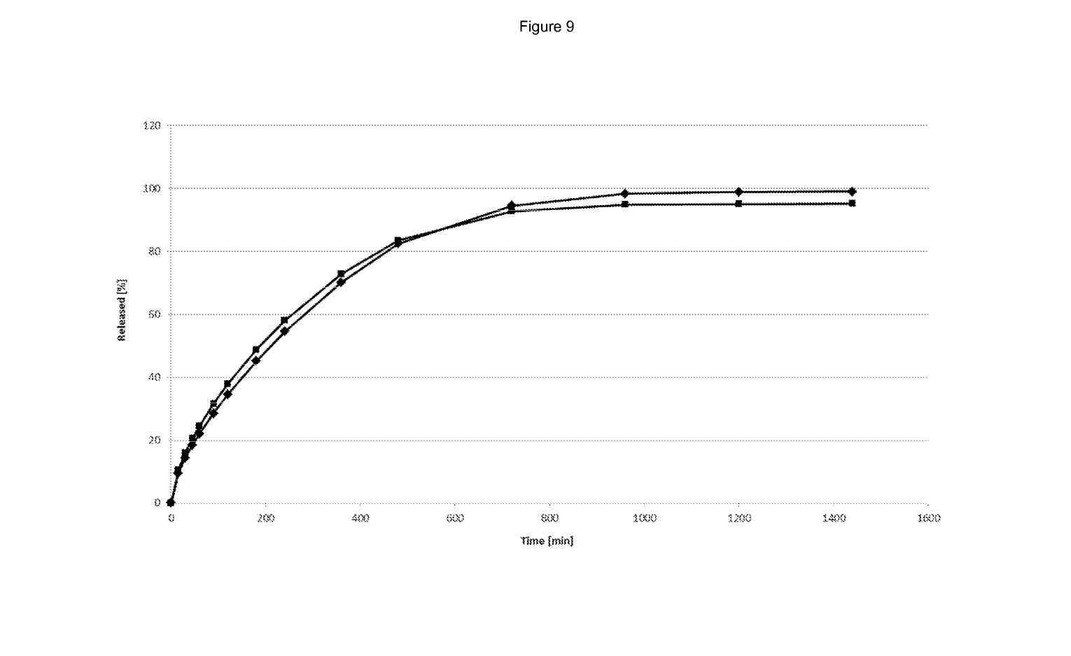

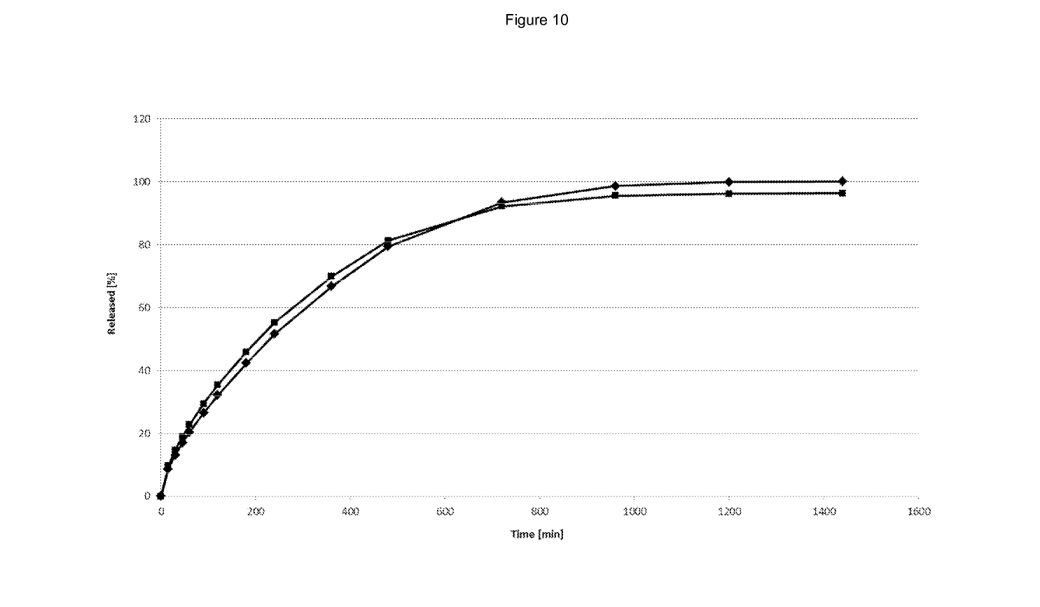

The in vitro release profiles of the pharmaceutical dosage forms according to Examples 2-1 to 2-6 are displayed in FIGS. 5 to 10:
FIG. 5: Example 2-1 ▪ drug release Oxycodone; ♦ drug release Naloxone
FIG. 6: Example 2-2 ▪ drug release Oxycodone; ♦ drug release Naloxone
FIG. 7: Example 2-3 ▪ drug release Oxycodone; ♦ drug release Naloxone
FIG. 8: Example 2-4 ▪ drug release Oxycodone; ♦ drug release Naloxone
FIG. 9: Example 2-5: ▪ drug release Oxycodone; ♦ drug release Naloxone
FIG. 10: Example 2-6 ▪ drug release Oxycodone; ♦ drug release Naloxone

As can be seen, the in vitro release profile of the opioid agonist essentially corresponds to the in vitro release profile of the opioid antagonist.

### EXAMPLE III

Tablets having the following composition were prepared:

| | |
|---|---|
| Oxycodone HCl | 16.00% |
| Naloxone HCl | 8.00% |
| Polyethylene Oxide M_{w} 7.000.000 | 62.60% |
| Polyethylene Glycol 6000 | 3.75% |
| HPMC | 8.50% |
| Alpha Tocopherol | 0.22% |
| Citric acid, anhydrous | 0.93% |
| Tablet weight | 250 mg |

Tablets were prepared by using the following punches:

| Example | Form of punch |
|---|---|
| Example 3-1 | biconcave, round, diameter 9 mm, radius of curvature 7.2 mm |
| Example 3-2 | biconvex, round, diameter 9 mm, radius of curvature 15/1 mm |
| Example 3-3 | Biconvex, oblong, 6 mm × 15 mm |

All tablets did not break at a force of 1000 N, the upper measuring limit of the testing device.

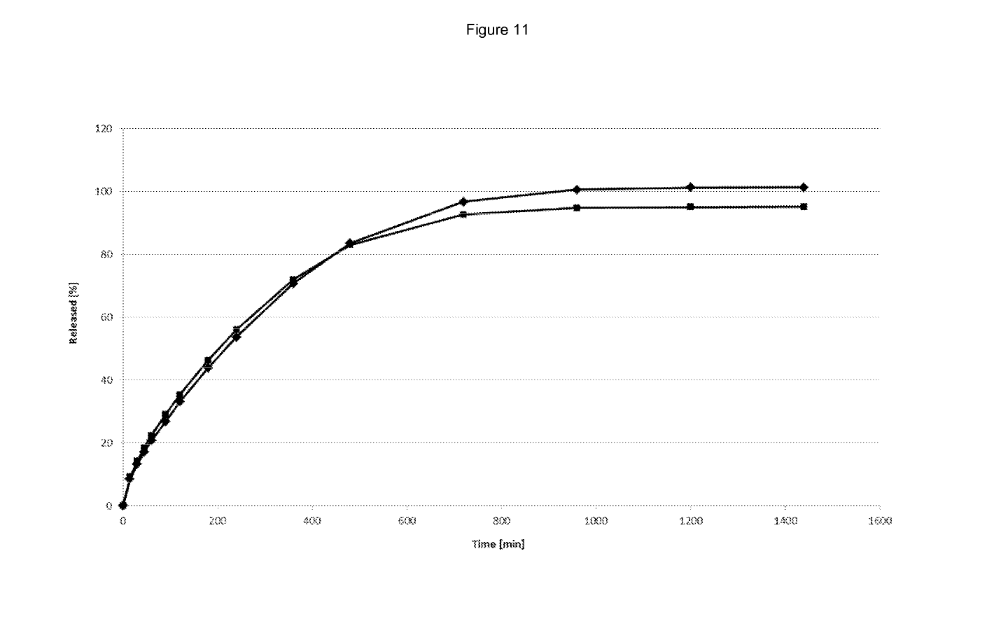

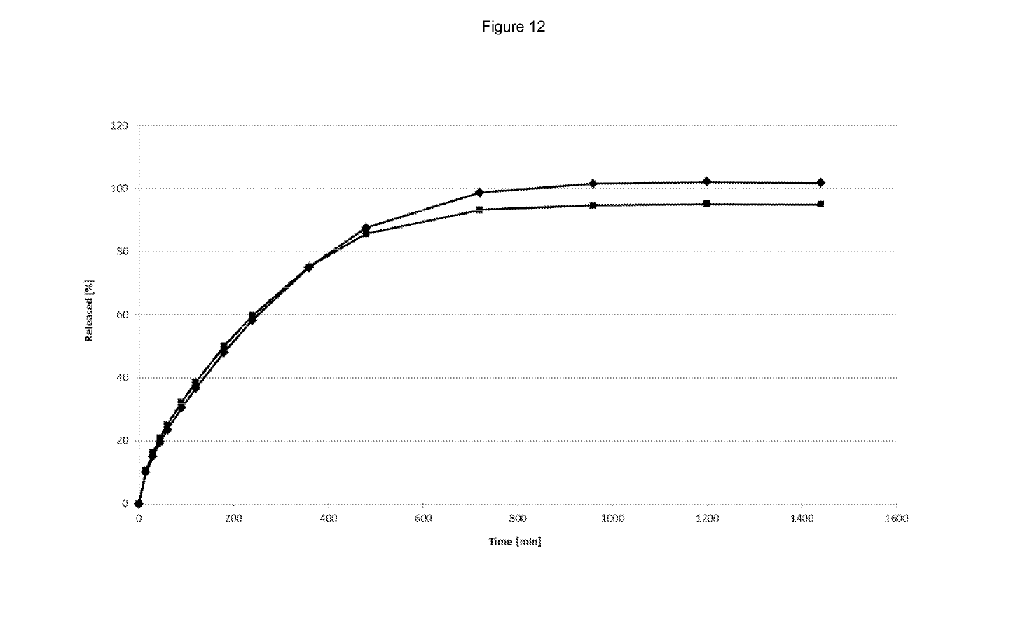

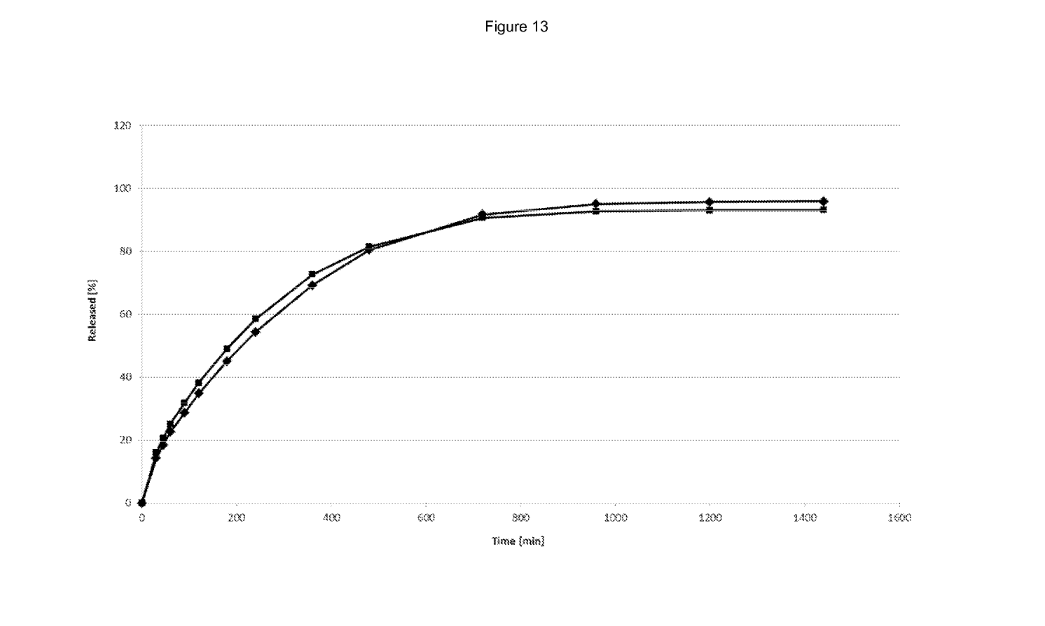

The in vitro release profiles of the pharmaceutical dosage forms according to Examples 3-1 to 3-3 are displayed in FIGS. 11 to 13:
FIG. 11: Example 3-1 ▪ drug release Oxycodone; ♦ drug release Naloxone
FIG. 12: Example 3-2 ▪ drug release Oxycodone; ♦ drug release Naloxone
FIG. 13: Example 3-3 ▪ drug release Oxycodone; ♦ drug release Naloxone

As can be seen, the in vitro release profile of the opioid agonist essentially corresponds to the in vitro release profile of the opioid antagonist.

### EXAMPLE IV

Tablets having the following composition were prepared:

| | |
|---|---|
| Oxycodone HCl | 16.00% |
| Naloxone HCl | 8.00% |
| Polyethylene Oxide M_{w} 7.000.000 | 45.00% |
| Polyethylene Glycol 6000 | 15.00% |
| HPMC | 15.00% |
| Alpha Tocopherol | 0.20% |
| Citric acid, anhydrous | 0.80% |
| Tablet weight | 250 mg |

Tablets were prepared by using the following punches:

| Example | Form of punch |
|---|---|
| Example 4-1 | biconcave, round, diameter 9 mm, radius of curvature |
| | 7.2 mm |
| Example 4-2 | biconvex, round, diameter 9 mm, radius of curvature |
| | 15/1 mm |
| Example 4-3 | Biconvex, oblong, 6 mm × 15 mm |

All tablets did not break at a force of 1000 N, the upper measuring limit of the testing device.

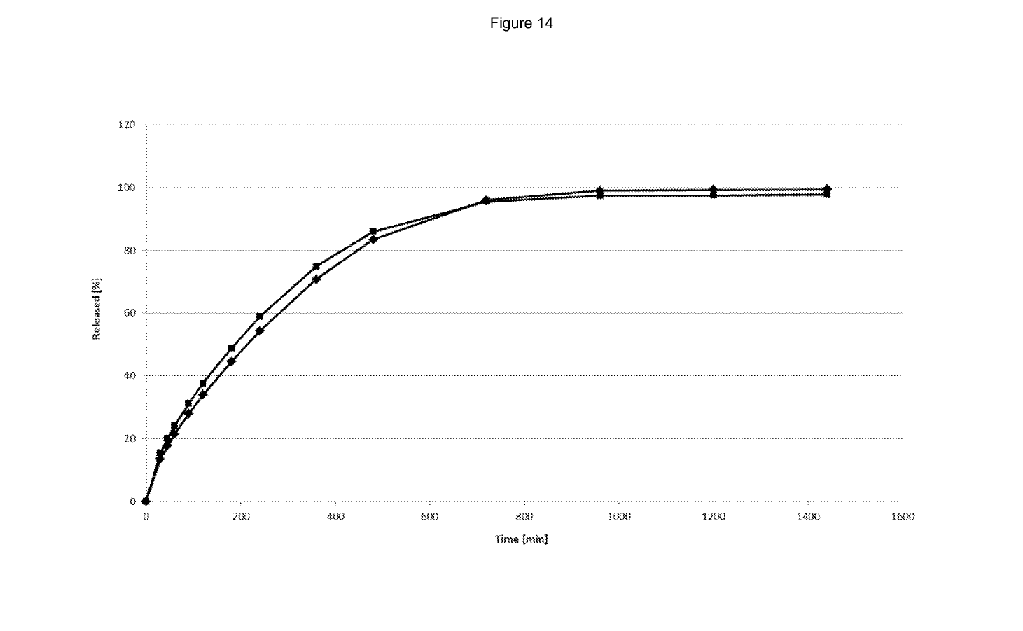

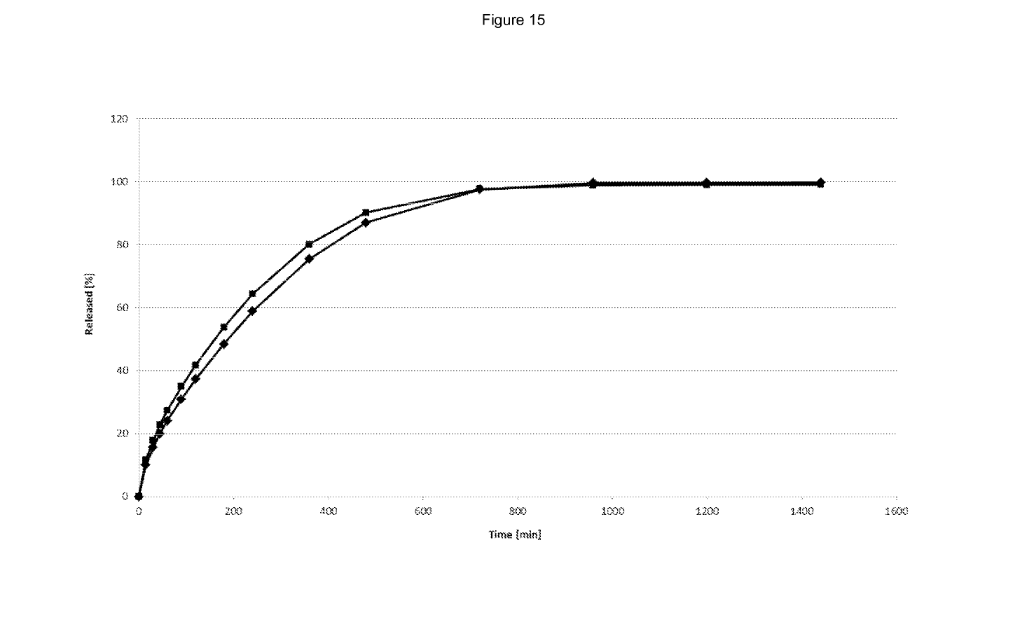

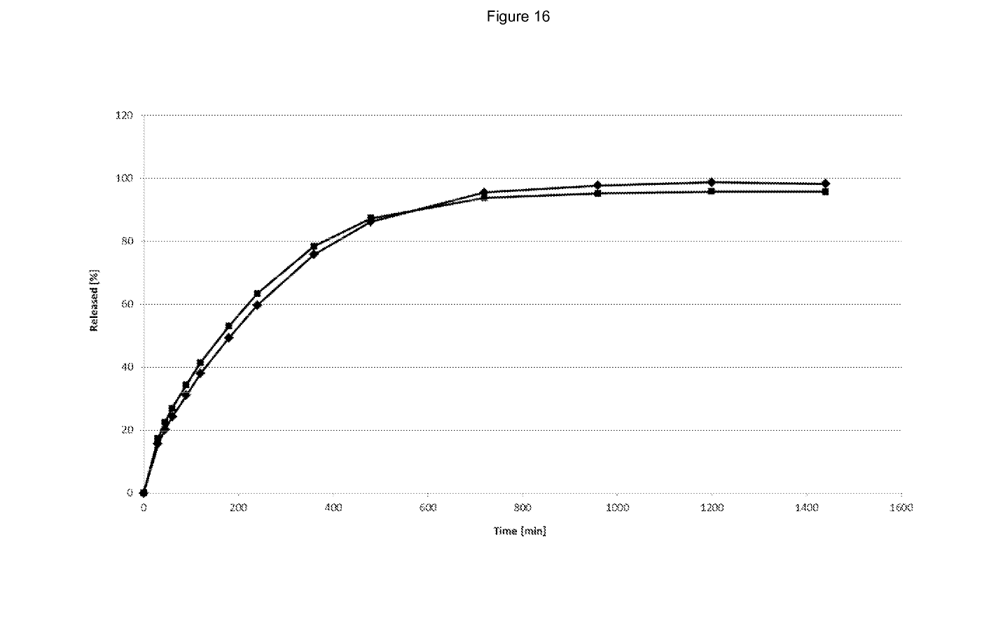

The in vitro release profiles of the pharmaceutical dosage forms according to Examples 4-1 to 4-3 are displayed in FIGS. 14 to 16:
FIG. 14: Example 4-1 ▪ drug release Oxycodone; ♦ drug release Naloxone
FIG. 15: Example 4-2 ▪ drug release Oxycodone; ♦ drug release Naloxone
FIG. 16: Example 4-3 ▪ drug release Oxycodone; ♦ drug release Naloxone

As can be seen, the in vitro release profile of the opioid agonist essentially corresponds to the in vitro release profile of the opioid antagonist.

### EXAMPLE V

Tablets of Examples 3-3 and 4-3 as well as those of commercially available Targin 40/20 tablets were investigated in respect to their dissolution robustness in different media. The analytical method was used as described above, but the dissolution medium was either phosphate buffer pH 6.8, hydrochloric acid 0.1N pH 1.2, or ethanol 40% (v/v).

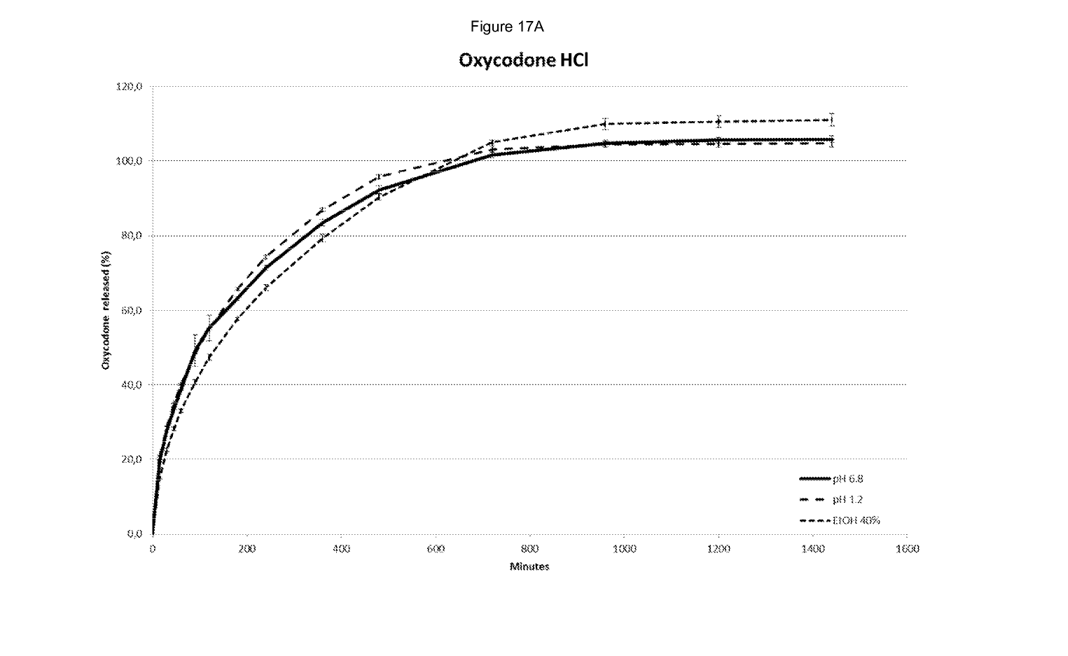

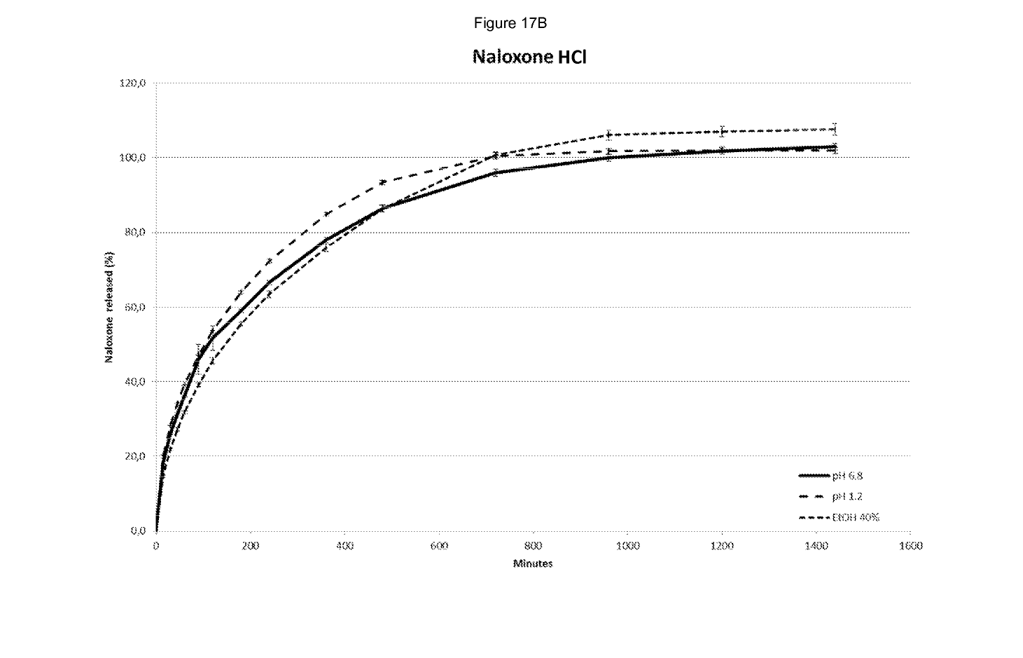

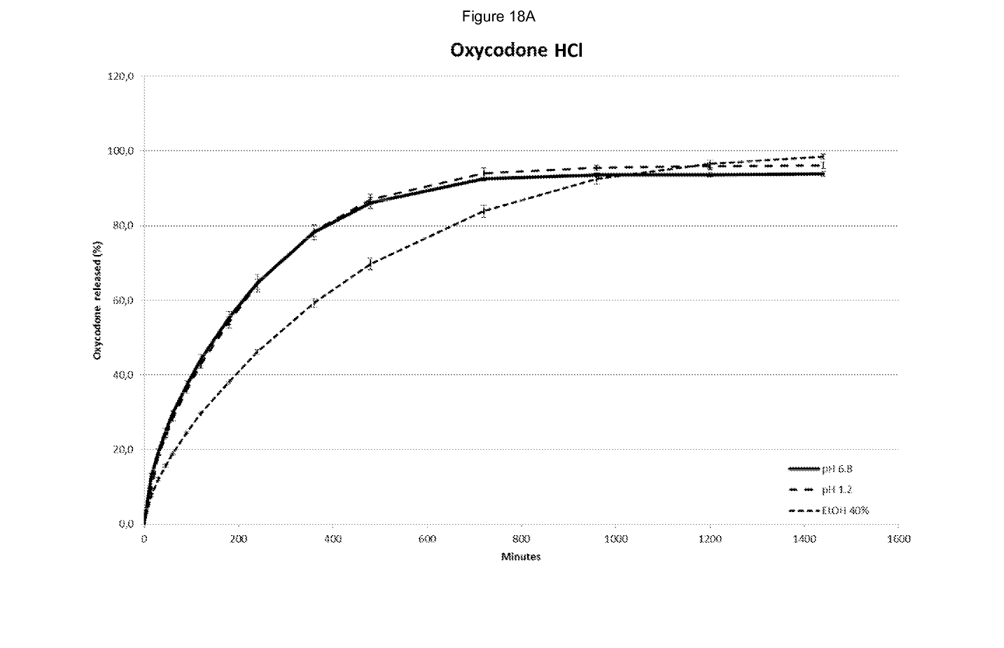

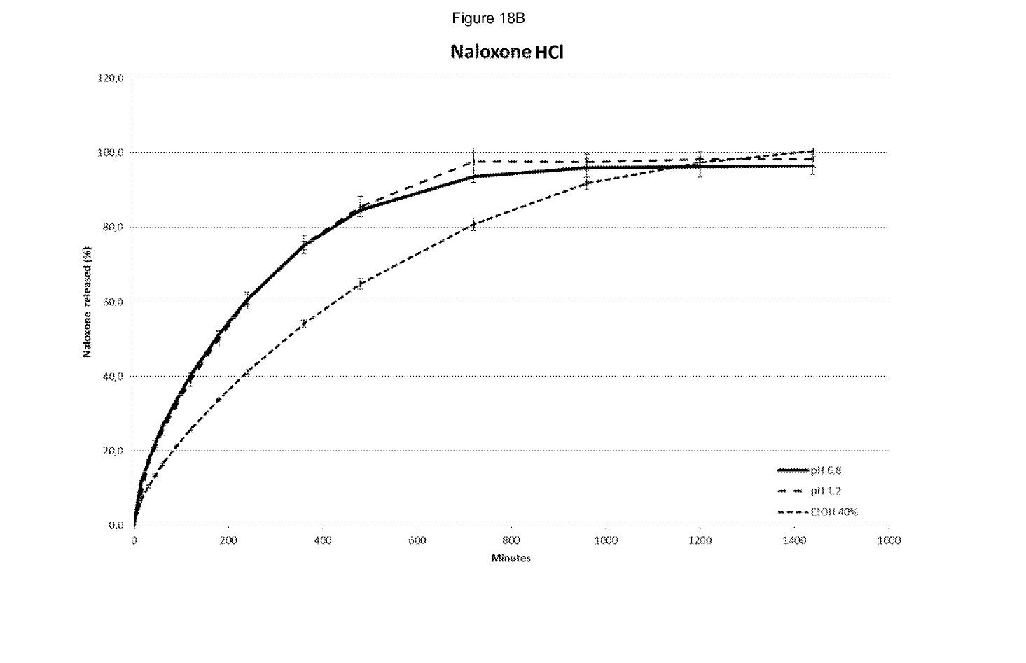

In FIGS. 17 to 19B the resulting dissolution profiles are depicted.

For the commercially available Targin tablets (FIGS. 17A and 17B) the dissolution of the antagonist naloxone is slower than that of the agonist oxycodone in any dissolution medium. The dissolution rate is constant in acidic and ethanolic medium in comparison to the phosphate buffer.

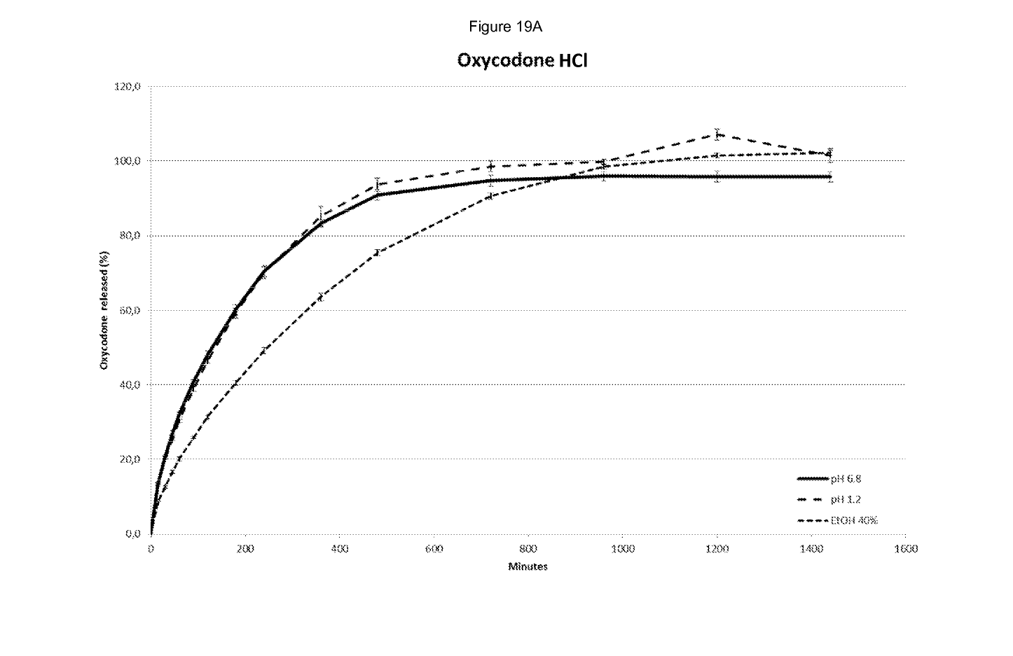

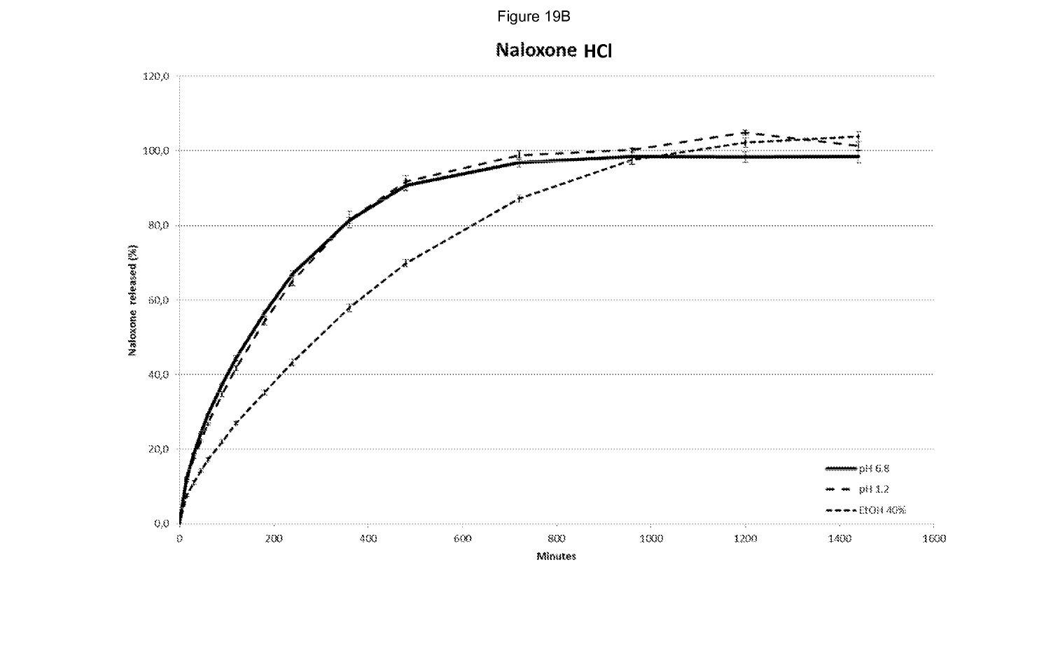

For the inventive formulations 3-3 (FIGS. 18A and 18B) and 4-3 (FIGS. 19A and 19B) a significant improvement could be achieved. The dissolution speed of the antagonist and the agonist is the same. Dissolution in acidic medium has the same speed as in phosphate buffer. In ethanolic medium dissolution of both antagonist and agonist is slower than in the other media.

Thus the inventive formulations are superior over the commercial product, as abuse is impeded by a higher dissolution rate of the antagonist in all media and by a decreased dissolution in ethanolic media.

Extraction in ethanol 40% (v/v) was further tested in an extraction trails. A tablets were put into 30 mL of ethanol 40% (v/v) and shaken at room temperature for 30 minutes. The amount of both oxycodone and naloxone was determined in the supernatant using HPLC. The results are as follows:

From Targin 13.9% of oxycodone and 13.8% of naloxone could be extracted. For inventive formulation 3-3 only 7.9% of oxycodone and 7.2% of naloxone could be extracted, for inventive formulation 4-3 the extracted amounts were measured to 7.6% oxycodone and 6.7% naloxone.

The inventive formulations are superior to the commercial product due to the lower amount of drug extracted.

### EXAMPLE VI

Tablets having the following compositions were prepared:

| | Example | Example | Example | Example | Example | Example |
|---|---|---|---|---|---|---|
| | 6-1 | 6-2 | 6-3 | 6-4 | 6-5 | 6-6 |
| Oxycodone HCl | 29.5% | 15.2% | 9.9% | 26.5% | 6.1% | 1.2% |
| Naloxone HCl | 14.7% | 7.6% | 4.9% | 13.3% | 3.1% | 0.6% |
| Polyethylene Oxide | 55.0% | 55.0% | 55.0% | 30.0% | 90.0% | 55.0% |
| M_{w} 7.000.000 | | | | | | |
| Polyethylene Glycol | 0.6%** | 12.0% | 12.0% | 12.0% | 0.6%** | 25.0% |
| (Macrogol) 6000 | | | | | | |
| HPMC | — | 10.0% | 18.0% | 18.0% | — | 18.0% |
| 100,000 mPa · s | | | | | | |
| Alpha Tocopherol* | 0.1% | 0.1% | 0.1% | 0.1% | 0.1% | 0.1% |
| Citric acid, | 0.1% | 0.1% | 0.1% | 0.1% | 0.1% | 0.1% |
| anhydrous | | | | | | |
| Tablet weight | 600 mg | 600 mg | 600 mg | 600 mg | 600 mg | 600 mg |

*weighed in as Macrogol 6000/14% alpha tocopherol mixture
**already contained in Macrogol 6000/14% alpha tocopherol mixture

Tablets were prepared by using a nozzle with a diameter of 9 mm and the following punch:

Form of punch
biconvex, round, diameter 11 mm, radius of curvature 8.8 mm

All tablets did not break at a force of 1000 N, the upper measuring limit of the testing device.

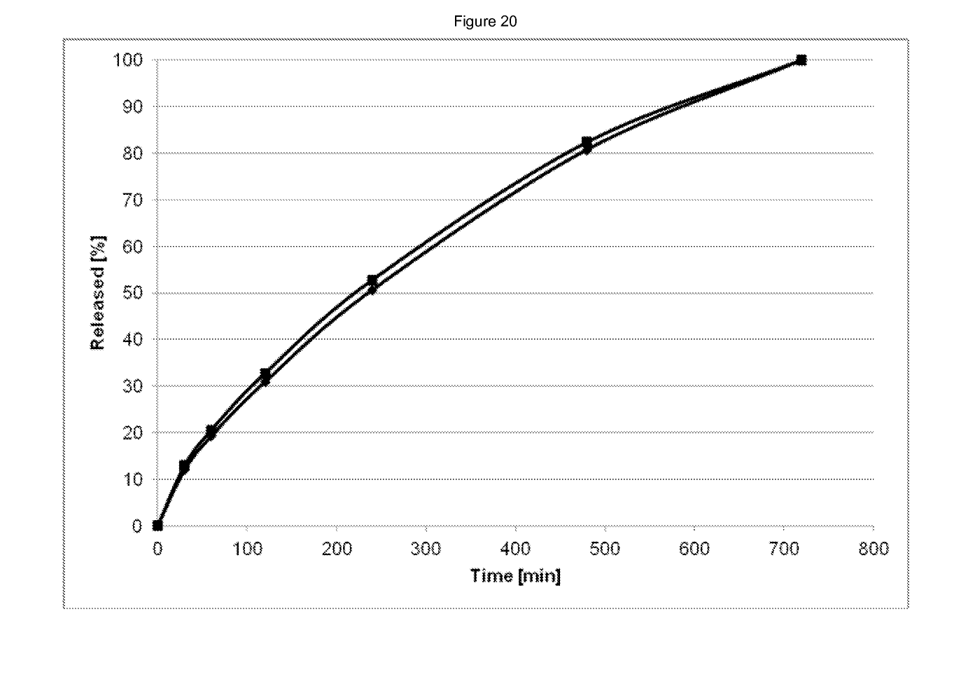

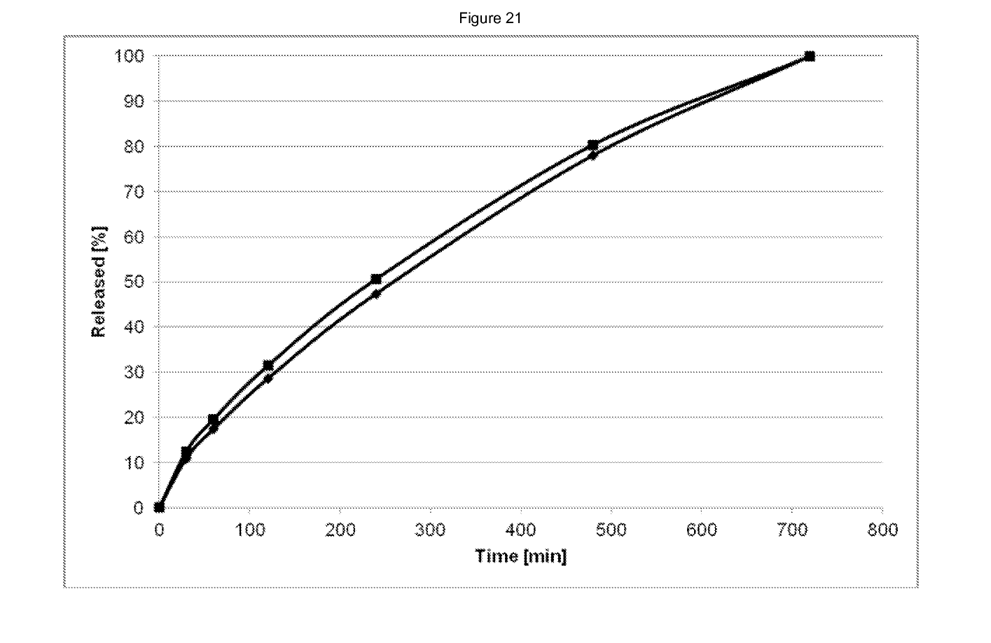

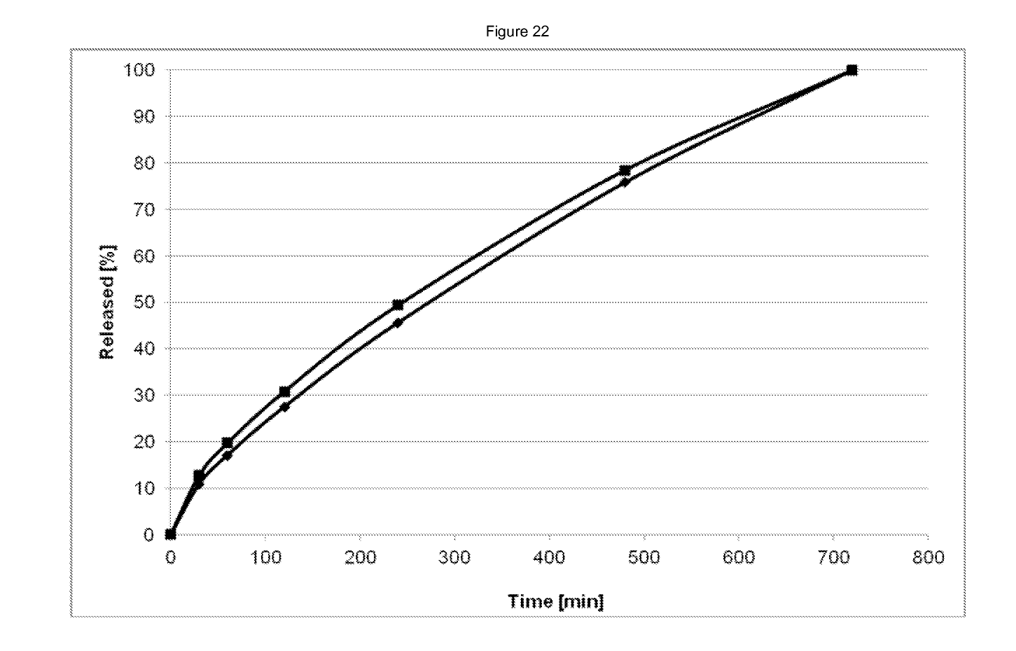

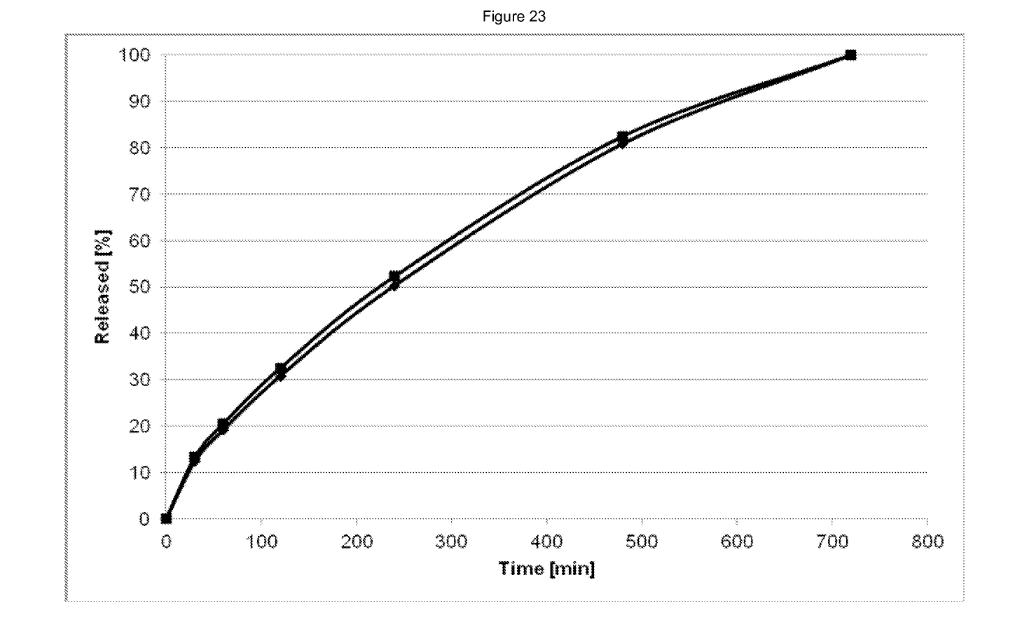

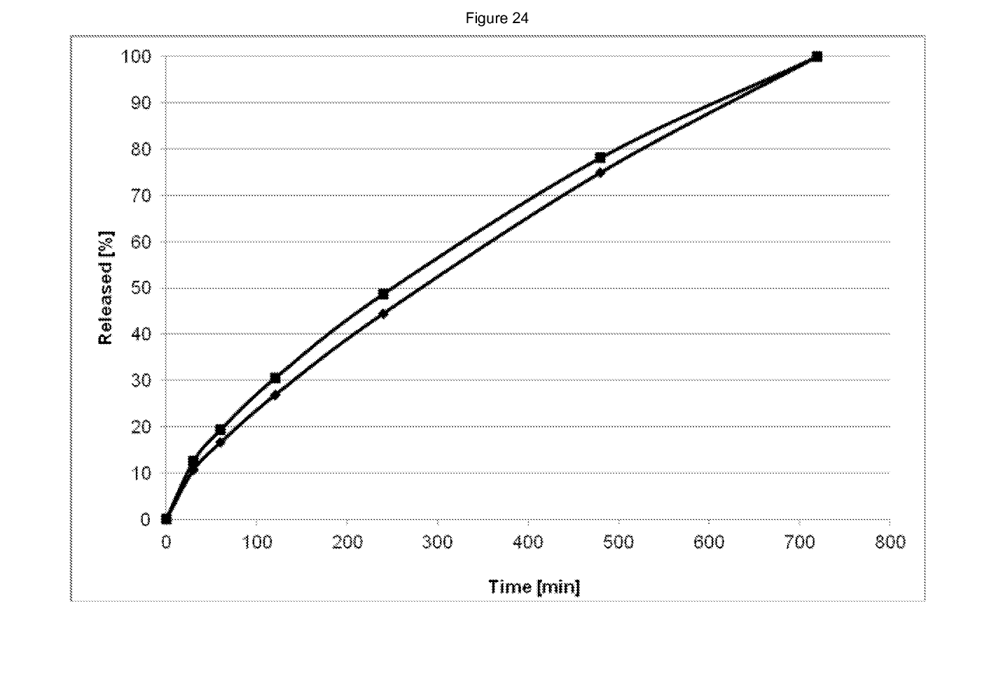

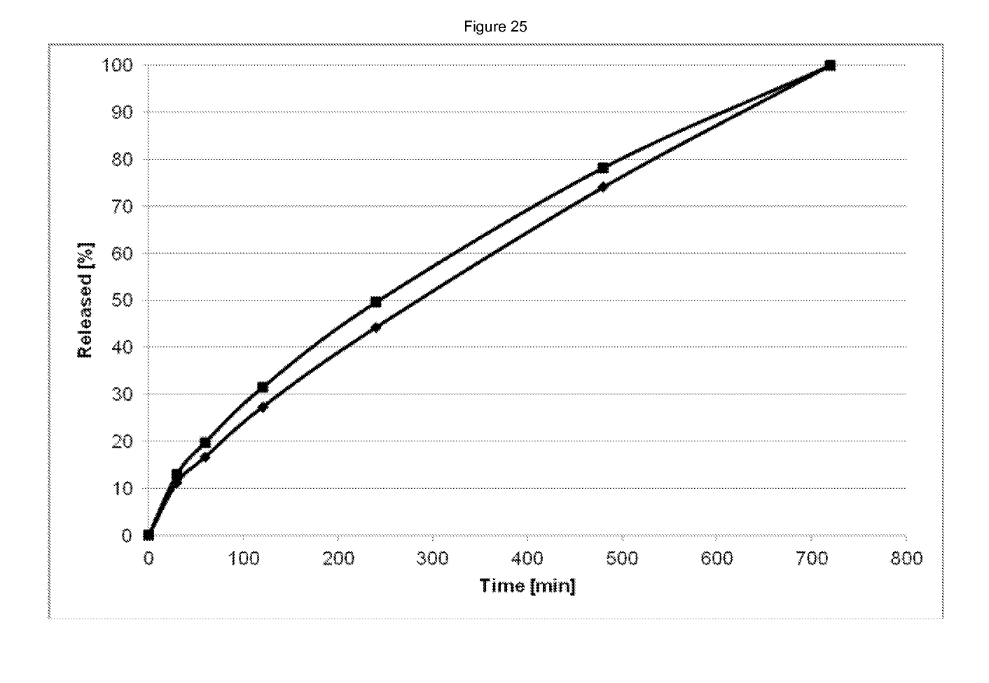

The in vitro release profiles at pH 5 (normalized values) of the pharmaceutical dosage forms according to Examples 6-1 to 6-6 are displayed in FIGS. 20 to 25:
FIG. 20: Example 6-1 ▪ drug release Oxycodone; ♦ drug release Naloxone
FIG. 21: Example 6-2 ▪ drug release Oxycodone; ♦ drug release Naloxone
FIG. 22: Example 6-3 ▪ drug release Oxycodone; ♦ drug release Naloxone
FIG. 23: Example 6-4 ▪ drug release Oxycodone; ♦ drug release Naloxone
FIG. 24: Example 6-5 ▪ drug release Oxycodone; ♦ drug release Naloxone
FIG. 25: Example 6-6 ▪ drug release Oxycodone; ♦ drug release Naloxone

As can be seen, the in vitro release profile of the opioid agonist essentially corresponds to the in vitro release profile of the opioid antagonist.

### EXAMPLE VII

Tablets in accordance with WO 2010/14007 (examples 5 and 4) having the following compositions were prepared:

| | Comparative | Comparative |
|---|---|---|
| | Example 7-1 | Example 7-2 |
| Hydromorphone HCl | 1.4% | 1.4% |
| Naloxone HCl | 2.8% | 2.8% |
| Polyacrylate dispersion 40% | 14.0% | 14.0% |
| Ethylcellulose N10 | 9.0% | 9.0% |
| HPMC 5 mPa · s | 0.1% | 0.1% |
| Glycerol monostearate | 0.7% | 0.7% |
| Talcum | 7.0% | 7.0% |
| Stearyl alcohol | 1.8% | 1.8% |
| Glycerol dibehenate | 1.1% | 1.1% |
| Lactose anhydrous | 21.4% | 21.4% |
| Polyethylene Oxide M_{w} 4.000.000 | 39.6% | — |
| HPMC 100,000 mPa · s | — | 39.6% |
| Magnesium stearate | 1.1% | 1.1% |
| Tablet weight | 285.13 mg | 285.13 mg |

Further, tablets having the following compositions were prepared:

| | Example 7-3 |
|---|---|
| Hydromorphone HCl | 1.4% |
| Naloxone HCl | 2.8% |
| Polyethylene Oxide M_{w} 7.000.000 | 56.7% |
| Polyethylene Glycol (Macrogol) | 18.9% |
| 6000 | |
| HPMC 100,000 mPa · s | 18.9% |
| Alpha Tocopherol* | 0.2% |
| Citric acid, anhydrous | 1.0% |
| Tablet weight | 285 mg |

*weighed in as Macrogol 6000/14% alpha tocopherol mixture

Tablets according to Comparative Examples 7-1 and 7-2 were prepared from a powder mixture which was mixed with hydromorphone/naloxone pellets/granulates. Tablets according to Example 7-3 were prepared from a powder mixture which was extrudated using a die with a diameter of 8 mm.

For the preparation of all tablets the following punch was used:

Form of punch
biconvex, round, diameter 9 mm, radius of curvature 7.2 mm

Tablets according to Comparative Examples 7-1 and 7-2 had a breaking strength of 23 N (Comp. Ex. 7-1) and 34 N (Comp. Ex. 7-2), respectively, and could be crushed with spoons.

Tablets according to Example 7-3 did not break at a force of 1000 N, the upper measuring limit of the testing device, and could not be manipulated with spoons.

The in vitro release profiles of the manipulated and the intact tablets were determined in simulated gastric fluid (SGFsp) and ethanol (40%), respectively.

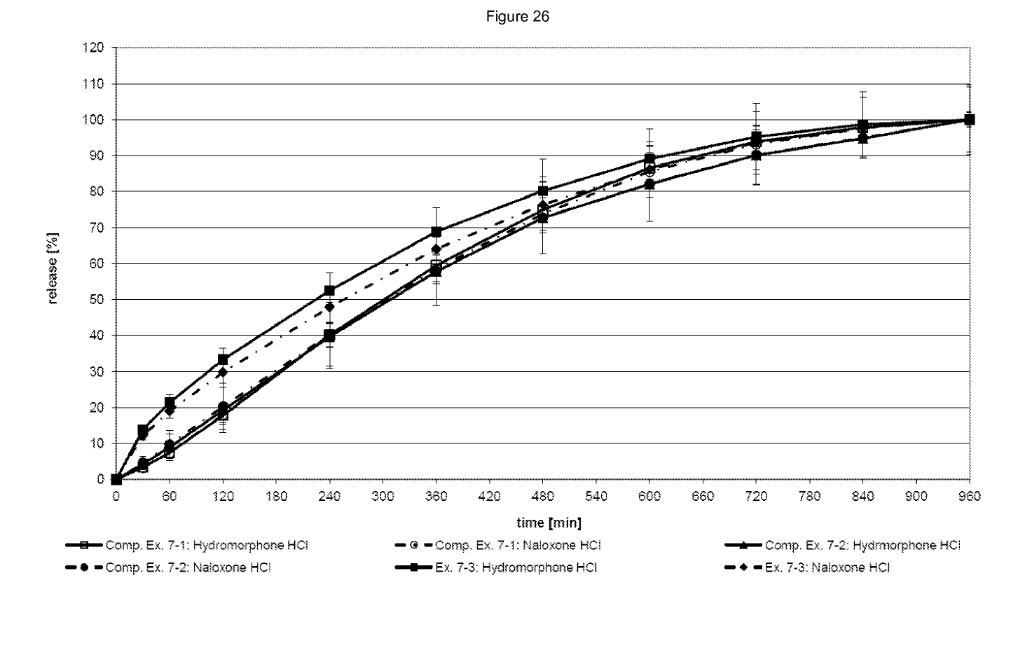

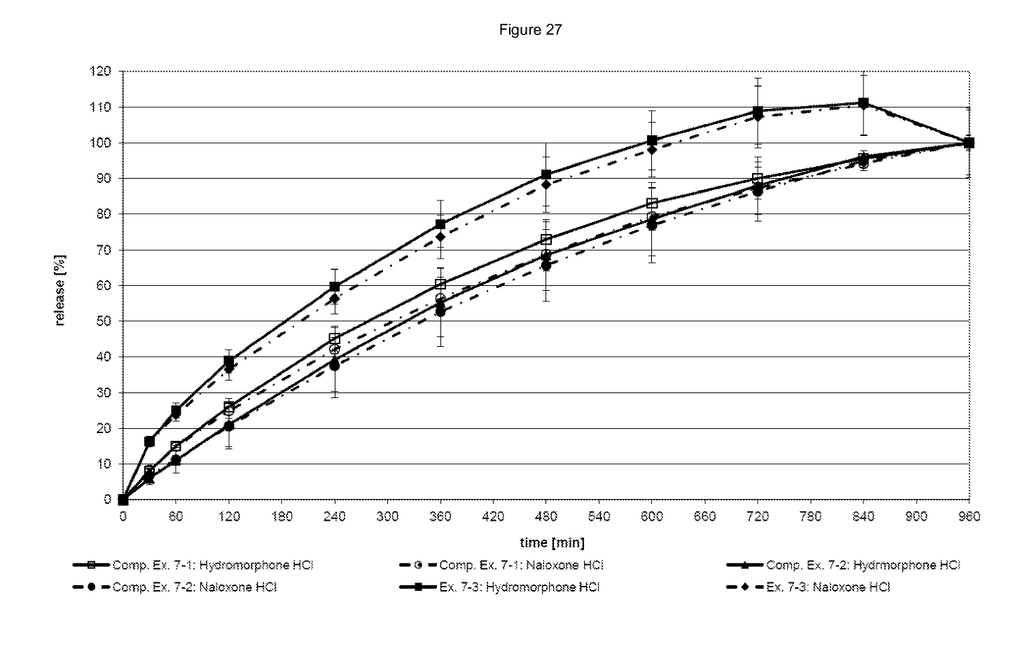

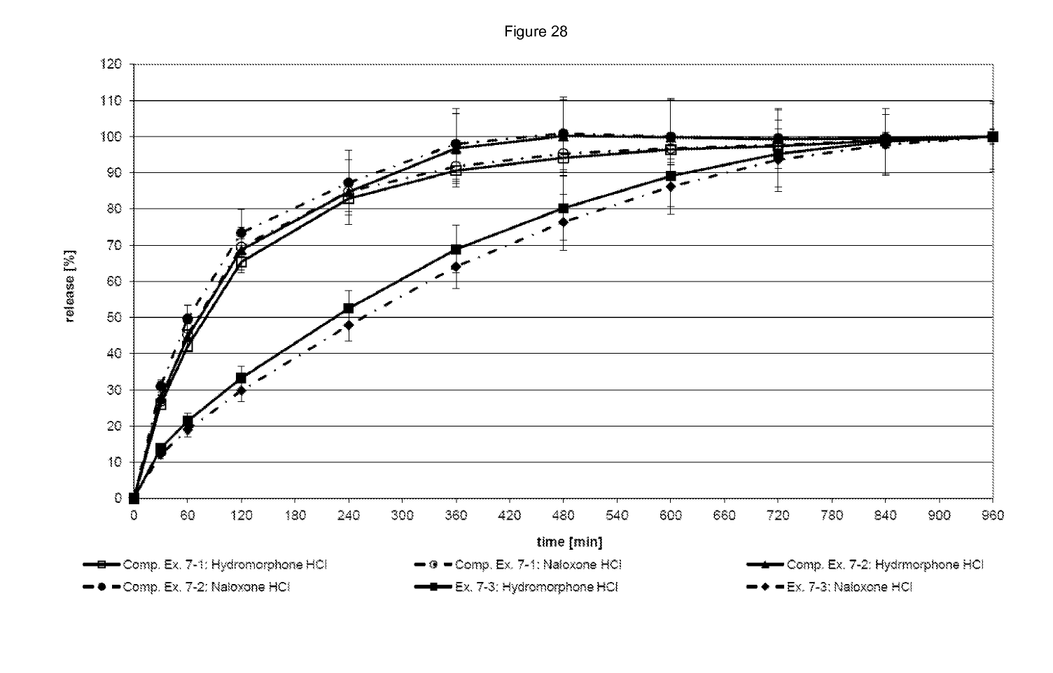

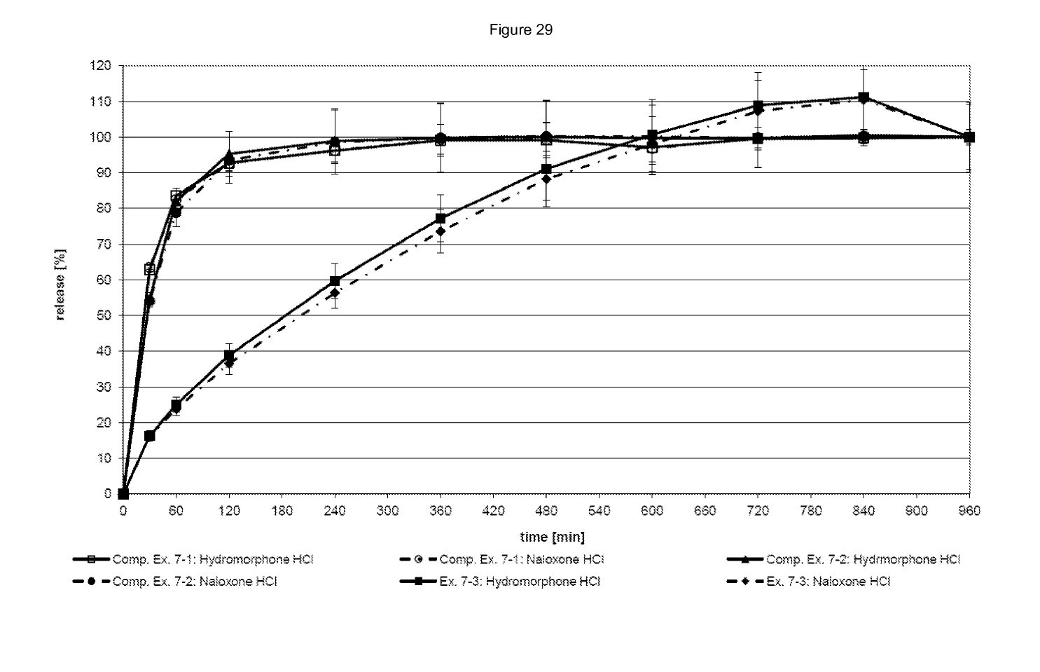

The in vitro release profiles (normalized values) of the pharmaceutical dosage forms according to Examples 7-1 to 7-3 are displayed in FIGS. 26 to 29:
FIG. 26: drug release of hydromorphone HCl and naloxone HCl of intact tablets in simulated gastric fluids
FIG. 27: drug release of Hydromorphone and Naloxone of intact tablets in 40% ethanol
FIG. 28: drug release of Hydromorphone and Naloxone of manipulated tablets in simulated gastric fluids
FIG. 29: drug release of Hydromorphone and Naloxone of manipulated tablets in 40% ethanol

As can be seen, the manipulated tablets according to Comparative Examples 7-1 and 7-2, respectively, lost their controlled release properties.

The manipulation of the tablets according to Example 7-3 had neither an effect on the shape of the tablets nor on their release properties.

### EXAMPLE VIII

Tablets in accordance with WO 2010/14007 (analogous to examples 5 and 4) having the following compositions were prepared:

| | Comparative | Comparative |
|---|---|---|
| | Example 8-1 | Example 8-2 |
| Oxycodone HCl | 1.8% | 1.8% |
| Naloxone HCl | 0.9% | 0.9% |
| Polyacrylate dispersion 40% | 14.6% | 14.6% |
| Ethylcellulose N10 | 9.4% | 9.4% |
| HPMC 5 mPa · s | 0.1% | 0.1% |
| Glycerol monostearate | 0.7% | 0.7% |
| Talcum | 7.3% | 7.3% |
| Stearyl alcohol | 1.8% | 1.8% |
| Glycerol dibehenate | 1.1% | 1.1% |
| Lactose anhydrous | 21.4% | 21.4% |
| Polyethylene Oxide M_{w} 4.000.000 | 39.6% | — |
| HPMC 100,000 mPa · s | — | 39.6% |
| Magnesium stearate | 1.2% | 1.2% |
| Tablet weight | 273.77 mg | 273.77 mg |

Further, tablets having the following compositions were prepared:

| | Example 8-3 |
|---|---|
| Oxycodone HCl | 1.8% |
| Naloxone HCl | 0.9% |
| Polyethylene Oxide M_{w} 7.000.000 | 57.6% |
| Polyethylene Glycol (Macrogol) | 19.2% |
| 6000 | |
| HPMC 100,000 mPa · s | 19.2% |
| Alpha Tocopherol* | 0.2% |
| Citric acid, anhydrous | 1.0% |
| Tablet weight | 285 mg |

*weighed in as Macrogol 6000/14% alpha tocopherol mixture

Tablets according to Comparative Examples 8-1 and 8-2 were prepared from a powder mixture which was mixed with hydromorphone/naloxone pellets/granulates. Tablets according to Example 8-3 were prepared from a powder mixture which was extrudated using a die with a diameter of 8 mm.

For the preparation of all tablets the following punch was used:

Form of punch
biconvex, round, diameter 9 mm, radius of curvature 7.2 mm

Tablets according to Comparative Examples 8-1 and 8-2 had a breaking strength of 16 N (Comp. Ex. 8-1) and 32 N (Comp. Ex. 8-2), respectively, and could be crushed with spoons.

Tablets according to Example 8-3 did not break at a force of 1000 N, the upper measuring limit of the testing device, and could not be manipulated with spoons.

The in vitro release profiles of the manipulated and the intact tablets were determined in simulated gastric fluid (SGFsp) and ethanol (40%), respectively.

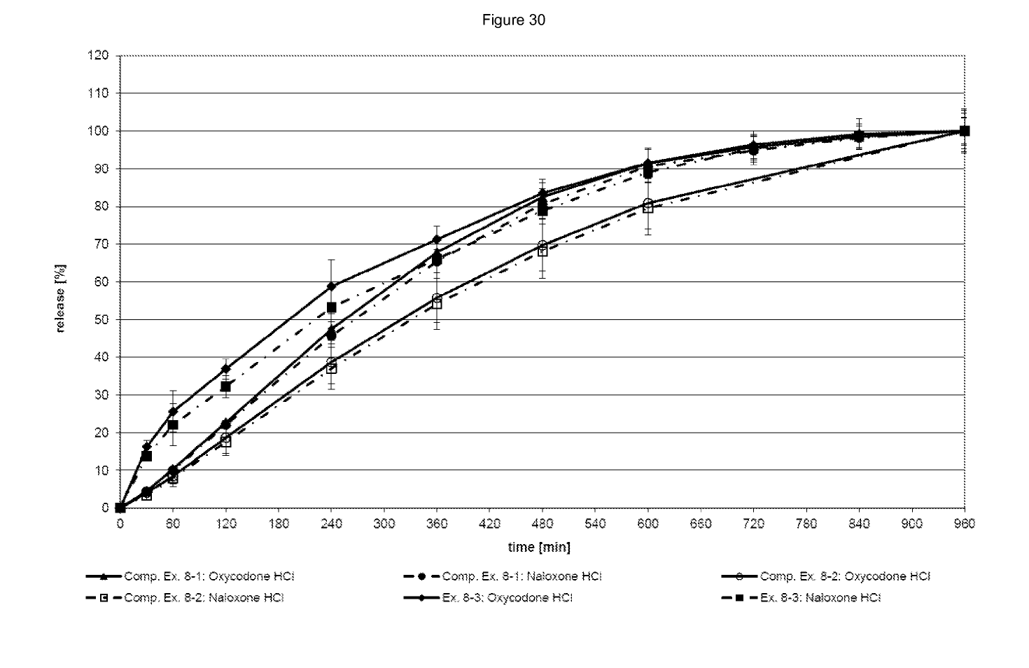

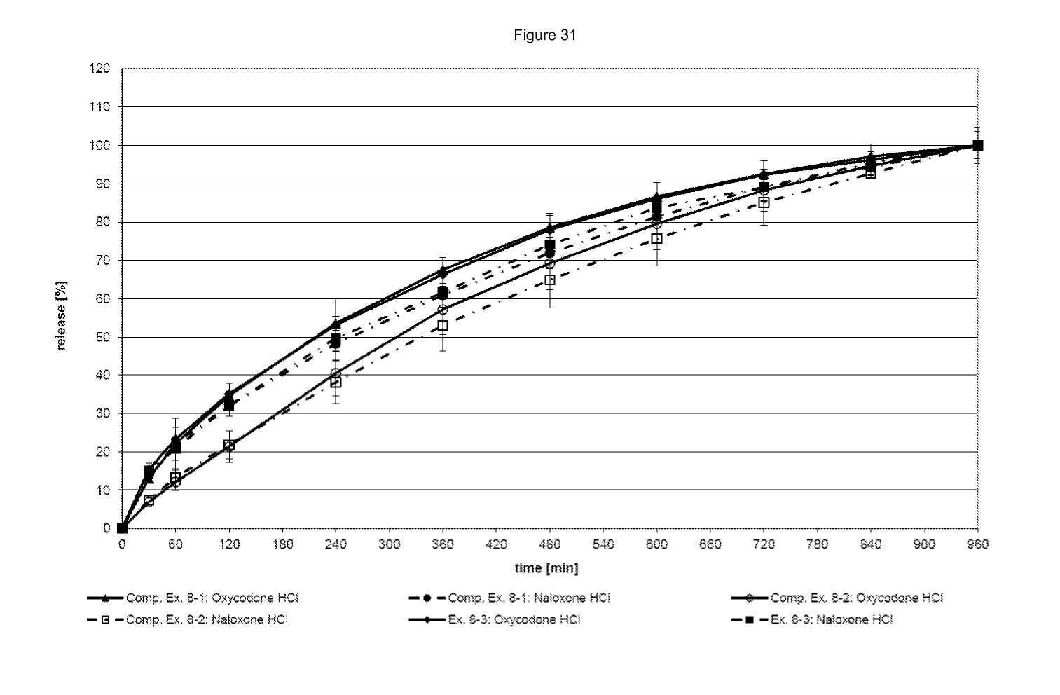

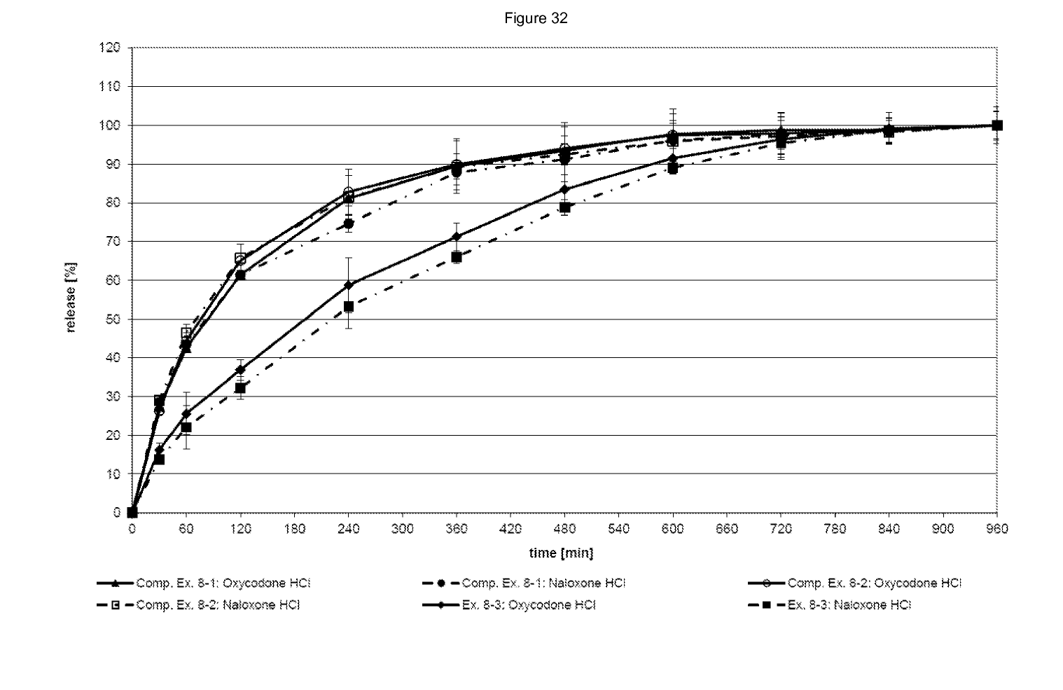

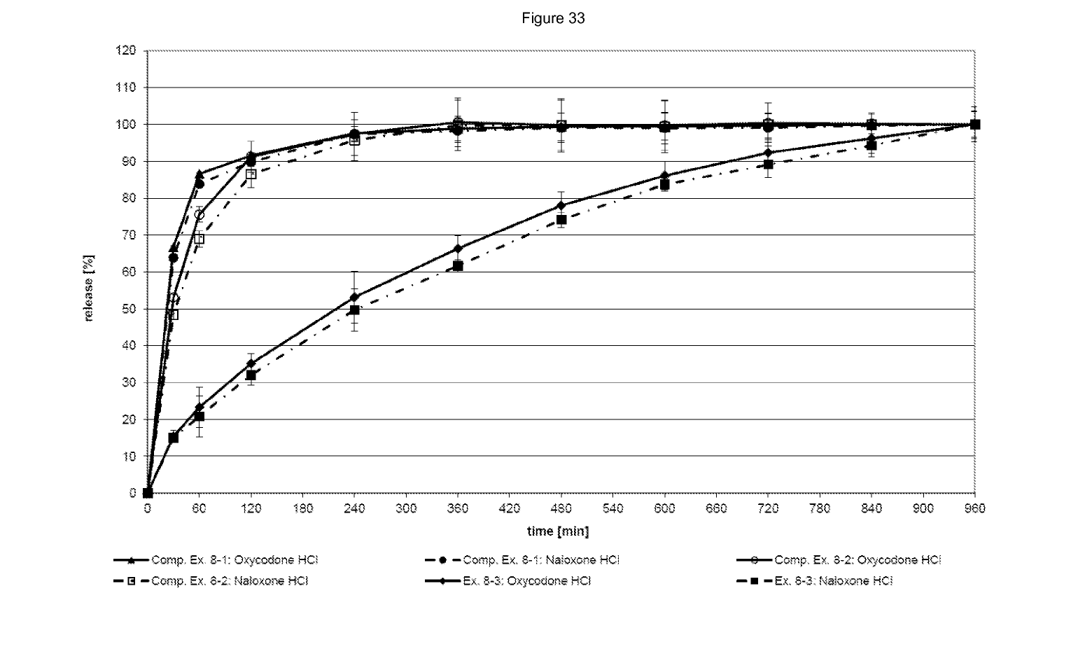

The in vitro release profiles (normalized values) of the pharmaceutical dosage forms according to Examples 8-1 to 8-3 are displayed in FIGS. 30 to 33:
FIG. 30: drug release of Oxycodone and Naloxone of intact tablets in simulated gastric fluids (HCl)
FIG. 31: drug release of Oxycodone and Naloxone of intact tablets in 40% ethanol
FIG. 32: drug release of Oxycodone and Naloxone of manipulated tablets in simulated gastric fluids
FIG. 33: drug release of Oxycodone and Naloxone of manipulated tablets in 40% ethanol

As can be seen, the manipulated tablets according to Comparative Examples 8-1 and 8-2, respectively, lost their controlled release properties.

The manipulation of the tablets according to Example 8-3 had neither an effect on the shape of the tablets nor on their release properties.

1. A pharmaceutical dosage form for oral administration having a breaking strength of at least 300 N and comprising an opioid agonist, an opioid antagonist, and a polyalkylene oxide having an average molecular weight of at least 200,000 g/mol, wherein in accordance with Ph. Eur. the in vitro release profile of the opioid agonist essentially corresponds to the in vitro release profile of the opioid antagonist, and wherein the opioid agonist and the opioid antagonist are intimately mixed with one another and homogeneously dispersed in the polyalkylene oxide

2. The pharmaceutical dosage form according to claim 1, wherein at every point in time the in vitro release profile of the opioid agonist does not deviate by more than 10% from the in vitro release profile of the opioid antagonist.

3. The pharmaceutical dosage form according to claim 1, wherein the opioid agonist and the opioid antagonist are homogeneously distributed over the pharmaceutical dosage form or, when the pharmaceutical dosage form comprises a film coating, over the coated core of the pharmaceutical dosage form.

4. The pharmaceutical dosage form according to claim 1, wherein the opioid agonist and the opioid antagonist are embedded in a prolonged release matrix comprising the polyalkylene oxide.

5. The pharmaceutical dosage form according to claim 4, wherein the prolonged release matrix comprises an additional matrix polymer.

6. The pharmaceutical dosage form according to claim 1, which is configured for administration once daily or twice daily.

7. The pharmaceutical dosage form according to claim 1, which is monolithic.

8. The pharmaceutical dosage form according to claim 1, wherein the content of the polyalkylene oxide is at least 30 wt.-%, based on the total weight of the pharmaceutical dosage form.

9. The pharmaceutical dosage form according to claim 1, which is thermoformed.

10. The pharmaceutical dosage form according to claim 9, which is hot-melt extruded.

11. The pharmaceutical dosage form according to claim 1, which is tamper-resistant.

12. The pharmaceutical dosage form according to claim 1, wherein the opioid agonist is oxycodone or a physiologically acceptable salt thereof.

13. The pharmaceutical dosage form according to claim 1, wherein the opioid antagonist is selected from the group consisting of naltrexone, naloxone, nalmefene, cyclazacine, levallorphan, pharmaceutically acceptable salts thereof and mixtures thereof.

14. The pharmaceutical dosage form according to claim 1, which contains a plasticizer.

15. The pharmaceutical dosage form according to claim 1, which contains an antioxidant.

16. A method of treating pain in a patient in need thereof, said method comprising administering to said patient a pharmaceutical dosage form according to claim 1.
